(19) United States
(12) Patent Application Publication
(10) Pub. No.: US 2008/0027304 A1
(43) Pub. Date: Jan. 31, 2008

(54) INTRAOCULAR PRESSURE ATTENUATION DEVICE

(76) Inventors: Geoffrey B. Pardo, Cambridge, MA (US); Kevin G. Connors, Wellesley, MA (US); Crystal M. Cunanan, Mission Viejo, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(21) Appl. No.: 11/737,054

(22) Filed: Apr. 18, 2007

Related U.S. Application Data
(60) Provisional application No. 60/792,587, filed on Apr. 18, 2006.
(60) Provisional application No. 60/809,278, filed on May 31, 2006.

(51) Int. Cl. A61F 9/007; A61M 27/00

(52) U.S. Cl. 600/399; 604/521; 604/8; 623/4.1

(57) ABSTRACT

Described herein are devices and methods that dampen transient intraocular pressure including pressure spikes experienced by the eye. The illustrative embodiments attenuate pressure waves and, thus, reduce wall stresses in a non-compliant eye such that the optic nerve is protected from damage in an ocular hypertensive or glaucoma patient, or during traumatic ocular procedures, and the refractive disorders of myopia, hyperopia, and/or presbyopia are moderated or reversed. In one embodiment, a compressible attenuation device insertable within the chambers of the eye preferably has an expanded volume within the range of from about 0.01 cc to 7 cc. The attenuation device may include a valve for permitting filling of the attenuation device through a delivery system. In another embodiment, the attenuation device comprises a flexible housing and a high vapor pressure media having a vapor pressure approximately equal to the intraocular pressure of the eye and a permeability of less than 1 ml/day at body temperature through an outer wall of the flexible housing.

This application claims priority to U.S. Provisional Patent Application Ser. No. 60/792,587, filed Apr. 18, 2006, and claims priority to U.S. Provisional Patent Application Ser. No. 60/809,278, filed May 31, 2006; the disclosures of the aforementioned applications are hereby incorporated in their entirety herein by reference.

### FIELD

The present invention relates generally to methods and apparatus for attenuating and/or baffling transient pressure waves in relatively incompressible materials in the eye, and in particular to the treatment of disorders of the eye caused by fluctuations of intraocular pressure (IOP); and more specifically, to methods and devices for the diagnosis and treatment of ophthalmic disorders such as glaucoma, ocular hypertension, retinal detachment, retinal tears, retinal ischemia, retinal vein occlusion, retinal artery occlusion, macular edema, diabetic retinopathy, neovascularization of the optic nerve, subretinal neovascularization, chronic posterior uveitis, macular degeneration, myopia, hyperopia and presbyopia.

### BACKGROUND

Pressure waves are known to propagate through incompressible fluids in various organs of the body. These pressure waves may be caused by a number of events including events within the body, such as a beating heart, breathing in the lungs, peristalsis actions in the GI tract, movement of the muscles of the body, or events such as coughing, laughing, external trauma to the body, and movement of the body relative to gravity. As the elasticity of the surrounding tissues and organs—sometimes referred to as compliance—decreases, the propagation of these pressure waves increases. These pressure waves have many undesirable effects ranging from discomfort, to stress on the organs and tissue, to fluid leakage such as urinary incontinence, to renal failure, stroke, heart attack, visual impairment, refractive error and blindness.

Pressure accumulators and wave diffusers are types of devices that can modulate pressure waves in various non-analogous settings. Accumulator technology is well known and used in hydraulic systems in aircraft, manufacturing equipment, and water supply and distribution since the 1940s. Common types of accumulators include bladder accumulators, piston accumulators, non-separator (air over fluid), and weight loaded type accumulators.

Wave diffusers also affect the transmission of pressure waves in incompressible systems in various settings. The function of such diffusers is to interrupt the progress of a pressure wave and distribute the energy of the wave in so many directions so as to destroy the integrity of a uniform wave front and its resultant effects. Wave diffusers may be used to protect a specified area from the impact of a wave front.

Ocular disorders are a widespread problem in the United States and throughout the world, affecting people of all ages. Visual impairment, including blindness, can be the result of many different disorders including relatively benign conditions such as myopia, hyperopia and presbyopia, in addition to more devastating conditions such as glaucoma, ocular hypertension, macular degeneration, retinal detachment and retinal tears. Many of these conditions can stem from a lack of compliance in the eye that stimulates high and fluctuating pressures which in turn can damage key, vision-producing structures within the eye.

Ever since the recognition by Bannister in the 16^{th} century that certain forms of blindness were associated with a firm eye, opthalmologists have been trying to measure and reduce IOP. IOP has been commonly used to evaluate the health of the human eye and has been linked to disorders such as glaucoma, retinal detachment, retinal tears, macular degeneration and refractive error. Reducing IOP has also been the intended therapy to treat many of these disorders.

Historically, the most common method of measuring IOP has been pressing on the cornea of the eye (the anterior chamber) to judge the rigidity or compliance of the chamber. This approach eventually evolved into an instrument known as the Schiotz tonometer which was a metal plunger that was used to press the anterior chamber for several seconds and computed a pressure reading. In more recent times, the ophthalmologist measures IOP either by placing a plastic prism on the cornea or sending a puff of air onto the cornea. These tests compute pressure by measuring the amount of force required to deform the cornea of the eye. When the pressure required to deform the cornea is applied a certain amount equilibrates to the pressure inside of the eye, an intraocular pressure measurement is recorded. In essence, the tests are measuring the rigidity or compliance of the eye to compute IOP. A compliant cornea would be indicative of low or normal pressure; a rigid eye would be indicative of high pressure “Normal” IOP is between 15-21 mmHg, but can vary greatly during different times of the day or as a result of varying corneal thickness. A measurement of more than 21 mmHg is not necessarily indicative of glaucoma; rather, it suggests that the patient has ocular hypertension.

It should be noted that there are many problems that have been reported in the measurement of intraocular pressure using tonometry. First, it is known that pressure is dynamic and varies throughout the course of the day and at night. Straining, blinking and eye rubbing can cause increases in eye pressure, and other activities like drinking water, tightening a necktie and blowing into a musical instrument can cause dramatic increases in IOP, none of which is captured during an office visit. Similarly, anatomical differences, such as corneal thickness, can distort pressure readings. It is becoming increasingly accepted that traditional forms of measurement are inadequate and may not precisely identify what the cause of the ocular disorder is.

A number of attempts have been made to reduce IOP and combat ocular disorders such as glaucoma, including the administration of drugs and surgical intervention. One such attempt involves the use of pharmaceutical drugs which typically act to limit the production of aqueous humor, or increase the outflow of aqueous humor via the different drainage ports in the eye. While drugs have been able to lower mean IOP in many patients, they have a number of drawbacks. First, compliance in taking the medicine is an issue, particularly when patients are on multiple medications as is often the case in glaucoma therapy. Second, these drugs can have systemic side effects, as in the case of beta blockers (cardiac failure), alpha agonists (allergies), prostaglandins (blurred vision, epithelial lesions, foreign body sensation).

Despite the primary role of medicines in the management of glaucoma, there are circumstances in which the physician must look to more aggressive means for controlling the disease.

Laser trabeculoplasty is a commonly used tool for the management of several types of open angle glaucoma. In this procedure, laser energy is applied directly to the trabecular meshwork through a series of 50-100 “burns.” Treatment seeks to re-establish flow of aqueous humor through the trabecular meshwork. The effectiveness of the procedure varies from patient to patient, although on average results in a 7 mmHg reduction in pressure for POAG patients. Some long-term studies have shown that pressure is controlled in only 45%-55% of treated patients at five years.

Some forms of angle-closure glaucoma can be addressed with a laser iridotomy procedure. This procedure utilizes an argon or Nd:YAG laser to create a hole in the iris to allow the flow of aqueous between the posterior and anterior chambers.

Laser cyclophotocoagulation reduces the amount of fluid produced in the eye through the destruction of the ciliary processes, as opposed to increasing fluid outflow. Using an 810 nm infrared diode laser in conjunction with a probe, the surgeon is able to precisely target and ablate the cells in the ciliary body that produce fluid.

In cases where medical or laser intervention is inadequate, surgical procedures may represent the last chance for the preservation of vision. For instance, trabeculectomy involves the creation of a new drain in the trabecular meshwork and the sclera. This procedure is the primary surgical method for the treatment of open-angle glaucoma and an estimated 125,000 are performed annually in the United States. Notably, the post-operative care is significant and generally requires office visits as often as once or twice per week for the first four to six weeks. While the introduction of antimetabolites has improved the success rate of the trabeculectomy, their use is also associated with a higher incidence of complications associated with over-filtration, such as hypotony (abnormally low IOP) and the long-term risk of serious ocular infection. In addition, longer term studies continue to suggest that as many as half of treated patients will eventually exhibit some loss of pressure control or further progression of the disease.

Seeking to avoid the complications associated with filtration surgeries such as the trabeculectomy, some surgeons have looked to “non-penetrating” techniques to manage later-stage glaucoma cases. The deep sclerectomy is a procedure in which a small flap is created in the sclera (the white part of the eye) followed by the “un-roofing” of the outer wall of Schlemm's canal and the exposure of Descemet's membrane, effectively creating a fluid drainage channel. Physicians have experimented with this procedure for many years, although a high failure rate was associated with the body's healing response that often resulted in closure of the pathway.

The viscocanalostomy is another non-penetrating procedure for the treatment of glaucoma. The procedure also attempts to reroute the aqueous flow through the creation of a window in Descemet's membrane, effectively bypassing the trabecular meshwork.

In some circumstances, a physician is unable to perform a trabeculectomy using the existing tissue in the eye. Several types of artificial drainage tubes/shunts, including the Molteno, Baerveldt, and Ahmed implants, have gained increased acceptance in the management of more complex glaucoma cases that may not respond well to a trabeculectomy or have already failed standard procedures. The artificial tube, usually composed of plastic, is generally implanted in the anterior chamber of the eye and drains to an external reservoir.

The intent of the treatment methods described to date either reduce the inflow of aqueous humor into the anterior chamber or increase the outflow of aqueous humor from the anterior chamber. The disadvantages and limitations of the prior art treatments are numerous and include:
require a high level of patient compliance
lack clinical efficacy
can be costly to the patient
have a high rate of side effects

These prior art approaches do not address the reduction in dynamic compliance which results in increased intraocular pressure.

### SUMMARY

Embodiments described herein are directed to methods and apparatus for measuring and/or attenuating and/or baffling transient pressure waves in the eye, and, in particular, to the treatment of disorders of the eye exacerbated by fluctuations in intraocular pressure. The embodiments described herein include devices and methods that dampen transient intraocular pressure including pressure spikes experienced by the eye. During a high frequency transient pressure event, the eye becomes a relatively non-compliant environment due to a number of factors including the ocular skeletal structure, the compressive loads of contracting tissues bounding the eye, the decreased compliance of the musculature, nerve or connective tissue of the eye or vascular hypertension. The factors contributing to the reduced compliance of the eye are aging, anatomic abnormalities or trauma to the structures of the eye.

The illustrative embodiments attenuate pressure waves and, thus, reduce wall stresses in a non-compliant eye such that optic nerve is protected from damage in an ocular hypertensive or glaucoma patient. The attenuation of pressure waves also prevents the blood vessels in the back of the eye from bursting or leaking, as happens in age-related macular degeneration patients, prevents the retina from tearing at the back of the eye, and reduces or eliminates the stretching of refractive structures of the eye including the sclera, the cornea, the crystalline lens, the ciliary body and the capsular bag. By attenuating the pressure waves and reducing or eliminating the stretch of these tissues, the progression of refractive disorders such as myopia, hyperopia and presbyopia is halted and possibly reversed.

In one embodiment, there is provided a compressible attenuation device insertable within the chambers of the eye preferably has an expanded volume within the range of from about 0.01 cc to 7 cc. The attenuation device may include a valve for permitting filling of the attenuation device through a delivery system.

In another embodiment, the attenuation device comprises a flexible housing and a high vapor pressure media having a vapor pressure approximately equal to the intraocular pressure of the eye and a permeability of less than 1 ml/day at body temperature through an outer wall of the flexible housing.

Further features and advantages of the present invention will become apparent to those of skill in the art in view of the detailed description of preferred embodiments which follows, when considered together with the attached drawings and claims.

### BRIEF DESCRIPTION OF THE DRAWINGS

FIG. 1A is a schematic cross-sectional view of the eye. The diagram shows the major structures within the eye, including the anterior chamber, the posterior chamber and the vitreous humor.

FIG. 1B is a schematic cross-sectional view of the eye which shows the flow of aqueous humor within the different chambers of the eye.

FIG. 2A illustrates intraocular pressures spikes that occur during continuous pressure monitoring of the eye. The spikes occur when an instrument is pressed on the anterior chamber of the eye at regular intervals.

FIG. 2B illustrates the dynamic intraocular pressure changes that occur over a 24 hour period.

FIG. 3A is a schematic view of one embodiment of an accumulator.

FIG. 3B is a schematic view of a simple accumulator.

FIG. 4 is a graph illustrating the effect on intraocular pressure of the presence of an implanted attenuation device.

FIG. 5A is a screenshot of the model eye test system when a 30 mmHg pressure pulse was applied to the chamber.

FIG. 5B is a screenshot of the test system with an air bubble as a simulated pressure attenuation device when a 30 mmHg pressure pulse was applied to the chamber.

FIG. 6a is a schematic top plan view of an inflatable attenuation device.

FIG. 6B is a side elevational cross-section through the attenuation device of FIG. 6A.

FIG. 7A is a schematic view of a toroidal shaped attenuation device.

FIG. 7B is a side elevational cross-section view through one embodiment of the attenuation device of FIG. 7A.

FIG. 7C is a side elevational cross-section view through one embodiment of the attenuation device of FIG. 7D.

FIG. 7D is a schematic view of a toroidal shaped attenuation device.

FIG. 8A is a schematic view of a toroidal shaped attenuation device as in FIG. 7A, with an integral baffle therein.

FIG. 8B is a side elevational cross-section view through one embodiment of the attenuation device of FIG. 8A.

FIG. 9 is a schematic illustration of the attenuation device disrupting the unitary progression of a pressure wave front.

FIG. 10A illustrates a cross-section of one embodiment of an attenuation device that can be implanted in the eye.

FIG. 10B illustrates a top-down view of one embodiment of an attenuation device that can be implanted in any chamber of the eye and that floats freely in the eye. The center portion can either correct for refractive error or be optically transparent.

FIGS. 10C thru 10H illustrate top-down views of different embodiments of attenuation devices that can be implanted in any chamber of the eye.

FIGS. 10I thru 10K illustrate cross-sectional views of different embodiments of attenuation devices that can be implanted in the anterior chamber of the eye.

FIGS. 11A thru C illustrate cross-sectional views of different embodiments of attenuation devices that can be implanted in the posterior chamber of the eye, anterior to the capsular bag and posterior to the iris.

FIGS. 12A and B illustrate cross-sectional views of different embodiments of attenuation devices that can be implanted in the capsular bag of the posterior chamber of the eye.

FIGS. 13A and B illustrate top-down views of different attenuation devices that can be implanted into the capsular bag of the posterior chamber.

FIG. 14A illustrates a cross-sectional view of one embodiment of an attenuation device that can be implanted in the vitreous humor and float freely in the vitreous humor.

FIGS. 14B thru E illustrate cross-sectional views of different embodiments of attenuation devices that can be implanted into the vitreous humor.

FIG. 15 illustrates cross-sectional view of an embodiment of an attenuation device that can be implanted into the vitreous humor and anchored in position with a cap.

FIGS. 16A-D are schematic representations of a variety of inflatable attenuation devices.

FIG. 17A is a side elevational schematic view of a bellows-type mechanically assisted attenuation device in an expanded configuration.

FIG. 17B is a side elevational schematic view of the attenuation device of FIG. 17A, in a compressed configuration attenuating a pressure spike.

FIG. 18 is a side elevational schematic view of a self-expanding graft type mechanically assisted attenuation device.

FIG. 19A is a side elevational schematic view of a multiple chamber attenuation device.

FIG. 19B is a schematic illustration of another multiple chamber attenuation device in a deployed orientation.

FIG. 20 illustrates a cross-sectional view of an aqueous shunt that contains a pressure attenuator as part of the drainage port.

FIG. 21A illustrates a cross-sectional view of an attenuator that has been implanted into the choroidal space.

FIG. 21B illustrates a cross-sectional view of an attenuator that has been implanted into the choroidal space and has a valve that extends into the sclera of the eye.

FIG. 21C is a schematic cross-sectional view illustrating a tubular attenuation device therein.

FIGS. 22A through 22D illustrate the positioning of an inflated attenuation device in the eye.

FIG. 23A is a side elevational schematic view of one embodiment of an attenuation device introducer.

FIG. 23B is a side elevational schematic view of an alternative embodiment of an attenuation device introducer.

FIG. 23C is a cross-section through the line 23C-23C in FIG. 23A.

FIG. 24A is a schematic representation of the delivery system of FIG. 23A trans-sclerally positioned within the eye.

FIG. 24B is a schematic illustration as in FIG. 24A, with the attenuation device inflated.

FIG. 25A is an elevated side view of one embodiment of a delivery system for the attenuation device.

FIG. 25B is an elevated side view of one embodiment of a delivery system for the attenuation device with the attenuation device exposed and ejected.

FIG. 26A is an elevated side view of one embodiment of a delivery system for the attenuation device.

FIG. 26B is an elevated side view of the inflatable attenuation device in FIG. 26A with the sheath slid proximally and the attenuation device exposed.

FIG. 27A is a fragmentary schematic view of the filling tube of a delivery system engaged within the valve of an attenuation device.

FIG. 27B is a fragmentary schematic view as in FIG. 27A, with the filling tube proximally retracted from the valve.

FIGS. 28A-8E schematically illustrate different valve constructions for an inflatable attenuation device.

FIG. 29A is a schematic top plan view of an inflatable attenuation device with a duckbill valve design.

FIG. 29B is a close-up view of the duckbill valve in FIG. 29A.

FIG. 30A is a schematic top plan view of an inflatable attenuation device with a ring valve design.

FIG. 30B is a schematic top plan view of an inflatable attenuation device with a fill/plug design.

FIG. 30C is a schematic top plan view of an inflatable attenuation device with a dome valve design.

FIG. 31A is a schematic top plan view of an inflatable attenuation device with a valve that prevents the influx and/or efflux of media to/from the attenuation device.

FIG. 31B is a cross-section through the line 31B-31B in FIG. 31A.

FIG. 32 is a schematic top plan view of a valve with two duckbill structures that prevent the flow of media in both directions.

FIG. 33 is a side elevational schematic view of an attenuation device removal system.

FIG. 34A is a side elevational schematic view of an inflatable balloon-type attenuation device, having a locatable balloon valve thereon.

FIG. 34B is a schematic perspective view of the attenuation device of FIG. 34A, aligned with the distal end of a delivery or removal system.

FIG. 35A is a fragmentary cross-sectional view through the distal end of a delivery or removal system, and the proximal end of the valve on an attenuation device, illustrating the valve in a filling or draining orientation.

FIG. 35B is a fragmentary cross-section as in FIG. 35A, showing the valve in a sealed orientation.

FIG. 36A is a schematic cross-sectional view through one embodiment of an implantable self-inflating attenuation device.

FIG. 36B is a schematic cross-sectional view through one embodiment of an implantable self-inflating attenuation device.

FIG. 36C is a schematic cross-sectional view through one embodiment of an implantable self-inflating attenuation device.

FIG. 37A is side elevational schematic view of a delivery system for deploying an implantable self-inflating attenuation device.

FIG. 37B is a cross-section through the line 37B-37B in FIG. 37A.

FIG. 37C is a schematic cross-sectional view of one embodiment of an implantable self-inflating attenuation device wrapped around the delivery system shown in FIG. 37D.

FIG. 37D is an elevated schematic view of a delivery system for deploying an implantable self-inflating attenuation device.

FIGS. 38A and 38B illustrate the connective and elastic tissues in the eye.

### DETAILED DESCRIPTION OF THE PREFERRED EMBODIMENT

Embodiments of the present invention are directed to methods and apparatus for measuring and/or attenuating and/or baffling transient pressure waves in relatively incompressible materials in organs of the body. Illustrative embodiments of the present invention discussed below relate generally to the fields of opthalmology, and in particular to the treatment of disorders of the eye exacerbated by fluctuations in intraocular pressure. However, as will be readily understood by those skilled in the art, and as described below, the present invention is not limited to the fields of opthalmology and methods and apparatus of embodiments of the present invention may be used in other organs of the body as well to attenuate and/or baffle pressure transients or reversibly occupy intraorgan space.

The embodiments described herein include devices and methods that dampen transient intraocular pressure including pressure spikes experienced by the eye. During a high frequency transient pressure event, the eye becomes a relatively non-compliant environment due to a number of factors including the ocular skeletal structure, the compressive loads of contracting tissues bounding the eye, the decreased compliance of the musculature, nerve or connective tissue of the eye or vascular hypertension. The factors contributing to the reduced compliance of the eye are aging, anatomic abnormalities or trauma to the structures of the eye. The illustrative embodiments attenuate pressure waves in a non-compliant eye such that the optic nerve is protected from damage in an ocular hypertensive or glaucoma patient; attenuate pressure waves such that the blood vessels in the back of the eye are prevented from bursting or leaking, as happens in age-related macular degeneration patients; attenuate pressure waves such that the retina is prevented from tearing at the back of the eye; and attenuate pressure waves that stretch refractive structures of the eye which include the sclera, the cornea, the crystalline lens, the ciliary body and the capsular bag. By attenuating the pressure waves and reducing or eliminating the stretch of these tissues, the progression of refractive disorders such as myopia, hyperopia and presbyopia is halted and possibly reversed.

There have been examples in the clinical literature demonstrating the effect that gas in the eye has on pressure. Tsilimbaris, et al showed in their study using Goldmann applanation tonometry (Current Eye Research 2002, Vol 24, No. 3, pp 202-205) that gas injected into the vitreous following vitrectomy surgery attenuated ocular wall pulsation. This attenuation effect correlated with the disappearance of the gas bubble—in other words, as the gas bubble disappeared the ocular pulse returned. While the bubble attenuated pulse it did not affect mean intra-ocular pressure. The article noted that increasing the elasticity of the eyeball could have important effects on macular degeneration as it is thought that reduced scleral elasticity can increase resistance to blood inflow—an important contributor to the pathogenesis of AMD. Other authors such as Lim (Archives of Opthalmology, 1990, Volume 108 (5), pp 684-688) and Poliner (Archives of Opthalmology, Volume 105, February 1987) have also noted the difficulty in ascertaining IOP in patients following gas injection in vitrectomy surgery. They note that the IOP is almost always underestimated by as much as 12 mmHg. This can be explained as the result of the compressibility of the gas bubble, and the absorption or attenuation of the force used to measure pressure. However, no one has shown that the attenuation effects of an air bubble could be used therapeutically to dampen pressure waves and treat ocular disorders.

The use of a pressure attenuation device to attenuate dynamic pressure pulses in the eye was studied using computational modeling. Literature values for ocular tissue properties were used in a model eye of 5.5 mL volume. The effects of changing pressures in the eye were examined using a pressure loading method, where additional pressure was put into the eye of a fixed size. The effects of pressure attenuation were studied by affixing chambers of various compliances to this model to determine the overall influence of the device on the mean intraocular pressure and the resulting wall stresses which occur at the position of the optic nerve.

This study found that the wall stresses which result from these pressure pulses are extremely high, where a 30 mmHg baseline condition with 10 mmHg pressure spikes results in a wall stress at the optic nerve head of over 457 mmHg. A compliant device capable of attenuating 0.40 mL/kPa was able to reduce the peak wall stress by over 72 mmHg, or 16%. In an eye with a baseline pressure of 20 mmHg and with 10 mmHg pressure spikes, the wall stress at the optic nerve head is over 326 mmHg and a compliant device was able to reduce the peak wall stresses by over 62 mmHg, or 19%. The stresses at the optic nerve head are large because of the large ratio of eye radius to eye wall thickness, which is about 20:1. Therefore, with small deviations in size, such as by filling the eye with small volumes of fluid, the translated effect on the wall stress is high.

Thus, considering the problem of pressure and pressure spikes within the eye, evaluating the resulting pressure-induced forces at the point of the optic nerve head is essential to understand the magnitude of the problem. Small changes in measured intraocular pressures translate into large changes in wall stresses at the point of the optic nerve head, the site of the degeneration of the retinal ganglion cells. Therefore, designing attenuation devices which can reduce wall stresses to specified levels is an important design consideration. Devices which reduce wall stresses from peak values of over 450 mmHg and can reduce wall stress by 50 mmHg, 75 mmHg, 100 mmHg, or even 150 mmHg or more are desirable.

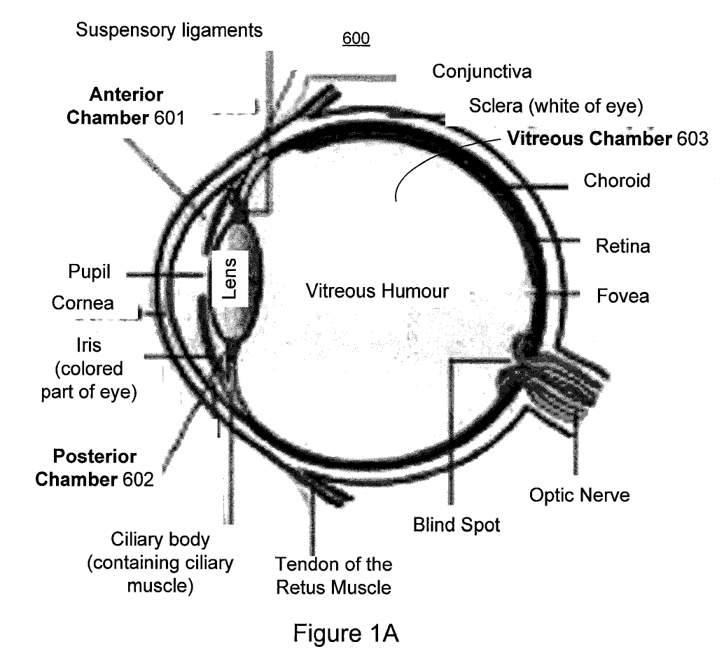

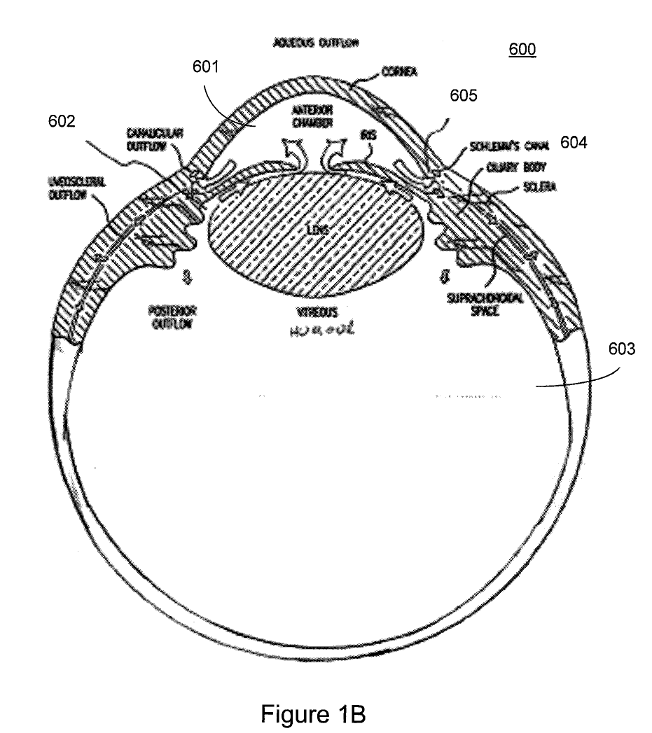

The eye 600 is comprised of three chambers of fluid as depicted in FIGS. 1A and 1B. There is the Anterior Chamber 601 between the cornea and iris, the Posterior Chamber 602 between the iris, zonule fibers and lens, and the Vitreous Chamber 603 between the crystalline lens and the retina. The Anterior and Posterior chambers are filled with aqueous humor, whereas the vitreous chamber is filled with a more viscous fluid, the vitreous humor. The aqueous humor and vitreous humor are virtually incompressible in the typical pressure ranges present within the human eye.

Compliance of the eye is defined as the ratio of the change in volume to the change in pressure, and the static compliance of the eye is measured during a typical tonometric evaluation. The static compliance index is measured by placing a mechanical force on the cornea and allowing the pressures to equilibrate for a time period of approximately 3-5 seconds. The static compliance index is calculated using standard applanation tonometry. Normal, compliant eyes will typically exhibit resting pressures from 10 mmHg to 21 mmHg during an office visit. Abnormal, rigid eyes will typically have pressures above 21 mmHg. The steady state compliance of the eye is used to diagnose patients with problems such as damage to the optic nerves, macular and retinal problems, refractive problems, and damage to other critical structures of the eye.

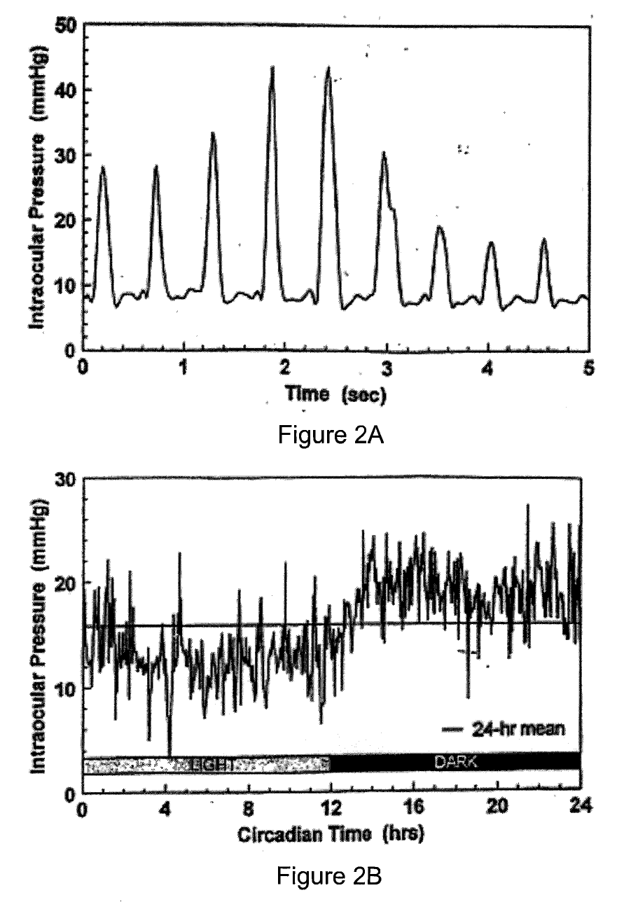

In general, intraocular pressure spikes result from volumetric tissue displacement in response to gravity, muscular activity, vascular pulsation, rapid acceleration, blinking, straining, eye rubbing and other activities. The lack of compliance of the eye and the fluid contained in the eye with respect to events of high frequency, result in minimal fluidic pressure attenuation of the higher frequency pressure wave(s) and results in high intraocular pressures that are directly transmitted to the structures of the eye. Under steady state conditions, as shown in FIG. 1B, fluid passes (as indicated by the arrows) from the posterior chamber 602, to the anterior chamber 601, through the trabecular meshwork 605 and out Schlemm's canal 604, in effect, a volumetric pressure relief mechanism allowing a proportional volume of fluid to escape the eye, to lower the intraocular pressure to a tolerable level. However, this fluid flow relief mechanism is not “fast” enough to work for rapid pressure change as shown in FIG. 2A where a continuous pressure monitor in the eye was used to monitor pressure spikes during periods when external pressure was applied to the eye. As demonstrated by the pressure spikes which can represent increases of more than 30 mmHg, the fluid flow in the eye does not adjust quickly enough to prevent these pressure changes. These pressure fluctuations occur throughout the course of the day as demonstrated in FIG. 2B which uses a continuous pressure monitor to show the swings in pressure over the course of a 24 hour period. It should also be noted that these graphs show pressure in a normal, compliant eye. It is reasonable to expect that in a non-compliant eye, such as a glaucomatous eye, the intensity of the pressure spikes will be greater.

It is recognized herein that for the vast majority of patients suffering from problems of optical disorders, the cause and/or contributor to the dysfunction is a reduction of overall dynamic eye compliance rather than or in addition to steady state eye compliance. These patients may often have eyes that are compliant in steady state conditions (for example, normal tension glaucoma), but have become non dynamically compliant when subjected to external pressure events having a short duration of, for example, less than 10 seconds or in some cases less than 5 seconds, less than 2 seconds, less than 0.5 seconds, or less than 0.01 seconds. Reduction in dynamic compliance of the eye is often caused by some of the same conditions as reduction of steady state compliance including aging, use, distention, hypertension and trauma. The anatomical structure of the eye in relation to the eye socket, vascular structure and surrounding tissues causes external pressure to be exerted on the eye during talking, walking, laughing, sitting, moving, turning, swallowing, sleeping, straining and blinking, as well as during traumatic ocular procedures.

Certain embodiments described herein provide for methods and devices for measuring and reporting the dynamic compliance of the eye. One method of determining dynamic compliance includes implanting a pressure transducer into the eye which continuously monitors changes in pressure, which as described below could be included with a pressure attenuator device. The transducer device could take up to 2000 readings per second and wirelessly transmit that information to an external source. Alternatively, an external device is contemplated with pressure measurements up to 2000 readings per second during extended pressure displacement.

Additional embodiments provide methods and devices for treating and/or compensating for reduced dynamic compliance of the eye. In one embodiment, a device having a compressible element is placed within the human eye, in a manner that allows the compressible element to act as a pressure accumulator or attenuator to attenuate transient pressure events. The term accumulator refers generally to devices that attenuate pressure, force, or energy in a given locale by absorbing and/or shifting away said pressure, force, or energy from said locale. The term attenuator refers generally to devices that attenuate pressure, force, or energy by dissipating or dampening said pressure, force, or energy. Gases such as atmospheric air, carbon dioxide and nitrogen are very compressible in the pressure ranges typically encountered in the human eye, and these gases may be used in attenuation devices inserted in the eye. Furthermore, when compared to the tissues encompassing fluid, these gases are significantly more compliant than the immediate environment. The addition of a proportionately smaller volume of unpressurized gas acts as a low rate spring in series with the native fluidic circuit of the eye. Additional information on the basic scientific principles underlying pressure accumulators and methods for controlling transient changes in pressure can be found in E. BENJAMIN WYLIE ET AL., FLUID TRANSIENTS IN SYSTEMS § § 6, 10, 11, 13 (1993); the entirety of these sections are hereby incorporated by reference herein and made a part of this specification.

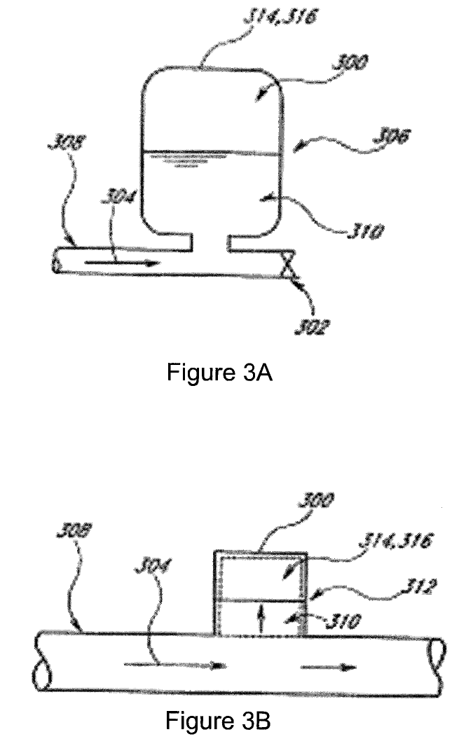

Accumulators can be designed to keep the pressure from exceeding a predetermined value or to prevent low pressures. Accumulators can be designed to protect against rapid transients as well as against longer-period surges in a system. One example of an accumulator is a closed container partially filled with the system liquid and topped with air or gas. The gas may be in contact with the liquid, in which case an air compressor, or gas supply, is used to maintain the proper mass of air or gas, or the gas may be separated from the liquid by a flexible membrane or a piston. The accumulator generally operates at the local system pressure. With reference to the embodiment illustrated in FIG. 3A, if the valve 302 of the accumulator 300 is closed abruptly the flow 304 enters the air chamber 306, the air is compressed, and the flow to the main pipeline 308 is gradually reduced as the pressure builds up, thereby provides a way to reduce the peak pressure in the chamber 306, the main pipeline 308, and other downstream plumbing and equipment.

With reference to the embodiment illustrated in FIG. 3B, a single accumulator 300 is assumed to have the same pressure throughout its volume at any given instant. Here, the compressibility of the liquid 310 in the vessel 312 is considered negligible compared with air compressibility. Assuming inertia and friction are negligible, the gas 314 is assumed to follow the reversible polytropic relation H_{A}Vⁿ=C_{A}, where H_{A} is the absolute head equal to the gauge plus barometric pressure heads, where Vⁿ is the gas volume 316, where n is the polytropic exponent, and where C_{A} is a constant. The exponent n depends on the thermodynamic process followed by the gas 314 in the vessel 312. If a perfect gas is assumed, at one extreme the process may be isothermal, n=1, or at the other limit it may be isentropic, in which case n=1.4 for air. It should be noted that computation of the aforementioned values, as well as analogous or related values, can be determined by those skilled in the art by taking into consideration the foregoing discussion.

In another embodiment, the compression of the enclosed volume of air creates heat that is dissipated into the relatively infinite heat sink of the body. The balance of the energy absorbed by the compressed air is simply returned at a different, lower frequency into the fluidic circuit when the gas is allowed to expand, as the surrounding tissues return to their initial positions. The addition of adequate local compliance can effectively attenuate transient intraocular pressure spikes to levels below where damage to the critical structures of the eye occurs.

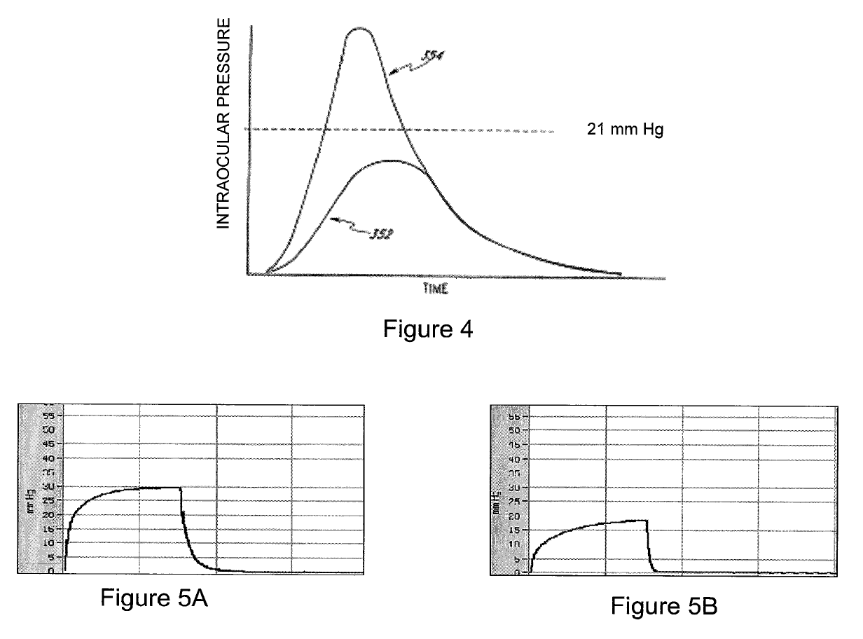

FIG. 4 illustrates the effect of an attenuation device on the intraocular pressure. Here, the intraocular pressure 352 with the attenuation device exhibits delayed rise and decay times and remains below the pressure of 21 mm Hg found in non-compliant eyes. This is in contrast to the intraocular pressure 354 which exceeds abnormally high pressure without an attenuation device.

FIGS. 5A-B illustrate pressure attenuation (i.e. pressure reduction) with an attenuation device. The data for these graphs were generated using a bench top eye simulation program. Here, the maximum spike pressure is 30 mmHg. The spike event duration is approximately 150 milliseconds, which is approximately equivalent to the duration of an ocular surface touch during a pressure measurement. With reference to FIG. 5A, a test was conducted with a 5.5 mL rigid plastic container filled with saline. A regulated pressure of 30 mmHg was introduced into the container via a controlled solenoid valve. A pressure transducer detected the pressure rise. Here, the pressure rise time (Tr) of the container pressure 422 to reach 30 mmHg was approximately 80 milliseconds. With reference to FIG. 5B, a similar test was conducted on a 5.5 mL rigid plastic container. Here, an air bubble simulating an attenuation device was placed inside the container filled with saline. During the spike event, the pressure inside the container reached 18 mmHg versus 30 mmHg for the unattenuated sample, resulting in a 40% reduction of pressure vs. baseline.

In another embodiment, an attenuation device is placed within the human eye. The attenuation device can be tethered or untethered in the eye and is intended to remain in the eye for a period of the duration of the intra-operative procedure up to a permanent implant, between several hours and several years, between one week and one year, or between one and six months. The attenuation device is preferably a small elastomeric air cell with a relaxed (unstretched) volume of between 0.001 and 7 cc, more preferably between 0.1 and 5 cc and more preferably, between 0.1 and 3 cc. The attenuation device is a unitary component but can be comprised of two or more subcomponents. The attenuation device has a substantially uniform wall thickness of between 0.0001 inch to 0.25 inch, more preferably between 0.0005 inch and 0.005 inch, but could be designed to vary greatly, and still perform the intended function. In the embodiment described above, attenuation devices having air cells that are free-floating in the eye have been described. In other embodiments, air cells or similar attenuation devices could be surgically affixed to the eye wall through the use of suture, staples, rivets, pincers, nails, screws and other accepted methods or attached to the iris, cornea, sclera, trabecular meshwork, posterior lens capsule or other anatomical structures within the eye. Other embodiments could also include attenuation devices with programmable, variable and adjustable buoyancy by using ballasting, specific inflation/deflation solutions, alternative materials of construction or by other means.

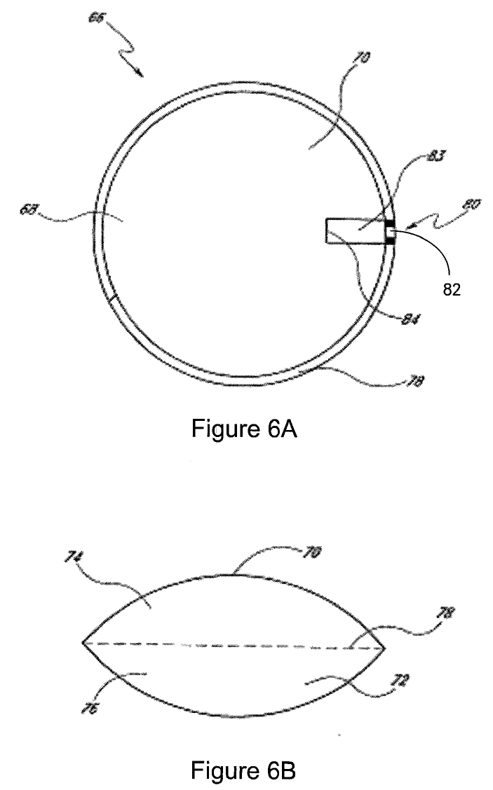

Referring to FIGS. 6A and 6B, there is illustrated one embodiment of an attenuation device 66 which comprises a moveable wall such as on an inflatable container 68. The inflatable container 68 is illustrated as having a generally circular profile, although other profiles may be utilized. The diameter of the inflatable container 68 may be varied within the range of from about 0.5 mm to about 25 mm, in an application involving the implantation of only a single attenuation device. Many embodiments of the inflatable containers 68 will have a diameter within the range from about 0.5 mm to about 25 mm, with a total volume within the ranges recited above. In general, the specific dimensions and configuration of the inflatable container 68 are selected to produce an attenuation device having a desired volume and a desired dynamic compression range, and may be varied from spherical to relatively flat as will be apparent to those of skill in the art based upon the disclosure herein. In certain embodiments, two or three or more discreet inflatable containers 68 are utilized. The sum of the volumes of the multiple containers will equal the desired uncompressed displacement.

The inflatable container 68 illustrated in FIGS. 6A and 6B comprise a flexible wall 70, for separating the compressible contents of the attenuation device 66 from the external environment. Flexible wall 70 comprises a first component 74 and second component 76 bonded together such as by a seam 78. In the illustrated embodiment, the first component 74 and second component 76 are essentially identical, such that the seam 78 is formed on the outer periphery of the inflatable container 68. Seam 78 may be accomplished in any of a variety of manners known in the medical device bonding arts, such as heat bonding, adhesive bonding, solvent bonding, RF or laser welding, or others known in the art.

The flexible wall 70 formed by a bonded first component 74 and second component 76 define an interior cavity 72. As is discussed elsewhere herein, interior cavity 72 preferably comprises a compressible media, such as gas, or foam. Other media or structures capable of reduction in volume through a mechanism other than strict compression may also be used. For example, a material capable of undergoing a phase change from a first, higher volume phase to a second, lower volume phase under the temperature and pressure ranges experienced in the eye-may also be used.

In order to minimize trauma during delivery of the attenuation device 66, the attenuation device is preferably expandable from a first, reduced cross-sectional configuration to a second, enlarged cross-sectional configuration. The attenuation device 66 may thus be trans-sclerally deployed into the eye in its first configuration, and enlarged to its second configuration once positioned within the eye to accomplish the pressure attenuation function. Preferably, a crossing profile or a greatest cross-sectional configuration of the attenuation device 66 when in the first configuration is no greater than about 10 mm, and, preferably, no greater than about 3 mm. This may be accomplished, for example, by rolling a deflated inflatable container 68 about a longitudinal axis, while the interior cavity 72 is evacuated.

Once positioned within the eye, the interior cavity 72 is filled with the compressible media to produce a functional attenuation device 66. Fill pressures are contemplated to between 0.1 and 50 mmHg, and more preferably between 1 and 40 mmHg and fill volumes are contemplated to be between 0.01 cc and 7 cc, and more preferably between 0.1 cc and 3 cc. In general, the fill pressure and volume are preferably no more than necessary to keep the attenuation device 66 inflated or partially inflated in the absence of pressure spikes. Excessive pressure and volume within the attenuation device 66 may shorten the dynamic range of the attenuation device 66, thereby lessening the sensitivity to attenuate pressure spikes. Pressures of less than 50 mmHg or even vacuums may be utilized if the structure of the attenuation device is sufficient to balance the negative pressure to produce a net force such that attenuation can occur. This may be accomplished, for example, in an embodiment where the attenuation device 66 is provided with a self-expandable support structure (e.g. nitinol wire frame), which provides a radially outwardly directed bias.

The resiliency of the material of the attenuation device, and the pressure and volume of the inflation media are preferably matched to produce a compression cycle time which is fast enough to allow the attenuation device to respond to increases in pressure while not having a clinically detrimental effect on normal fluid outflow through Schlemm's canal or other drainage outlets in the eye.

In one embodiment, the attenuation device comprises a flexible housing comprising an outer wall defining a chamber therein. The housing is configured to be introduced into an eye while in a first, introduction configuration and then at least partially inflated into a second, implanted configuration. The housing is at least partially inflated by injecting at least one high vapor pressure media having a vapor pressure approximately equal to the intraocular pressure of the eye and a permeability of less than about 1 ml/day at body temperature through the outer wall of the housing. The media causes a volume of a first gas to be driven through the housing until the partial pressure of the first gas inside the housing matches the partial pressure of the first gas within the eye.

The high vapor pressure media could be one or more of the following representative compounds, including heptafluoropropane, perfluorooctylbromide, perfluorohexane, perfluorodecalin, tetrafluoroethane, sulfur hexafluoride, hexafluoroethane, perfluoropropane, perfluorobutane, perfluoropentane, perfluoroheptane, perfluorooctane, octafluoropropane, decafluoro-n-butane, perfluoroperhydrophenanthrene, or other similar compounds.

To facilitate filling the interior cavity 72 following placement of the attenuation device 66 within the eye, the inflatable container 68 is preferably provided with a valve 80. In the illustrated embodiment, valve 80 is positioned across the seam 78, and may be held in place by the same bonding techniques utilized to form the seam 78. Valve 80 may be omitted in an embodiment in which the attenuation device 66 is self-expandable.

Valve 80 generally comprises an aperture 82, for receiving a filling tube there through. Aperture 82 is in fluid communication with the interior cavity 72 by way of a flow path 83. At least one closure member 84 is provided for permitting one way flow through flow path 83. In this manner, a delivery system and filling device can be utilized to displace closure member 84 and introduce compressible media into the interior cavity 72. Upon removal of the filling device, the closure member 84 prevents or inhibits the escape of compressible media from the interior cavity 72 through the flow path 83.

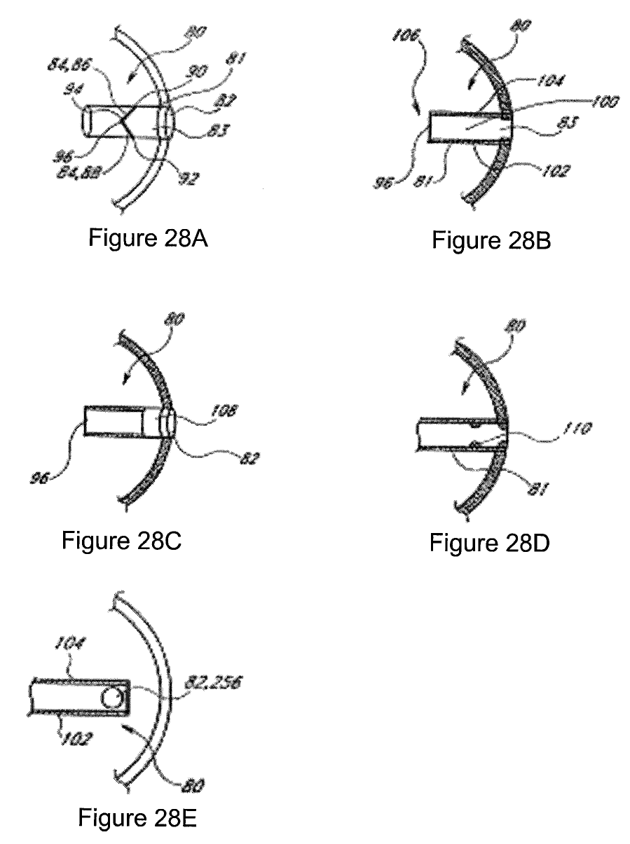

Thus, the closure member 84 is preferably movable between a first orientation in which it obstructs effluent flow through the flow path 83 and a second position in which it permits influent flow through the flow path 83. Preferably, the closure member 84 is biased in the first direction. Thus, forward flow may be accomplished by either mechanically moving the closure member 84 into the second position such as using a filling tube, or by moving the closure member 84 into the second position by exerting a sufficient pressure on the compressible media in flow path 83 to overcome the closure bias. Certain specific valve structures will be described in connection with FIGS. 28A-E, 29A-B and 30A-C below. However, any of a wide variety of valve designs may be utilized in the attenuation device 66 as will be apparent to those of ordinary skill in the art in view of the disclosure herein.

In one embodiment, the attenuation device comprises an air cell having of 0.0018 inch thick polymer sheets that have been bonded together to form a 1 cm circle in top view. The attenuation device can be made from polyurethane and is intended to be inflated to a volume less than 5 ml or generally within the range of about 0.3 to 2.5 ml. Integral to the sealing edge 78 of the attenuation device holds a port/valve 80 utilized in the placement, inflation and release of the attenuation device. Into the port/valve structure 80 is placed the distal end of a rigid fill tube (2 mm OD) 50. The valve 80 employed may be one of the valves described in U.S. Pat. No. 5,144,708, which is incorporated herein by reference. In another embodiment, the attenuation device may be ultrasonically, radio frequency, adhesively or heat sealed in situ following inflation, in which case the valve may be omitted.

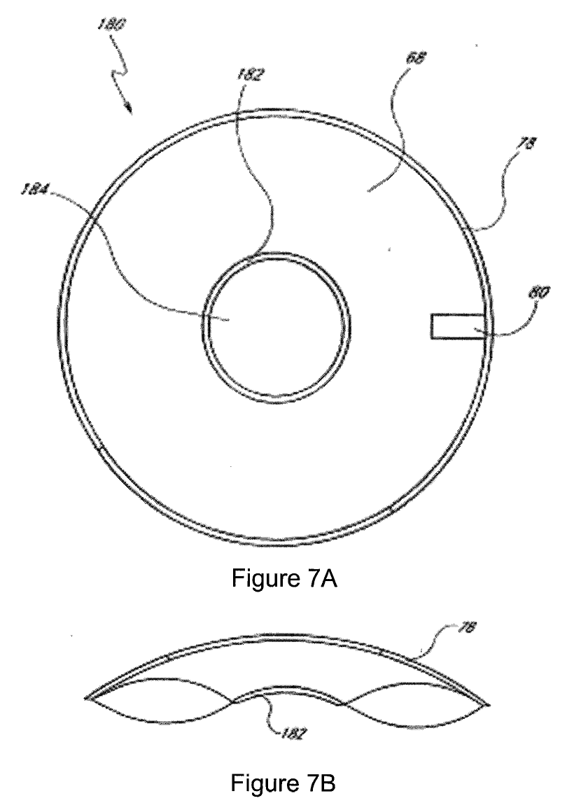

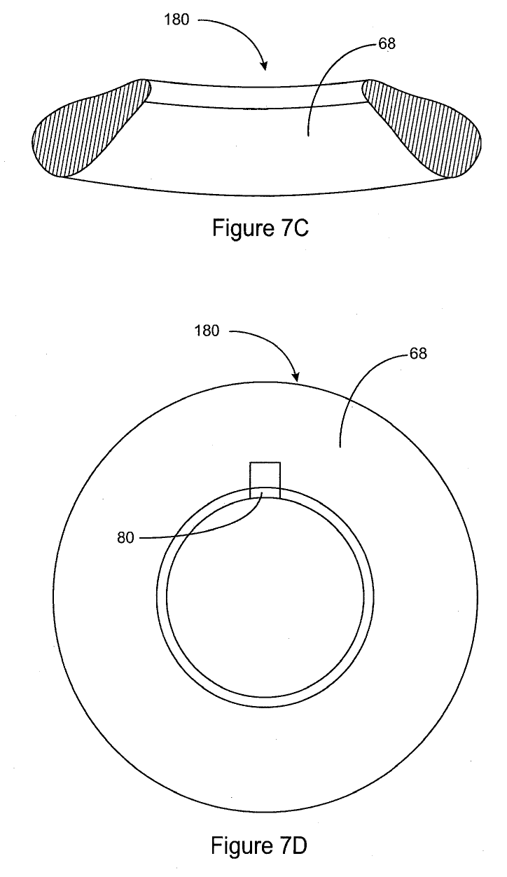

Referring to FIGS. 7A and 7B, there is illustrated a top plan view of one embodiment of an attenuation device 180. The attenuation device 180 comprises an inflatable body 68 generally as has been described. An outer seam 78 may be provided with a valve 80. In this embodiment, an inner seam 182 defines a central region 184. The outer seam 78 and inner seam 182 define a generally toroidal-shaped inflatable container 68. The central region 184 may comprise either a membrane or a central opening, depending upon the desired performance characteristics. The center hole may assist in the placement and location of the attenuation device within the eye, permit additional baffling of the pressure waves within the eye, minimize the attachment to structures within the eye by surface tension between the attenuation device and the wall of the eye, and allow for aqueous humor flow through the hole in the event that the attenuation device is in or near the angle of the anterior chamber or near the various drainage ports of the eye, such as the trabecular meshwork, Schlemm's canal, and the uveoscleral channels.

The central region 184 in FIG. 7A may also contain a lens that corrects for refractive error, including myopia, hyperopia or presbyopia. The lens may be made of a rigid, non-compliant material or a material that is able to flex to provide accommodation. The central region may also contain a lens that is ocularly neutral or has the same index of refraction as the vitreous and/or aqueous humour thereby not altering the pathway of light to the retina.

An alternative shape to the attenuation device 180 is provided in FIGS. 7C and 7D. The attenuation device 180 comprises an inflatable body 68 that is provided with a valve 80 along its inner diameter.

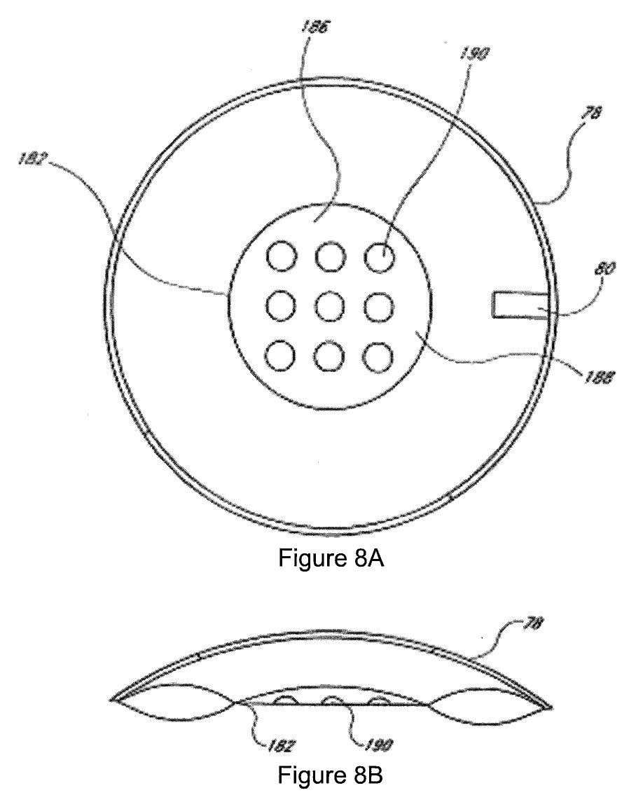

In one embodiment, illustrated in FIGS. 8A and 8B, the central region 184 comprises a baffle 186. The baffle 186 comprises a membrane 188 having a plurality of apertures 190 therein. In the illustrated embodiment, approximately nine round apertures 190 are provided, each having a diameter of about 0.04 inches. Generally at least about 9 apertures 190 are provided, and many embodiments include anywhere from about 1 to about 1000 apertures. The optimal number of apertures 190 and sum of the area of the apertures 190 compared to the total area of the baffle 186 may be optimized depending upon the desired performance characteristics. Apertures may have any of a variety of configurations, such as round holes, irregular openings, slits or others.

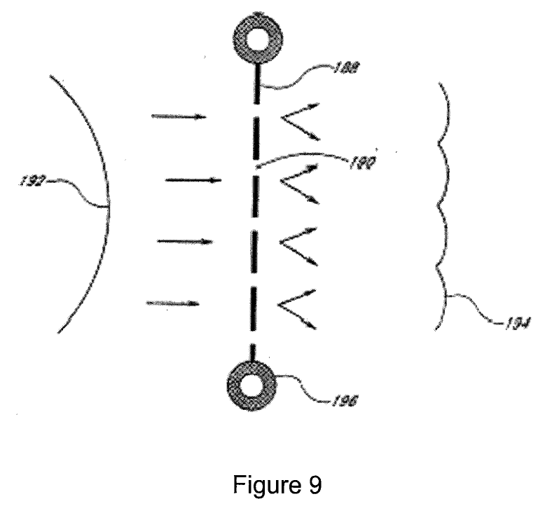

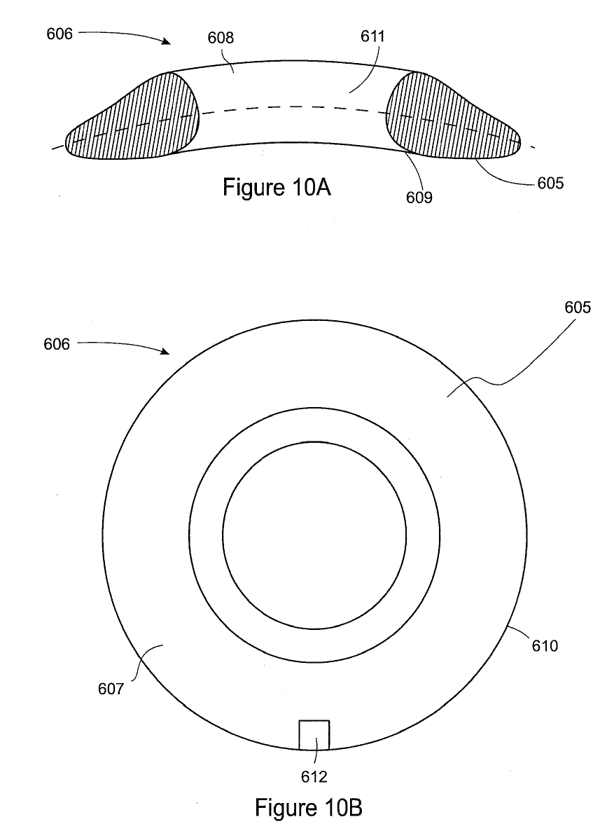

The wave diffuser function of the baffle 186 is schematically illustrated in FIG. 9. A wave front 192 may be generated by any of a wide variety of events, such as blinking, eye rubbing, coughing, sneezing, laughing, physical movement, muscle spasms or others as is understood. Since aqueous humor comprises essentially non-compressible fluid, and due to the low dynamic compliance of the eye the wave front 192 will propagate rapidly through the eye to impact structures such as the optic nerve, the retina, the cornea or blood vessels with the eye. Apparent transient pressure spikes as high as 30 mmHg or greater can be experienced during normal activities.

If the attenuation device 180, having a baffle 186 is positioned within the eye, the baffle 186 functions to disrupt the unitary progression of the wave front 192. The prediffusion wave front 192 is thus interrupted into a plurality of post-diffusion wave fronts 194 by the baffle 186. Although the sum of the resulting post-diffusion wave fronts 194 is essentially equal to the prediffusion wave front 192, the greater dispersion of force accomplished by the baffle 186 is believed to reduce the apparent magnitude of the wave front 192 as experienced by structural tissue within the eye.

As will be apparent in view of the foregoing, the baffle 186 may be constructed in any of a variety of manners and still accomplish the intended result. Thus, although the attenuation device 180 illustrated in FIGS. 7 and 8 comprise a generally toroidal-shaped inflatable container, any of a variety of other support structures may be utilized to maintain the baffle 186 in a useable configuration. The support 196 can comprise an inflatable tube, a resilient material such as nitinol wire, or other support structure as may be desired.

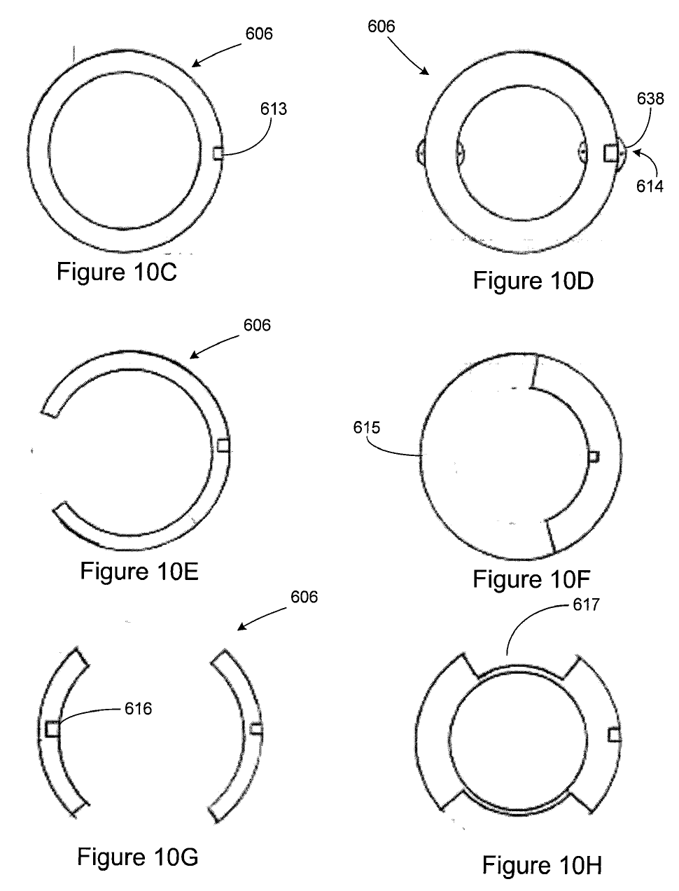

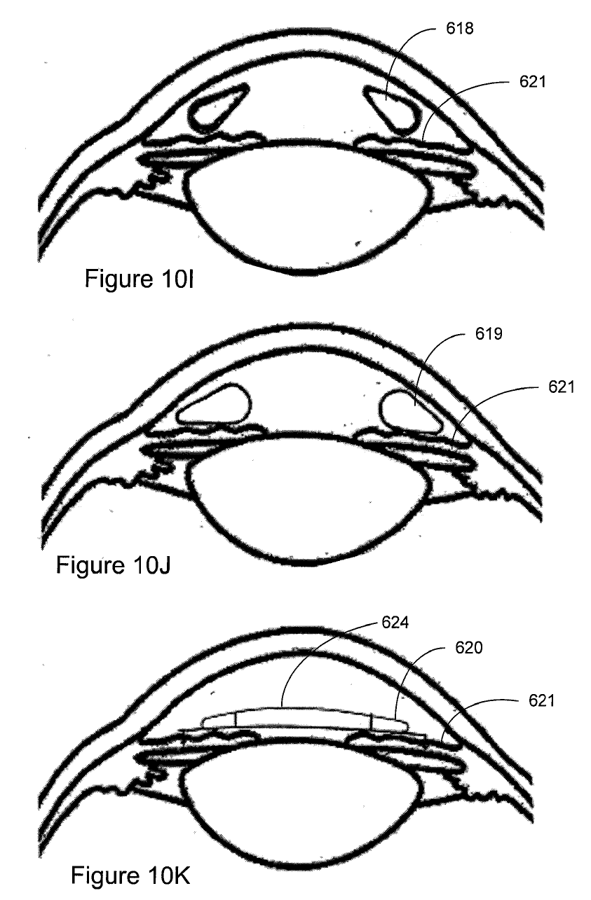

In another embodiment, the attenuation device comprises of an air cell in the shape of a donut, where the donut is inflated as shown in diagram 10A and B. FIG. 10B shows a fill valve port 612 on the outer edge of the device. FIG. 10D consists of an attenuation device that has anchors 614 located on either side of the device. FIG. 10E demonstrates a device that has a circumference of less than 360 degrees. FIG. 10F shows a device where the circumference of the air-filled portion may be less than 360 degrees and connected to a positioning ring 615 that extends the circumference to 360 degrees. FIG. 10G shows a double sided attenuator device with no connections between the air chambers and filling valves on each side 616. There may also be a single sided air chamber. FIG. 10H would be a double sided air chamber with members 617 that connect, fluidically or otherwise, the air chambers. These embodiments may be in any shape and are not limited to circular shapes.

FIGS. 10I through 10K shows a cross-section of the attenuation device when positioned in the anterior chamber of the eye. The placement of such a device takes advantage of standard delivery techniques used in the placement of phakic IOLs. The devices will typically be inserted through incisions less than 6 mm, more preferably between 0.5 and 3 mm. FIG. 10I shows a free-floating air cell 618 in which no portion of the device enters into the optical path. It is angled in such a way as to minimize contact with the iris 621. FIG. 10J shows an attenuator device 619 in which no part of the device enters into the optical path and which lays flat on the iris 621. Another embodiment of both 10I and 10J could include a lens which contains no power or which enhances vision. FIG. 10K shows an attenuator device that is anchored to the iris 621 in ways known to the art, for instance with pincers, staples, rivets, sutures, clips, nails, screws and other means of attachment (Willis, U.S. Pat. No. 7,008,449 or Worst U.S. Pat. No. 5,192,319). The air cell 620 in such an embodiment preferably surrounds the lens 624 which could have no optical power or correct for refractive error.

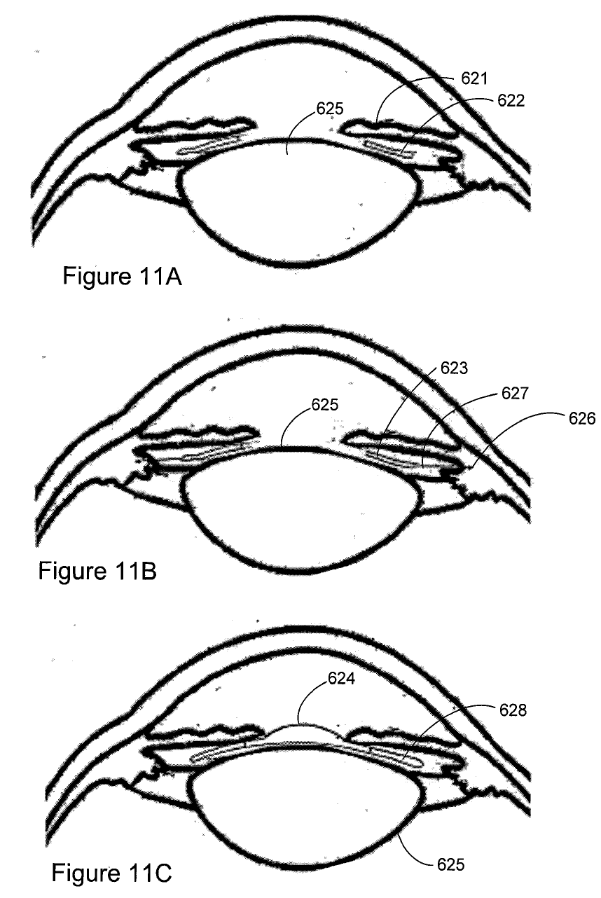

FIGS. 11A through 11C shows embodiments where the attenuation devices are located in the posterior chamber of the eye, posterior to the iris 621 and anterior to the capsular bag 625. FIG. 11A shows a free-floating attenuation device in which no part of the device 622 enters into the optical path. FIG. 11B shows a free-floating attenuation device 623 in which no part of the device enters into the optical path and in which there are anchors 627 into the ciliary bodies 626. The anchors comprise sutures, staples, or haptics, which press outward into the ciliary bodies, or other forms of anchoring known to those skilled in the art. FIG. 11C shows a free-floating attenuation device 628 in which a lens 624 covers the optical path. Such a lens may have no optical power or correct for refractive error.

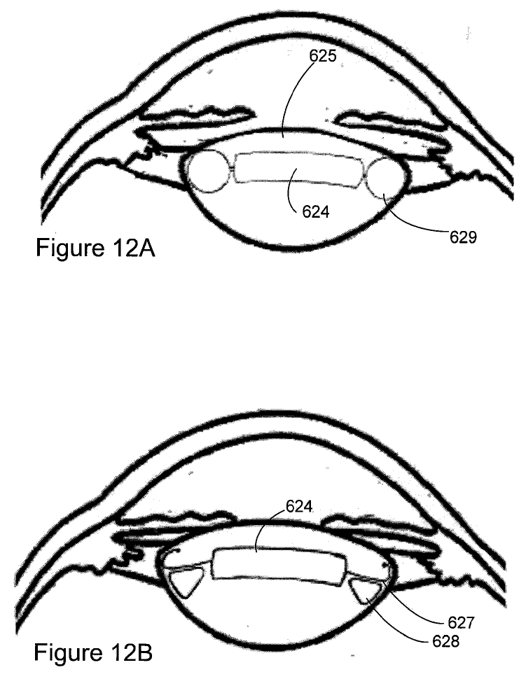

FIGS. 12A and B show embodiments where the attenuation device 629 is placed into the capsular bag 625 in the posterior chamber following a standard phacoemulsification technique and an insertion technique similar to that of intraocular lens placement for cataract surgery. FIG. 12A shows a cross-section of the device in which the haptics of the lens 624 are the air cells 629 of the attenuation device extending outward to the wall of the capsular bag. The lens portion 624 of the device may provide accommodation, and allow for the appropriate correction of refractive error. FIG. 12B shows a device that is placed into the capsular bag 625 in the posterior chamber and consists of a standard IOL 624 with haptics 627, and posterior to that an attenuation device 628 that can be connected or unconnected to the IOL.

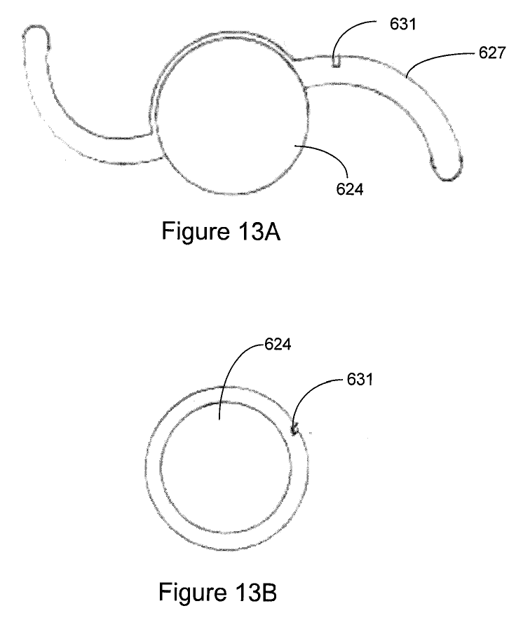

FIG. 13A shows a posterior chamber attenuation device in the shape of an intraocular lens with a filling valve 631 in the haptics 627 or, as depicted in FIG. 13B a filling valve 631 is located next to the lens 624 itself.

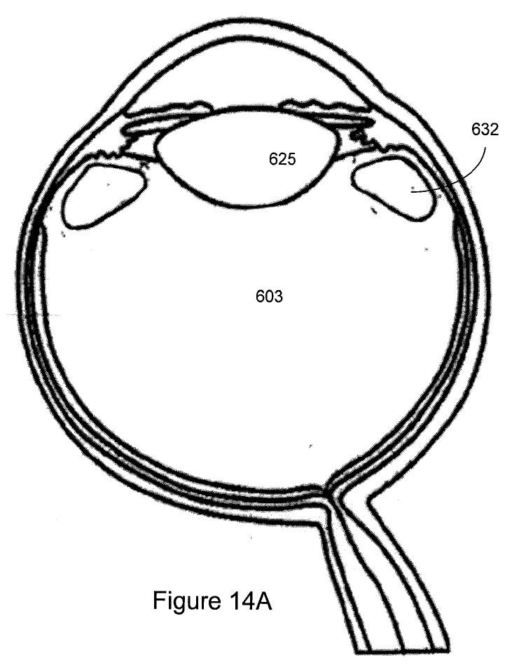

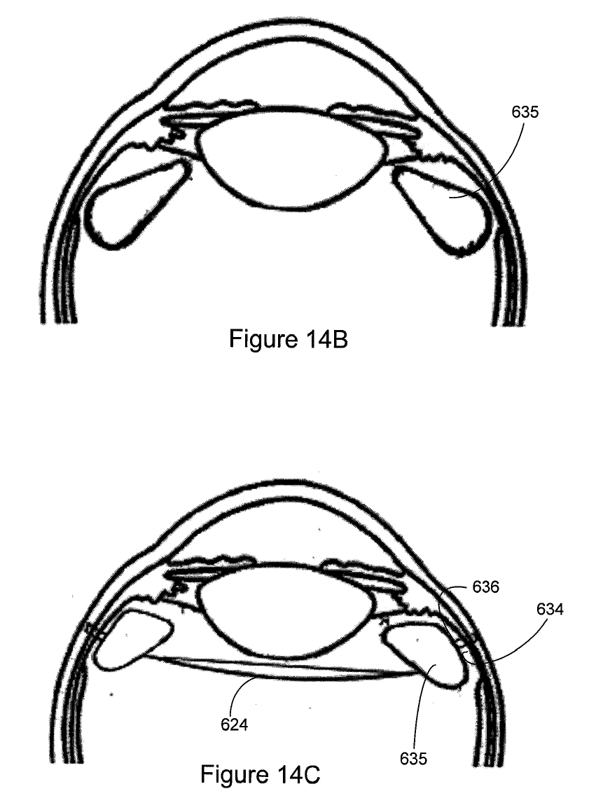

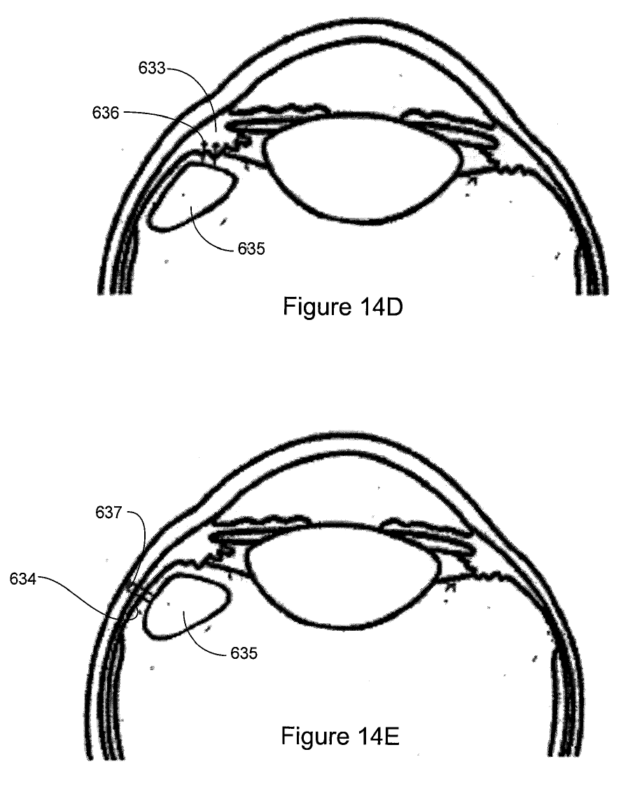

FIG. 14A shows a cross-section of an attenuation device 632 placed into the vitreous humor 603. In this embodiment the attenuator 632 is placed just posterior to the capsular bag 625 without interrupting the optical path. In another embodiment, the attenuation device is placed without creating a void in the vitreous humor. In another embodiment the device is placed after creating the appropriate void in the vitreous humor via a vitrectomy.

FIG. 14B shows a cross-section of an attenuation device 635 which is free floating in the vitreous, and angled upward at about 45 degrees. It does not enter the optical path. Shapes of the device may be similar to those demonstrated on FIG. 10C through 10H. The device is inserted into the vitreous via the same approach used in a trans-pars plana vitrectomy. The device 635 may reside just posterior to the capsular bag 625 as shown in 14B or in other locations more posterior in the vitreous chamber 603, including but not limited to near the optic nerve. FIG. 14C shows a cross-section of a device 635 that is anchored to the wall 634 of the vitreous humor 603. The anchor 636 could either be a valve mechanism to the attenuator 635 or simply a distinct anchor that secures the device. FIG. 14C also incorporates a lens 624 which may be either a zero optical power lens or visually enhancing lens.

FIGS. 14D and 14E show different configurations and anchors of attenuation devices that have been placed into the vitreous humor. FIG. 14D shows a one-sided attenuator 635 anchored or connected to the ciliary bodies 633 by means of a suture, staple, clip or other anchors 636 known to those skilled in the art. FIG. 14E shows a one-sided attenuator 635 anchored or connected to the wall 634 of the vitreous humor by means of either a valve mechanism or a distinct anchor 637.

Placement of the device into the vitreous is similar to other devices that are placed into the vitreous 603, such as U.S. Pat. No. 6,719,750. A pressure stabilizing or attenuating device may be inserted into the vitreous chamber 603 and could include the following steps:
Creating an incision in the conjunctiva
Inserting a cannula—0.25 mm-5 mm, more preferably 0.5 mm to 3.0 mm, more preferably 1.0 to 2.0 mm—that goes through the sclera into the vitreous.
Removing an appropriate volume of vitreous humour, if necessary, from about 0 cc to 3.0 cc, more preferably 0.25 cc to 1.5 cc or more preferably about 1 cc. Alternatively, vitreous humour may be withdrawn through a 20-25 gauge needle.
Inserting a delivery sheath into vitreous through the puncture site and dilating the access site to accommodate a 15-20 gauge sheath.
Inserting a delivery tube into the sheath and into vitreous chamber. Delivery tube shall be between about 0.5 mm and 5 mm, more preferably between about 1 and 3 mm.
Inflating the pressure attenuator to a volume from 0.01 cc to 5 cc, more preferably from 0.25 cc to 1.5 cc.

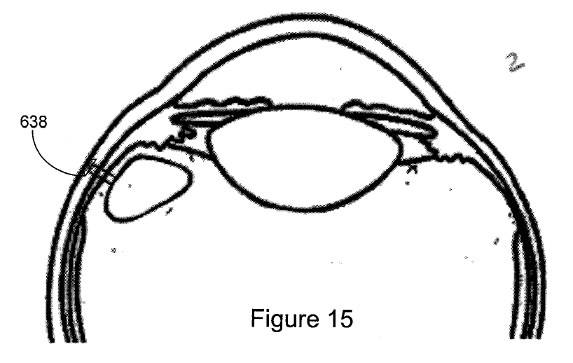

If the pressure attenuator needs to be anchored, one embodiment, as depicted in FIG. 10D and FIG. 15 could include attaching a cap 638 to the attenuator which affixes to the device, but sits outside the sclera and under the conjunctiva. A portion of the attenuator passes through the insertion site and a cap screws or snaps onto the device. The cap could be made of metal, such as stainless steel, titanium, various alloys, nitinol, cobalt chromium or any kind of polymer, resorbable or non-resorbable. The part of the device that the cap snaps onto could be metal, such as those listed above, or polymer. The cap prevents that attenuator from migrating into the vitreous and could facilitate removal or re-filling. Other methods of anchoring could include sutures, rivets, cords, staples or other anchors known to those knowledgeable in the industry.

Alternatively, the attenuation device could include a thin, pliable safety tether long enough to extend from the attenuation device and exit from the eye. The tether can be constructed of accepted materials such as those used in the manufacture of sutures, catheters and may also possess anti-microbial properties. In one embodiment, the distal end of the tether may be terminated with a lightweight pendant of sufficient bulk to prevent ingress of the entire tether into the eye. During normal use, the pendant may be temporarily affixed to the sclera. The tether may be used to remove or deconstruct the attenuation device.

Referring to FIGS. 16A through 16D, there is illustrated a variety of shapes for the attenuation device 66 in the form of an inflatable container. As illustrated, the devices used in embodiments described herein may take many shapes. Certain forms may provide better performance, in particular for providing baffling of pressure waves as well as attenuation of pressure spikes. Possible shapes for the attenuation devices include toroid like shapes, similar in form but not size to donuts and inner tubes; spoked wheel forms; horseshoe-like forms; mushroom-like forms; and banana-like forms.

FIG. 16A illustrates a toroidal embodiment, in which a plurality of central spokes is provided. FIG. 16B illustrates a crescent or “C” shaped attenuation device. Any of a variety of spherical, oval, elliptical or other shapes may be utilized such as those illustrated in FIG. 16C, in which the greatest length dimension of the inflated attenuation device is within the range of from about 1 to about 5 times the smallest cross-section. FIG. 16D illustrates a less arcuate variety as shown in FIG. 16B. In general, the attenuation device 66 may take any of a variety of forms which provides a sufficient volume to achieve the desired attenuation function, and which will minimize or eliminate risk of loss or obstructing outflow through the trabecular meshwork and other drainage ports in the eye.

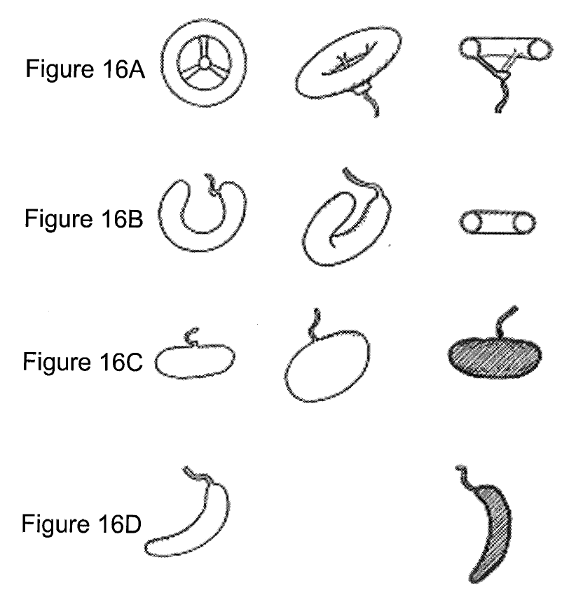

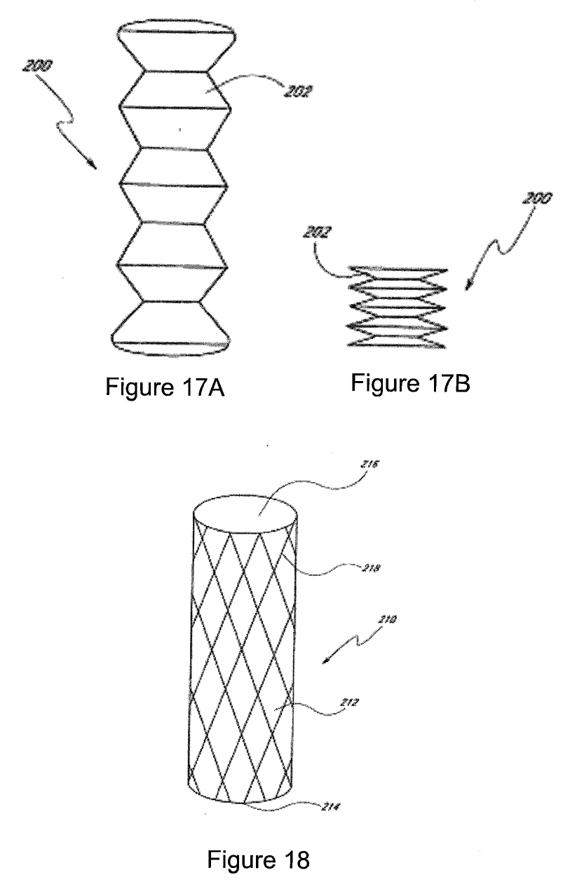

Referring to FIGS. 17A and 17B, there is illustrated an axially-compressible mechanical bellows type attenuation device. Attenuation device embodiments for absorbing transient pressure changes include diaphragmatic structures, rigid structures both shape changing and rigid with a coating or a bellows or bellows-like structure that can dampen pressure waves in the eye as a stand alone attenuation devices or as part of the wall or structure of the eye. One embodiment of a mechanically assisted attenuation device is in FIGS. 17A and 17B. FIG. 17A is a mechanical bellows that is in a normally extended position. The pressure within the bellows is reduced such that the bellows normally retains its extended position, but will compress when external pressure is exerted on it. The bellows could be made from plastic or metal, such as, for example, titanium or stainless steel from Senior Flextronics, Inc. Sharon, Mass. The bellows may be sealed, or covered in a material that allows for the reduction of air pressure within the structure.

This approach has the advantage for significantly greater change of volume with change of pressure. The theoretical limits of the air cell described herein can only be reduced approximately 25% of its volume, but this bellows system can contract to almost 90% of its volume.

The bellow attenuation device 200 comprises a membrane 202, which is collapsible in an accordion fashion. The membrane 202 may be self-supporting, or may be provided with an internal or external frame. The frame may comprise any of a variety of structures, such as a simple spring aligned in parallel with the longitudinal axis of the bellow, or pivotably moveable structures such as an axially compressible wire pantograph as will be understood in the art.

Referring to FIG. 18, there is illustrated a mechanically-assisted attenuation device 210. In this embodiment, a compressible tubular wall 212 having closed ends 214, 216 is supported by a self-expanding tubular frame 218. Any of a variety of self-expanding tubular or spherical frame structures may be utilized, such as “zigzag” wire frames well known in the abdominal aortic aneurysm graft arts. Although the abdominal aortic aneurysm graft application generally requires a relatively high, radially outwardly directed force, the present application would preferably be compressible with a relatively low compressive force (i.e., low radial force). This may be accomplished by using wires of smaller gauge, less wire per graft, leaving adjacent apexes unconnected to each other, or other technique to reduce the radial force of the wire cage. The wire cage or other support structure is preferably surrounded by a water impermeable membrane such as a balloon. Pressure within such balloon may be lower than 30 mmHg.

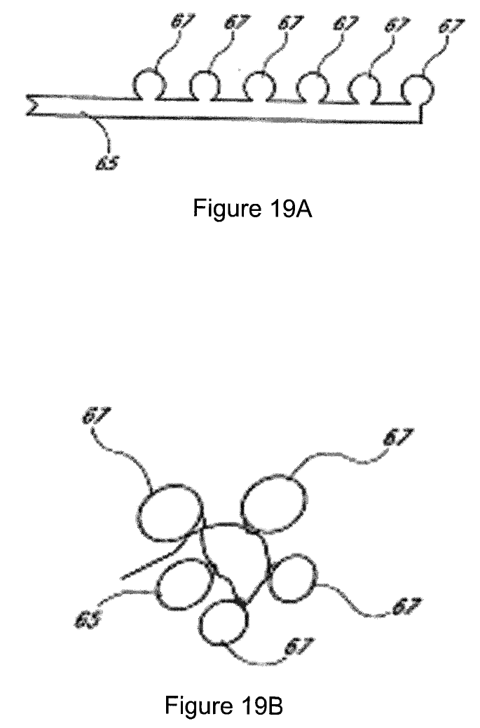

Referring to FIGS. 19A and 19B, there is illustrated another layout for the inflatable attenuation device 66. In this embodiment, illustrated in FIG. 19A, a plurality of attenuation devices 67 are connected by a common flow path 65, so that the plurality of attenuation devices 67 can be inflated through a single fill port. In another embodiment, illustrated in FIG. 19B, a plurality of self-expanding attenuation devices are connected by a suture, Nitinol wire, or other tether, thereby minimizing the crossing profile and/or maintaining a constant crossing profile for an attenuation device of any desired total inflated volume.

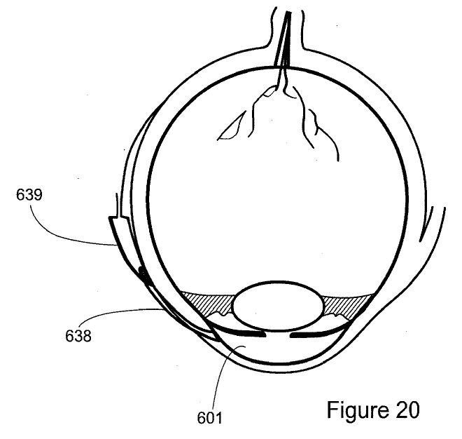

FIG. 20 shows another illustrative embodiment as an attenuation device that is part of drainage tube. The tube portion of the shunt 638 is placed in the anterior chamber 601, with the attenuating air cell 639 corresponding to the drainage port that lies beneath the sclera. The attenuator could either act in concert with the drainage tube, or by itself. For instance, a combination drainage tube and shunt might have a distal portion that is divided into two chambers, one serving as a drainage facility and the other serving as a gas-filled attenuation chamber. The proximal portion would be a tube that would sit in the anterior chamber of the eye. Changes in intraocular pressure would force fluid into the drainage tube 638 and through the drainage port. More rapid, dynamic changes in pressure would be attenuated by the attenuation chamber 639. In another embodiment the entire distal portion of the device is a gas-filled attenuation chamber which is in fluidic communication with the anterior chamber of the eye via a tube. Rapid changes in pressure get absorbed by the attenuation chamber. Placement of the shunt/attenuator combination device or the combination device may be subconjunctival and epi-scleral and could include the following steps:
Creating an incision through the conjunctiva and Tenon's capsule.
Forming a pocket at the superior quadrant between the medial or lateral rectus muscles by blunt dissection of Tenon's capsule from the episclera.
Inserting a compliant attenuation device or attenuation/shunt device into the pocket between the rectus muscles and sutured to the episclera. The leading edge of the device should be at least 8-10 mm from the limbus.
Trimming the drainage tube to allow 2-3 mm length into the anterior chamber. The tube preferably has a bevel cut to an anterior angle of 15-90 degrees.
Creating a paracentesis and entering the anterior chamber at the limbus with a sharp 23 gauge needle parallel to the iris.
Inserting the drainage tube into the anterior chamber approximately 2-3 mm through the needle track and parallel to the iris.
Covering the exposed attenuation device with a small piece of preserved donor sclera or pericardium which is sutured into place and the conjunctiva is closed.

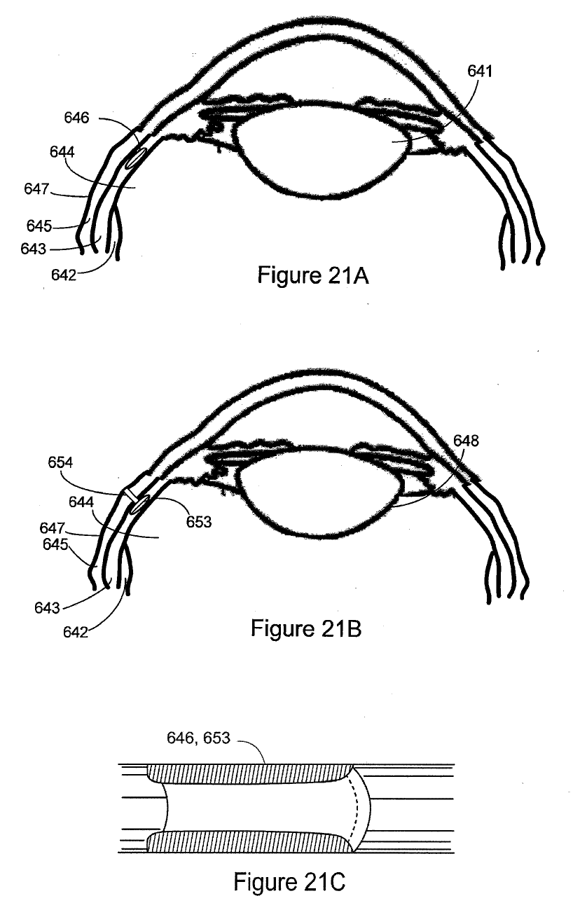

Another embodiment comprises an attenuator that is placed into the choroidal space. Other devices have been implanted into the choroidal space (see U.S. Pat. Nos. 5,766,242 and 5,443,505). These patents describe drug delivery devices that sit in an avascular region of the choroid—often the pars plana region. The choroid is a vascular bed of tissue that sits between the retina and the sclera of the eye. In young, healthy patients, this bed of tissue is compliant and can attenuate pressure waves. As people age, the choroid can calcify and become more rigid, reducing compliance. The location of an attenuator implanted in the choroidal or suprachoroidal space is illustrated in FIGS. 21A and 21B. As depicted in FIG. 21A, the attenuator 646 is implanted in the choroidal space 643 which sits between the retina 642 and the sclera 645. The device is deployed in the pars plana region 644 which is an avascular region of the choroids.

A similar device is shown in FIG. 21B, but with a valve 654 that extends from the attenuator 653 in the choroid 643 into the sclera 645. The valve 654 may be accessed post-operatively to enable re-filling the attenuator 653 with gas. FIG. 21C provides an illustrative embodiment of the attenuators 646, 653 shown in FIGS. 21A and 21B. As depicted, the attenuators 646, 653 are tubular in shape.

The procedure to position the attenuator within the choroid may include the following steps:
Creating incision in the conjunctiva 647
Creating a needlestick—0.25 mm-5 mm, more preferably 0.5 mm to 3.0 mm, more preferably 1.0 to 2.0 mm—through the pars plana region 644 of the sclera 645 and into the choroid 643.
Deploying the attenuator 646, 653 into the choroidal space 643.
Inflating the attenuator 646, 653 from 0.1 cc to 5 cc, more preferably from 0.25 cc to 1.5 cc.

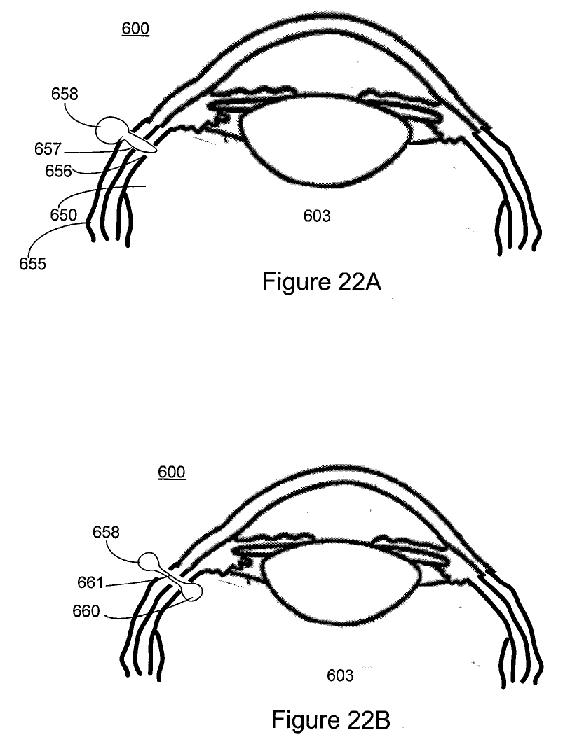

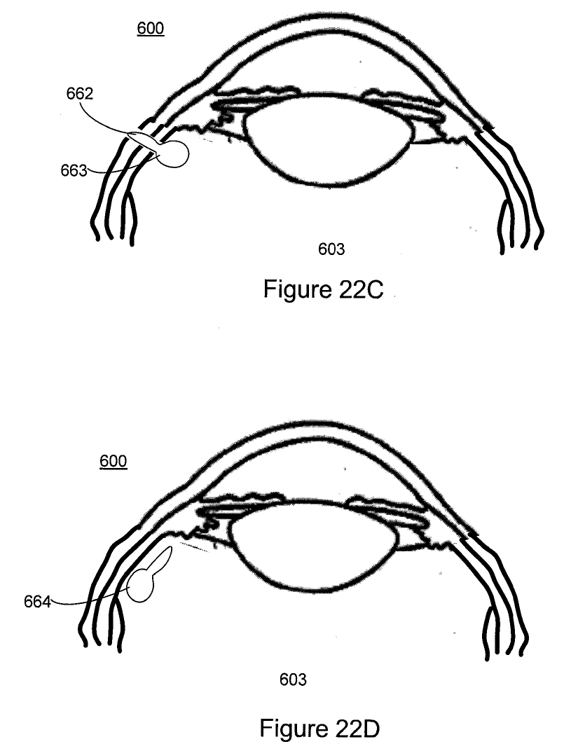

FIGS. 22A through 22D illustrate the insertion of an attenuator into the eye in an inflated state. In this embodiment, the device does not have a valve, and is configured such that one end of the device has a diameter that is smaller than the other end of the device. FIG. 22A shows the insertion of the attenuator 658, in an inflated state, such that the smaller diameter portion 657 of the attenuator 658 is inserted first through an incision 656 in the eye 600. Preferably, the incision is from 0.25 mm-5 mm, more preferably 0.5 mm to 3.0 mm, more preferably 1.0 to 2.0 mm through the pars plana region 650 of the sclera 655.

As shown in FIG. 22B, the attenuator 658 advances through the incision, the gas in the attenuator 658 shifts to the segment of the attenuator 660 that is within the eye 600. The diameter of the segment of the balloon 661 that is passing through the incision remains relatively constant as gas passes from one end of the attenuator 658 to the other. FIG. 22C shows the proximal end 662 (now the smaller diameter segment) passing through the incision, with the gas having shifted to the segment of the attenuator 663 now located within the eye 600. FIG. 22D shows the attenuator 664 fully deployed within the eye 600 in an unanchored state. The device 664 can either be anchored or unanchored.

In an alternative embodiment, a spherical attenuator has a chamber sealed with gas and perfluorocarbon. The attenuator is preferably compressed into an appropriate sized delivery sheath for delivery into the eye. As the device is deployed into the eye, it may return to its initial, uncompressed state.

One embodiment provided herein relates to the delivery of a very flexible, thin walled device. Delivery of an attenuation device is typically accomplished via a suitably sized introducer or possibly through the working channel of an opthalmoscope. However, in certain instances the columnar strength of an attenuation device may make it difficult to be pushed through such channels. A further requirement of any delivery system is that it be atraumatic, and not pose a threat of tissue damage. The embodiments described below address such issues, and offer improvements for accomplishing delivery of such attenuation devices as disclosed in co-pending applications U.S. Application Ser. No. 60/197,095, filed Apr. 14, 2000, titled Devices And Methods For Eye Pressure Attenuation, and U.S. application Ser. No. 09/723,309, filed Nov. 27, 2000, titled Devices And Methods For Attenuation Of Pressure Waves In The Body.

The attenuation device is normally folded on itself along its diameter in order to present a low profile for insertion into, for example, a patient's eye trans-sclerally. In this configuration the attenuation device has insufficient column strength to withstand the forces of insertion without buckling. If the attenuation device buckles it cannot be inserted. Following insertion the attenuation device is inflated via an inflation tube to which it is pre-mounted. After inflating the inflation tube is detached and the attenuation device is freed. By way of illustration, various embodiments are described in the exemplary context of trans-sclerally insertion of a delivery system into a patient's eye.

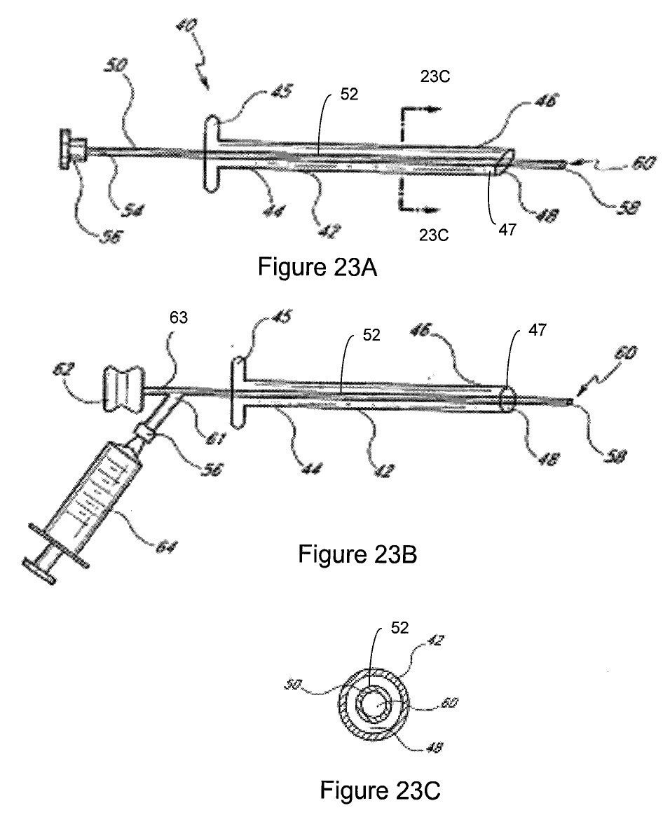

Referring to FIG. 23A, there is illustrated one delivery system for deploying the attenuation device into the treatment site within the eye. In general, the delivery system 40 is designed to advance an attenuation device 66 (not illustrated) trans-sclerally into the eye while in a first, reduced cross-sectional configuration, and to thereafter inflate or enlarge or permit the expansion of the attenuation device to a second, implanted orientation. The particular configuration and functionality of the delivery system 40 will therefore be governed in large part by the particular design of the attenuation device 66. Thus, as will be apparent to those of skill in the art in view of the disclosure herein, various modifications and adaptations may become desirable to the particular delivery system disclosed herein, depending upon the construction of the corresponding attenuation device.

The delivery system 40 comprises an elongate tubular body 42 having a proximal end 44 and a distal end 46. Tubular body 42 is dimensioned to trans-sclerally access the eye. Thus, the tubular body 42 preferably has an outside diameter of no more than about 5 mm, and, preferably, no more than about 3 mm. The length of the tubular body 42 may be varied, depending upon the desired proximal extension of the delivery system 42 from the eye during deployment. In general, an axial length of tubular body 42 within the range of from about 1″ to about 10″ is currently contemplated.

The tubular body 42 is provided with at least one central lumen 48 extending axially there through. Central lumen 48 axially slideably receives a filling tube 50, for filling the attenuation device 66. Filling tube 50 comprises a tubular body 52 having a proximal end 54 and a distal end 58. An inflation lumen 60 extends throughout the length of the tubular body 52, and is in fluid communication with a proximal hub 56. Hub 56 comprises a connector such as a standard leuer connector for coupling to a source of inflation media.

The tubular body 52 has an axial length which is sufficiently longer than the axial length of tubular body 42 to allow the proximal hub 56 to remain accessible to the clinician and accomplish the functions of deploying and filling the attenuation device 66. In one embodiment, an outer tubular sheath (not illustrated) is slideably carried over the tubular body 42, and is spaced radially apart from the tubular body 52 to define an annular cavity for receiving a rolled attenuation device 66 therein. In this manner, the deflated attenuation device can be rolled around a distal portion of the tubular body 52 and carried within the tubular sheath during trans-scleral placement. Once the delivery system 40 has been properly positioned, proximal retraction of the outer sheath with respect to the tubular body 52 exposes the deflated attenuation device 66. A source of inflation media is coupled to the proximal hub 56, and media is introduced distally through central lumen 60 to inflate the attenuation device 66. Following inflation of the attenuation device 66, the delivery system 40 is disengaged from the attenuation device 66, such as by retracting the filling tube 50 with respect to the tubular body 42. A distal stop surface 47 on tubular body 42 prevents proximal movement of the attenuation device 66 as the filling tube 50 is proximally retracted. Delivery system 40 is thereafter removed from the patient, leaving the inflated attenuation device 66 within the eye.

Biocompatible lubricating substances may be used to facilitate the placement of the attenuation device/fill tube within the lumen of the introducer. The distal tip of the introducer has been modified to allow a minimally traumatic presentation of the attenuation device to the eye tissue. Biocompatible lubricating substances may be used to facilitate the insertion of the attenuation device into the eye.

In one embodiment, the attenuation device incorporates biocompatible coatings or fillers to minimize irritation to the eye wall and/or to inhibit the formation of mineral deposits (encrustation). The materials can be coated onto the surface or incorporated within the wall of the attenuation device.

With reference to FIGS. 23B and 23C, there is illustrated a modified version of the delivery system 40. In this embodiment, a control 62 is connected by way of a proximal extension 63 to the tubular body 52. The control 62 may be in any of a variety of forms, such as a knob or a pistol grip. The control 62 may be grasped by the clinician, and utilized to axially advance or retract the filling tube 50 within the tubular body 42. The proximal hub 56 is connected to the tubular body 52 by way of a bifurcation 61. As will be appreciated by those of skill in the art, the central lumen 60 extends through the bifurcation 61 and to the proximal hub 56. Proximal extension 63 may comprise a blocked tubular element or a solid element. An inflation source 64 such as a syringe filled with a predetermined volume of air or other media may be connected to the proximal hub 56.

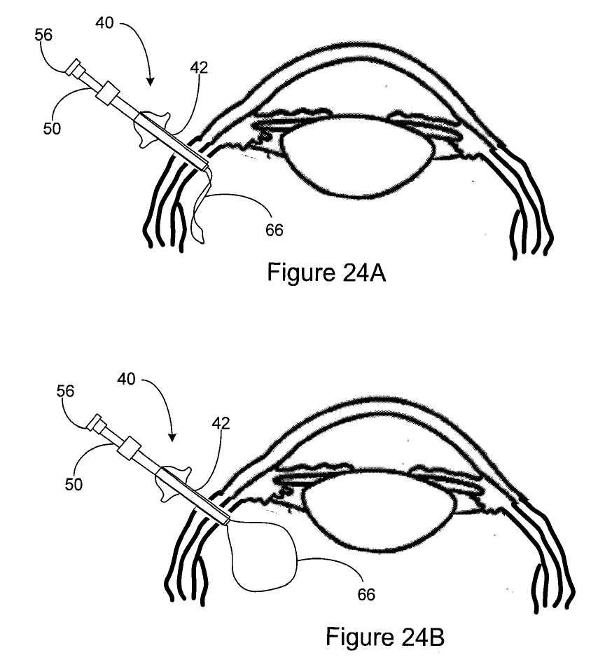

For patient comfort, the introducer is suitably sized to easily pass through the sclera into the eye (approximately 0.5 to 4 mm diameter). Visual feedback is provided to the clinician by means of insertion depth indicators along the longitudinal length of the introducer. The introducer may also have an adjustable depth stop that allows the clinician to pre-set the desired insertion depth. Once the delivery system has been inserted into the eye to the desired depth the introducer is then kept in a fixed position and the attenuation device mounted on the distal end of the fill tube is then extended in the lumen of the eye. The attenuation device is then filled with the indicated volume of gas from the attached syringe or similar device. See FIGS. 24A and 24B. Once properly inflated, the attenuation device is released from the fill tube using the tip of the introducer as an opposing force disengaging the attenuation device valve from the fill tube. The fill tube is then retracted completely into the lumen of the introducer and the entire delivery system is then withdrawn from the patient. The attenuation device is left in place for the clinically indicated period of time.

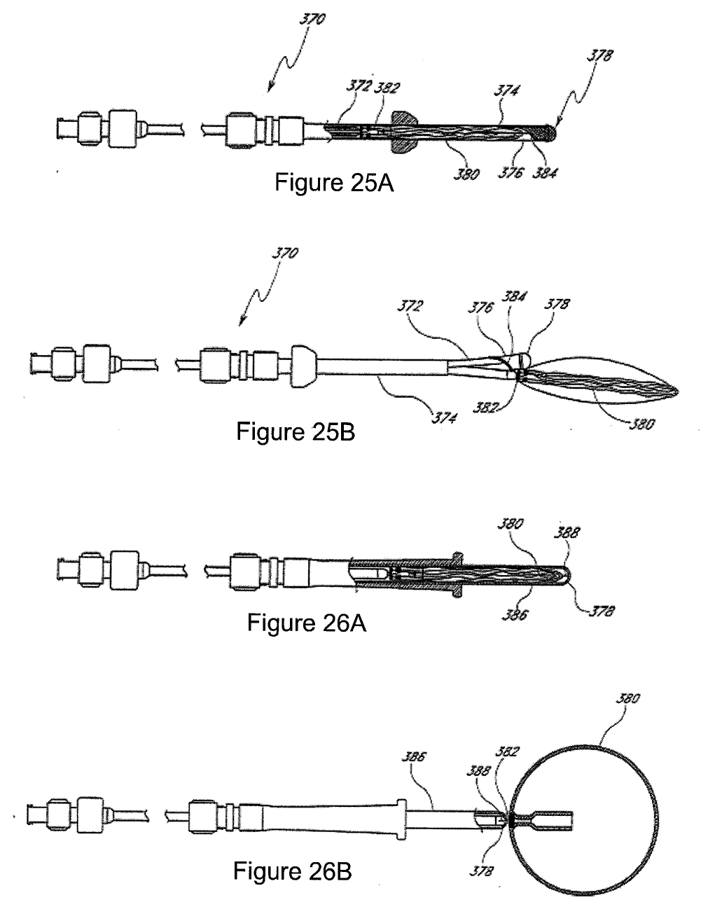

In another embodiment, shown in FIGS. 25A and 25B, there is provided a delivery system for the attenuation device which consists of an inner fenestrated tubular member which is provided with an atraumatic rounded tip at its distal end, and a slideably mounted outer coaxial tubular member. The rounded tip is shaped such that its proximal end, which is inserted into position in the distal end of the inner tubular member, presents essentially a “ramp” designed to aid ejection of the attenuation device from the fenestration when it is advanced. The attenuation device to be delivered is attached to its inflation tube, folded as previously described, and drawn into the inner sheath through the fenestration. Once situated within the fenestration the outer coaxial tubular member is slid forward to close the fenestration, thus containing the eye within the inner tube.

With reference to the embodiment illustrated in FIG. 25A, delivery system 370 comprises an inner sheath 372, a slideable outer sheath 374, an opening 376 in the inner sheath, and an atraumatic tip 378. With reference to the embodiment illustrated in FIG. 25B, delivery system 370 comprises an outer sheath 374 that slides backwards and an attenuation device 380. Here, the attenuation device 380 is exposed through the opening 376. The delivery system 370 comprises an inflation tube 382 that is advanced toward the atraumatic tip 378, thereby causing the attenuation device 380 to be ejected. A curved ramp 384 in the delivery system 370 aids the ejection of the attenuation device 380.

In use the distal end of the delivery system is inserted into the eye to an appropriate depth, the outer coaxial tube is slid backwards along the inner tube, thus exposing the fenestration in the inner tube. The attenuation device is advanced using the inflation tube and releases easily from the inner tube. The attenuation device is inflated, released from the inflation tube, and floats freely in the eye or, alternatively, can be tethered.

In another embodiment, shown in FIGS. 26A and 26B, the attenuation device containment tube 386 is a simple open-ended cylinder. The attenuation device 380 is folded as described previously and withdrawn into the containment tube 386. The open end of the containment tube 386 would present a potentially traumatic edge to the eye. In order to prevent such trauma, the open end of the containment tube 386 in this instance has a rounded atraumatic end 378. This end 378 contains slits 388 which, on sliding the containment tube 386 backwards allows the end 378 to open, thus allowing deployment of the attenuation device 380 from the containment tube 386. On advancing the inflation tube 382 with the attenuation device 380 attached, the slits 388 open and present little barrier to the deployment of the attenuation device.

In another embodiment, a removable delivery system is used to deliver, deploy, and fill the attenuation device. The delivery system can take the form of the system taught by U.S. Pat. No. 5,479,945, titled “Method And A Removable Device Which Can Be Used For The Self-Administered Treatment Of Urinary Tract Infections Or Other Disorders,” the disclosure of which is incorporated in its entirety herein by reference.

Suitable materials for the production of the attenuation devices include but are not limited to foldable or compressible materials, such as silicone polymers, hydrocarbon and fluorocarbon polymers, hydrogels, soft acrylic polymers, polyesters, polyamides, polyurethane, silicone polymers with hydrophilic monomer units, fluorine-containing polysiloxane elastomers and combinations thereof. It is preferred that attenuation device be of a bicomposite material design whereby optic and haptic elements are manufactured from a compressible or foldable material such as but not limited to a silicone or acrylic materials such as but not limited to copolymers of ethyl acrylate/ethyl methacrylate/trifluoroethyl methacrylate, phenylethyl acrylate/phenylethyl methacrylate, and other copolymers of acrylic esters suitable for a foldable refractive optic. Alternatively, the optic may be manufactured from a compressible or foldable material such as but not limited to a silicone or acrylic material, and the haptics and fixation clamps may be manufactured from a relatively more rigid material such as but not limited to a relatively more rigid hydrogel, PMMA or polyimide. Various acrylic copolymers are preferred for the manufacture of the optic portion of IOL due to its high refractive index of approximately 1.47-1.55, which is greater than that of the aqueous humor of the eye, i.e., 1.33.

The attenuation devices can be dip molded or extruded in a plurality of biocompatible materials. Furthermore, the attenuation devices can be fabricated from a variety of multi-layer composites or produced by a number of different manufacturing processes. Here, the designs of the attenuation devices are characterized by minimization and control of the gas and moisture vapor permeabilities in and out of the attenuation device.

The gas and moisture vapor permeabilities of any given material will vary depending on the conditions surrounding the material. For example, an attenuation device comprised of a certain material can have different gas and/or moisture permeabilities within the eye than at standard temperature and pressure. In addition to exposure to aqueous humor or vitreous humour, the intraocular environment includes exposure to pressure variations in the range of from about 10.0 mmHg to about 50 mmHg at rest or equilibrium, with transient pressure spikes ranging from 0.5 mmHg to as high as 30 mmHg or more. The body temperature is normally about 98 degrees F. or greater, and the attenuation device resides in 100% humidity. Long term efficacy of the attenuation device may be compromised if there exists any fluid or vapor exchange through the wall of the attenuation device in situ. The relative impermeability of the wall under normal intraocular conditions is preferably accomplished without losing the compliancy of the attenuation device which allows it to compress within the eye as is described elsewhere herein.

In general, the wall of the attenuation device will comprise at least one gas barrier layer and at least one moisture barrier layer. Any of a variety of gas barrier materials (e.g. polyvinylidene chloride, ethyl vinyl alcohol, fluoropolymers, etc.), available in thin film constructions, may be implemented into the attenuation device design. These materials are generally relatively stiff, have high moisture vapor permeability, and have low impact strength. Consequently, layering the film with flexible, high moisture barrier, high impact strength polymers is desirable. A variety of relatively flexible materials, having high moisture barrier characteristic and optionally high impact strength that can be formed into thin film sheets include but are not limited to: polyamide, polyethylene, polypropylene, polyurethane, polyamide/polyester copolymer, polystyrene/polybutadiene copolymer, etc. In one embodiment, at least one layer on, or the entire attenuation device comprises a blend of a barrier material and a flexible high impact strength material (e.g. polyurethane/polyvinylidene chloride, polyethylene/ethyl vinyl alcohol, etc.).

The attenuation device typically has two or more layers or barriers. For example, the attenuation device can have a gas barrier layer and a moisture barrier layer. An additional layer may be included to enhance the structural integrity of the attenuation device. In one embodiment, the attenuation device has an outer layer comprising a gas barrier and an inner layer comprising a moisture barrier. In another embodiment, the attenuation device has an outer layer comprising a moisture barrier and an inner layer comprising a gas barrier.

The attenuation device can have three, four, five, or more layers. In one embodiment, the attenuation device has a gas barrier layer, a moisture barrier layer, and one or more layers composed of at least one high impact strength material. In another embodiment, the attenuation device has multiple gas barrier layers arranged in a nonconsecutive arrangement. In yet another embodiment, the attenuation device has multiple moisture barrier layers arranged in a nonconsecutive arrangement. With respect to those embodiments having multiple, nonconsecutive barrier layers, the other layers of the attenuation device can include high impact strength material layers and/or other types of barrier layers.

The overall thickness of the wall is preferably minimized, and will often be no more than about 0.03 inches. Preferably, the wall will be no more than about 0.006 inches, and, in some implementations, is no more than about 0.001 inches thick. An outer layer may comprise a soft, conformable material such as polyurethane, EVA, PE, polypropylene, silicone or others, having a thickness within the range of from about 0.0025 inches to about 0.025 inches. The adjacent barrier layer may comprise EVOH, PVDC or other materials in a thin film such as from about 5 microns to about 25 or 30 microns thick. If the attenuation device is fabricated by bonding two sides together, a bonding or tie layer may be provided on the barrier layer. Tie layers comprising polyurethane, EVA or others may be used, having a thickness of preferably no greater than about 0.001 inches. Layers of less than about 0.0008 are preferred, and layer thicknesses on the order of from about 0.0003 to about 0.0005 inches are contemplated.

The layers of the attenuation device can be formed in any number of ways known to those skilled in the art, including, but not limited to, lamination, coextrusion, dip molding, spray molding, or the like, etc. As discussed above, the layers of the attenuation device can be formed from various materials. With respect to those attenuation devices that are formed by laminating two or more layers together, various different laminating techniques known to those skilled in art can be used, including, but not limited to, heating, solvents, adhesives, tie layers, or the like.

The material may not need to be elastomeric at all for the attenuation device to function. However, the materials chosen for use in embodiments of described herein are to be sufficiently flexible in the thickness ranges dictated by the selected designs. When the attenuation device is subjected to external pressures, the attenuation device's material is able to transmit the pressure to the contained air or pressure management construct and respond sacrificially as one of the most compliant members of the eye.

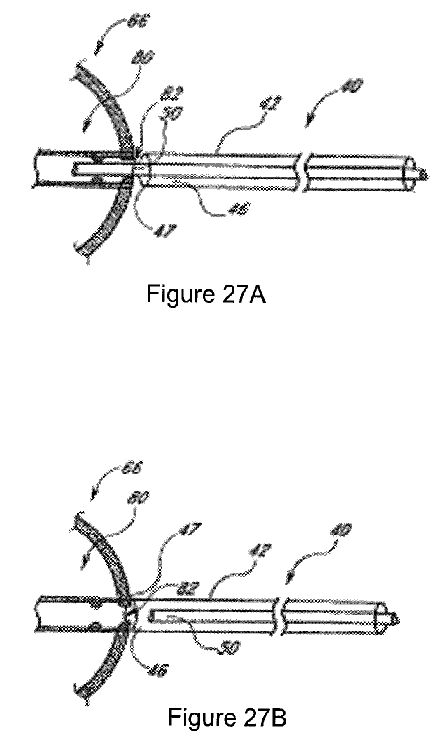

With reference to FIGS. 27A and 27B, there is illustrated one disengagement sequence for deploying the inflatable attenuation device 66 from the delivery system 40 described above. As illustrated in FIG. 27A, the delivery system 40 is initially configured with the filling tube 50 positioned within the valve 80. The distal end 46 of outer tubular body 42 is dimensioned such that it will not fit through the aperture 82 of valve 80. Once the attenuation device 66 has been positioned within the eye, the attenuation device 66 is inflated through filling tube 50.

With reference to FIG. 27B, the filling tube 50 is proximally retracted following inflation so that it disengages from the valve 80. This is accomplished by obstructing proximal movement of the attenuation device 66 by stop surface 47 on the distal end 46 of tubular body 42. The attenuation device 66 is thereafter fully disengaged from the delivery system 40, and the delivery system 40 may be removed.

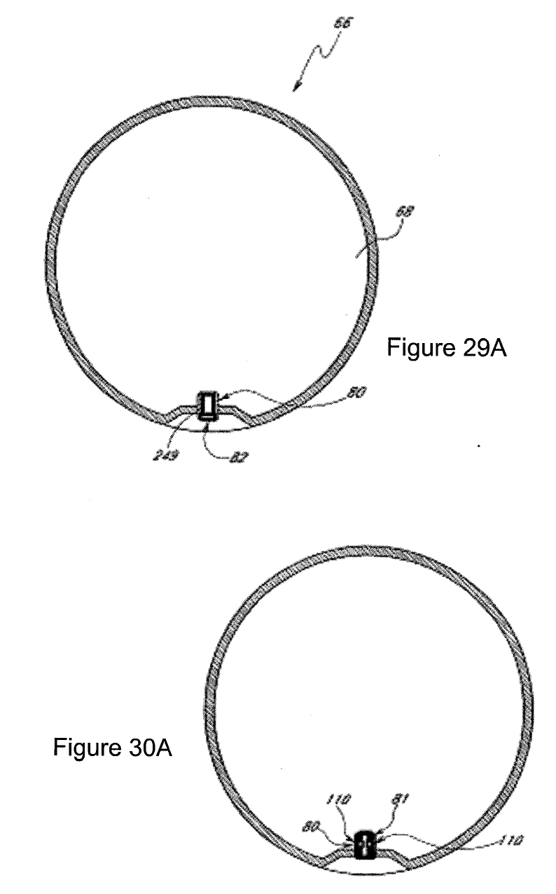

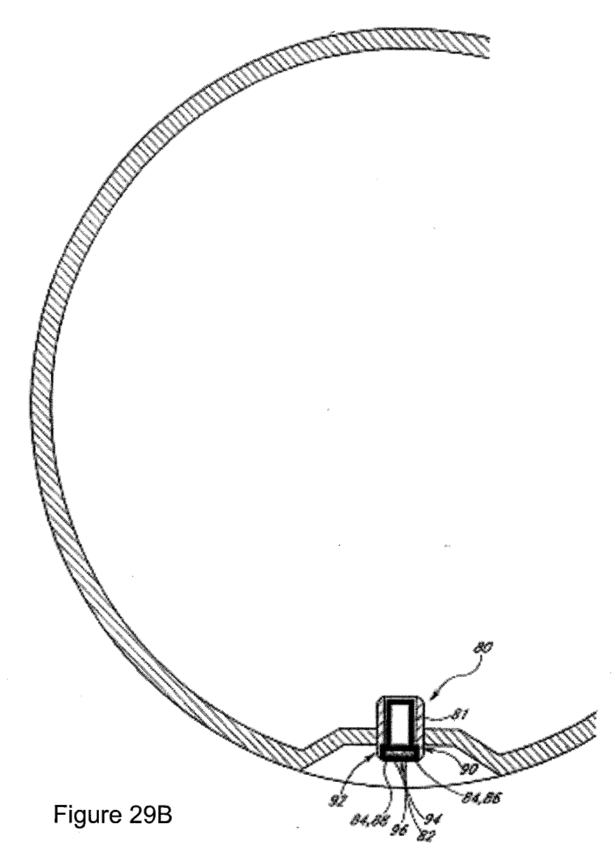

With reference to FIGS. 28A, 29A, and 29B, there is illustrated a duckbill embodiment of the valve 80. Valve 80 comprises a tubular wall 81, having an aperture 82 in communication with a flow path 83. At least one closure member 84 is attached to the tubular wall, and extends across the flow path 83. In the illustrated embodiment, closure member 84 comprises first and second duck bill valve leaflets 86 and 88 which are attached at lateral edges 90 and 92 to the tubular wall. The leaflets 86 and 88 incline medially in the distal direction to a pair of coaptive edges 94 and 96. This configuration allows forward flow through flow path 83 to separate coaptive edges 94 and 96, thereby enabling inflation of the attenuation device 66. Upon removal of the inflation media source, the inflation media within the attenuation device 66 in combination with natural bias of the leaflets 86 and 88 cause the leaflets to coapt, thereby preventing effluent flow of inflation media through the flow path 83.

The tubular body 81 and first and second leaflets 86 and 88 may be manufactured from any of a variety of materials which will be apparent to those of skill in the art. For example, tubular body 81 may be made from polyurethane such as by extrusion. Leaflets 86 and 88 may be made from any of a variety of flexible materials such as polyurethane, silicone, or polyethylene, and may be bonded to the tubular element 81 using adhesives, heat bonding, or other bonding techniques known in the art. Suitable valves include the valve manufactured by Target Therapeutics and sold as the DSB silicon balloon to fill aneurysms and arterial-venous malformations.

With continued reference to FIGS. 28A, 29A, and 29B, in one method of manufacturing the attenuation device 66, the bushing 249 is RF welded to the inflatable container 68 prior to installing the valve 80. Here, the duckbill valve 80 is bonded to the bushing 249 after welding. In one method of manufacturing the attenuation device 66, the mandrel is installed during welding, resulting in a polished surface with an air-tight seal along the inside of the tube.

Referring to FIG. 28B, closure is accomplished by two coaptive edges on distal end 106 of tubular body 81. This construction is sometimes referred to as a flapper valve. The tubular body 81 in this embodiment is formed by a first wall 96 and a second wall 100 which are bonded or folded along a first edge 102 and a second edge 104 to define a flow path 83 extending there through. The free distal ends of first and second walls 96 and 100 at the distal end 106 form coaptive leaflets, which may be opened under forward flow pressure through the flow path 83 and will inhibit or prevent reverse flow through the flow path 83.

Referring to FIG. 28C, the proximal end of the flow path 83 on the flapper valve of FIG. 28B or other valve structure may be reinforced such as by a reinforcing tube 108. Reinforcing tube 108 may be manufactured in any of a variety of ways. For example, reinforcing tube 108 may be extruded from various densities of polyethylene, Pebax, polyurethane, or other materials known in the art. Reinforcing tube 108 may be desired to maintain patency of the pathway to the valve 80, particularly in an embodiment adapted for coupling to a deflation and removal system as will be discussed. In another embodiment, the reinforcing tube 108 may be removable and used to prevent sealing of the valve during the manufacturing process and may also ease the placement of a fill tube in the valve. This reinforcing tube 108 is removed after the manufacturing process is complete, or may be removed before, during, or after the fill tube is placed.

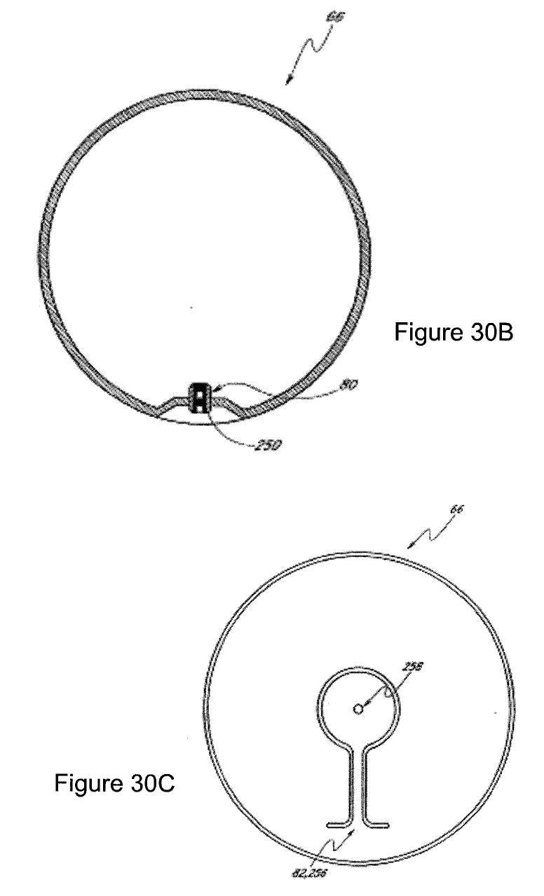

With reference to FIGS. 28D and 30A, there is illustrated an additional feature that may additionally be incorporated into any of the valves discussed above. In one embodiment of this feature, an annular sealing ring 110 is provided on the interior surface of the tubular body 81. Annular sealing ring 110 is adapted to provide a seal with the filling tube 50, to optimize the filling performance of the attenuation device. Sealing ring 110 is thus preferably formed from a resilient material such as silicone or polyurethane and dimensioned to slideably receive the filling tube 50 there through. In another embodiment, sealing with the fill tube may be enhanced by restricting the aperture diameter without the use of a distinct sealing ring 110.

With reference to FIGS. 28E and 30C, the valve may also be placed in the body of the attenuation device, rather than in the seam. In one exemplary embodiment, the through hole 258 has a diameter of 0.062 inches. Here, the inflation channel 256 has a diameter of approximately 0.063 to 0.070 inches. The valve can be placed in any number of ways including the methods described in U.S. Pat. No. 5,248,275, titled Balloon with flat film valve and method of manufacture, issued Sep. 28, 1993, and U.S. Pat. No. 5,830,780, titled Self-closing valve structure, issued Nov. 3, 1998; both of these patents are hereby incorporated by reference herein and made a part of this specification.

In one embodiment, shown in FIG. 30B, the valve 80 has a fill/plug 250. In one method of manufacturing the fill/plug attenuation device 66, the mandrel is installed during welding, resulting in a polished surface with an air-tight seal along the inside of the tube.

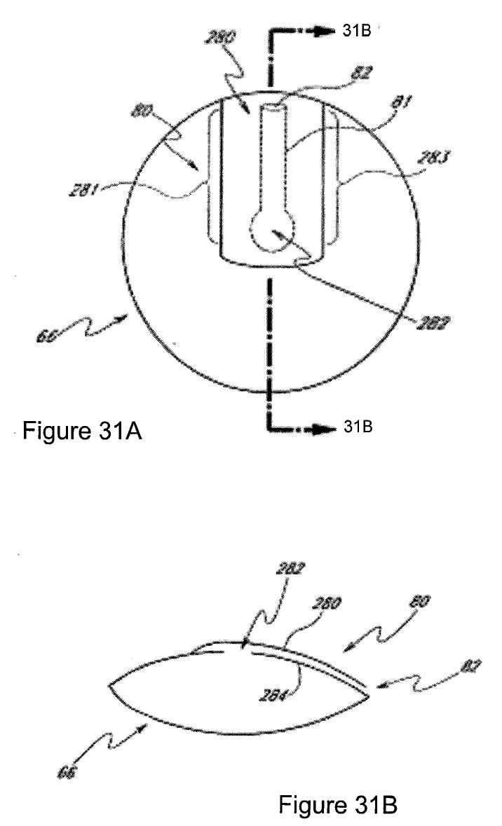

In another embodiment, the compressible attenuation device is provided with a valve that permits filling of the attenuation device through a filling device and yet resists deflation and/or additional filling of the attenuation device after the filling device is removed. In one embodiment, illustrated in FIGS. 31A and 31B, the valve 80 is formed by two parallel welds 281, 283 at the interface between two complimentary surfaces—namely, the outer cover 280 and the underlying layer 284. The valve 80 is in effect a collapsible airflow passageway that remains in the collapsed position when the filling device is removed, thereby preventing deflation when the pressure within the attenuation device 66 is greater than the pressure immediately outside the attenuation device and preventing the additional filling of the attenuation device 66 when external pressure is greater than the pressure within the attenuation device 66. The outer cover 280 and the underlying layer 284 function as two flat sheets that stick together regardless of the relationship between the internal attenuation device pressure and the immediate external pressure. In one embodiment (not shown), one or more adhesive materials or general locking mechanisms known in the art of medical device design can be used to shut the value 80 upon removal of the filling device. It should be noted that once the filling device enters the valve at the entry point 82, the attenuation device can be released and/or filled at any point inside of the entry point 82, including but not limited to the interface 282 between the valve 80 and the inside of the attenuation device 66. The valve of the present embodiment can be constructed according to the disclosure provided by U.S. Pat. No. 5,144,708, titled check valve for fluid eyes, issued Sep. 8, 1992, the disclosure of which is incorporated in its entirety herein by reference.

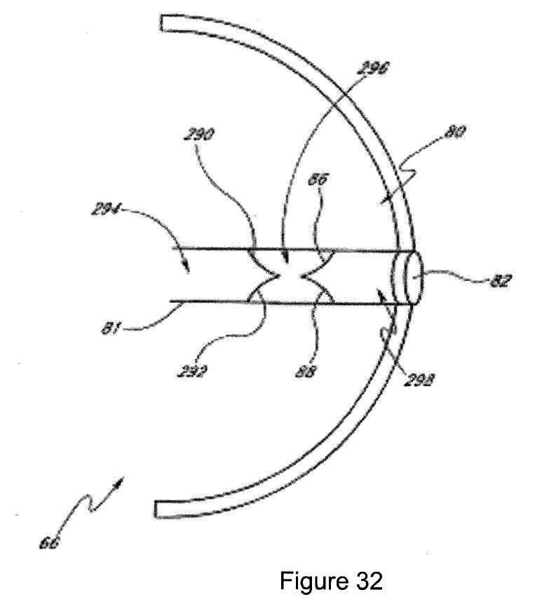

In another embodiment, illustrated in FIG. 32, the valve 80 includes two duckbill structures that face opposite each other, thereby permitting filling of the attenuation device through a filling device while resisting deflation and/or additional filling of the attenuation device after the filling device is removed. The valve 80 generally comprises a tubular wall 81, having an aperture 82 in communication with a flow path 298. The valve has two sets of first and second duck bill valve leaflets 86, 88, 290, 292 that are attached to the tubular wall 81. Upon removal of the inflation media source, the inflation media within attenuation device 66 in combination with natural bias of the leaflets 86 and 88 cause the leaflets to coapt, thereby preventing effluent flow of inflation media through the flow path 83. In addition, the natural bias of the leaflets 290 and 292 cause the leaflets to coapt, thereby preventing the additional influx of media. It should be noted that the internal section 294 of the tube will have a pressure equal to the internal pressure of the attenuation device, whereas the external portion or flow path 298 will have a pressure equal to the immediate external pressure. A middle or neutral section 296 of the tube is defined by the tubular wall and the two oppositely facing duckbill structures defined by leaflets 86, 88, 290, 292.

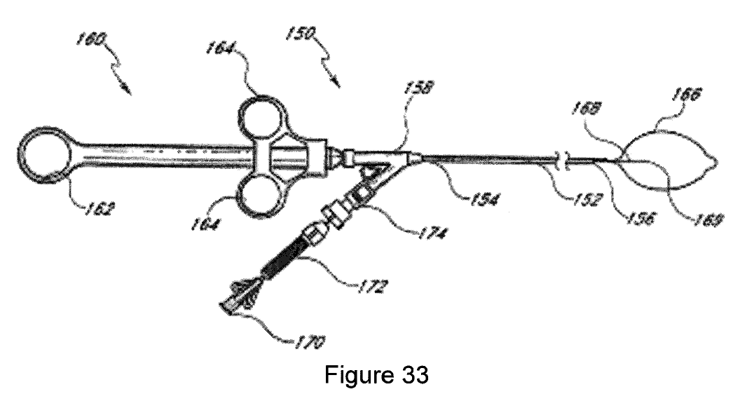

The attenuation device 66 is preferably also removable from the eye. Removal may be accomplished in any of a variety of ways, depending upon the construction of the attenuation device. Preferably, removal is accomplished trans-sclerally. In one embodiment, removal is accomplished by reducing the attenuation device 66 from its second enlarged profile to its first, reduced profile so that it may be withdrawn trans-sclerally by a removal system. The removal system will be configured differently depending upon whether reduction from the second profile to the first profile is accomplished by deflation, or by compression. One embodiment of a removal system utilized to remove an inflatable attenuation device 66 will be described below in connection with FIG. 33.

Another embodiment, however, provides a removal procedure that involves dissolving or degrading the material or a portion of the material of the attenuation device 66 in situ. Material selection and wall thickness of the attenuation device 66 may be optimized to provide the desired useful life of the attenuation device 66, followed by dissolution in the aqueous environment of the eye. In one embodiment, dissolution or deflation may be catalyzed or accelerated by an accelerating event such as a change in pH or introduction of an initiator or accelerator into the eye, or reduction of pressure.

Attenuation devices having a predetermined dwell time after which they are removed can be manufactured in a variety of ways through the use of bioabsorbable or permeable materials. In one embodiment, the entire wall of the inflatable container 68 is made from an absorbable material. As used herein “absorbable” means any material which will dissolve, degrade, absorb or otherwise dissipate, regardless of the chemical mechanism, to achieve the purpose recited herein. In another embodiment, only a portion of the flexible wall 70 or other portion of the attenuation device such as the valve is made from an absorbable material. As soon as one or more windows or “fuse” components of the attenuation device is absorbed, the attenuation device will deflate through the resulting opening and then can be removed. In yet another embodiment, one or more seams such as seam 78 can be bonded by a dissolvable or absorbable material that is designed to fail after a predetermined time in the aqueous environment of the eye.

The resulting deflated components from any of the foregoing time limited embodiments can remain in the eye in a deflated state until removed using a removal system. In one embodiment, the material or portion of the inflatable container 68 is made from a gas permeable material. In one embodiment, the attenuation device is filled with approximately 5 ml of gas and the attenuation device's material allows approximately 3.5 ml of gas to permeate out of the attenuation device over certain time intervals, such as, for example, one, three, six, or twelve months. Once the volume remaining is less than approximately 1.5 ml, the attenuation device is normally removed.

The predetermined dwell time within the eye can be influenced by a variety of design factors, including the formulation of the absorbable material and the physical shape, thickness and surface area of the absorbable component. A variety of absorbable polymers which can be used in the embodiments disclosed herein are known in the absorbable suture arts. For example, absorbable multifilament sutures such as DEXON sutures (made from glycolide homopolymer and commercially available from Davis & Geck, Danbury, Conn.), VICRYL sutures (made from a copolymer of glycolide and lactide and commercially available from Ethicon, Inc., Sommerville, N.J.), and POLYSORB sutures (also made from a copolymer of glycolide and lactide and commercially available from United States Surgical Corporation, Norwalk, Conn.) exemplify materials known in the industry and characterized as short term absorbable sutures. The classification short term absorbable sutures generally refers to surgical sutures which retain at least about 20 percent of their original strength at three weeks after implantation, with the suture mass being essentially absorbed in the body within about 60 to 90 days post implantation.

Certain bioabsorbable elastomers may also be used to form the attenuation devices or fuses. The elastomers can be melt-processed, for example by extrusion to prepare sheets, plugs or tubular structures. In one embodiment, the copolymers can be injection molded to fabricate intricately designed parts, or compression molded to prepare films. For the details of such melt-processing techniques, see, for example, F. Rodriguez, Principles of Polymer Systems, Chapter 12 (McGraw Hill 1970).

The bioabsorbable elastomers can also be solvent cast to prepare thin films. Solvent casting can be accomplished using conventional methods such as first dissolving the copolymer in a suitable solvent to make a solution, then casting the solution on a glass plate to make a film, and then evaporating the solvent from the cast film. In another processing scheme, the copolymers can be lyophilized to prepare foams. Lyophilization can be accomplished by first dissolving the copolymer in an appropriate solvent, freezing the solution, and then removing the solvent under vacuum. The set of appropriate solvents include p-dioxane. Lyophilization techniques to prepare films are described in Louis Rey, Aspects Theoriques Et Industriels De La Lyophilization (1964).

Certain bioabsorbable elastomers are disclosed in U.S. Pat. No. 6,113,624, titled Absorbable elastomeric polymer, issued Sep. 5, 2000, the disclosure of which is incorporated in its entirety herein by reference. In accordance with the process disclosed therein, a two-step, one-reaction vessel, two-temperature process is utilized in which a mixture of p-dioxanone monomer and p-dioxanone homopolymer, is formed at low temperatures of from about 100° C. to about 130° C., preferably 110° C. The mixture is then reacted with lactide at temperatures from about 120° C. to about 190° C. to form copolymers in which segments or sequences are composed of both p-dioxanone and lactide repeating units. These segmented copolymers are stated to be less crystalline than the block or graft copolymers previously known in the art and, therefore, yield materials with good strength, but shorter BSR (“Breaking Strength Retention”) profiles, faster absorption rates, much longer elongations and lower stiffness than the block copolymers. A wide variety of copolymers of polylactic and polyglycolic acids are also known in the art, particularly for use with absorbable orthopedic screws and fasteners.

The ideal material can be optimized through routine experimentation taking into account the attenuation device design and the desired indwelling time period. Attenuation devices may be time rated, such as 15 days, 30 days, 45 days, 90 days, 180 days or other as may be desired.

Referring to FIG. 33, there is illustrated a side elevational schematic view of one embodiment of an intraocular removal system. This removal system is adapted to retrieve the inflatable attenuation device discussed elsewhere herein. The removal system 150 comprises an elongate tubular body 152 which extends between a proximal end 154 and a distal end 156. Tubular body 152 is dimensioned to trans-sclerally access the eye. In one embodiment, the removal system 150 is adapted for use in conjunction with standard ophthalma-scopes, having minimum working channels of approximately 0.5 to 6.0 mm.

The tubular body 152 may be manufactured in accordance with any of a variety of techniques well understood in the catheter and other medical device manufacturing arts. In one embodiment, tubular body 152 is extruded from a biocompatible material such as TFE, having an inside diameter of approximately 0.09 inches and a wall thickness of about 0.01 inches.

The proximal end 154 of tubular body 152 is connected to a Y-adaptor 158. Y-adaptor 158 carries a control 160 for controlling the retrieval system as will be described. Control 160 in the illustrated embodiment comprises a thumb ring 162 which is slideably carried with respect to a pair of finger rings 164. Axial movement of the thumb ring 162 with respect to the finger rings 164 enlarges or retracts a retrieval loop 166 extending distally from distal end 156 of tubular body 152. Retrieval loop 166 is adapted to surround the inflated attenuation device 66. In one embodiment, the loop 166 has an enlarged diameter of about 5 mm, and comprises a wire such as 0.4 mm diameter stainless steel cable wire.

In use, the loop 166 is opened once the distal end 156 of the tubular body 152 has reached the eye. The loop 166 is positioned around the attenuation device 66, and the proximal control 160 is manipulated to tighten the loop 166 around the attenuation device 66. After the attenuation device 66 has been securely grasped by the loop 166, if the attenuation device 66 is not already deflated, a deflating tube 168, preferably having a sharpened distal tip 169 thereon, is distally advanced through the wall of the attenuation device 66. Distal advancement of the deflating tube 168 may be accomplished by distally advancing a proximal control, such as control 172. The distal tip 169 is in fluid communication with a connector such as a standard luer adaptor 170 through a central lumen (not illustrated), so that an empty syringe or other device may be connected to the connector 170 and used to evacuate the contents of the ensnared attenuation device 66. As the attenuation device 66 is deflated, the control 160 may be manipulated to pull the collapsed attenuation device 66 into the distal end 156 of the tubular body 152. The removal system 150 having the reduced attenuation device 66 therein or carried thereby may be trans-sclerally removed from the patient.

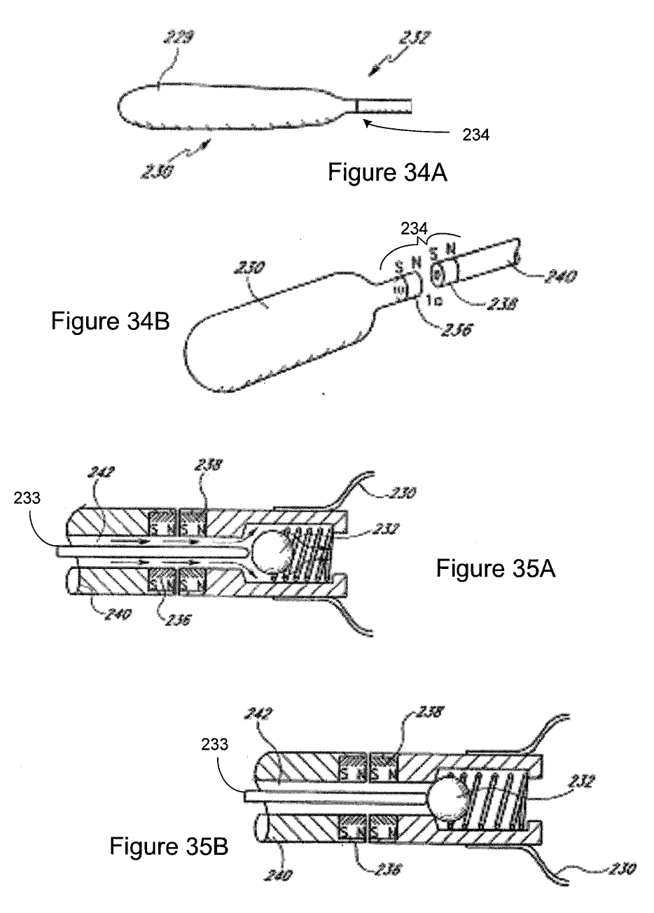

A wide variety of modifications can be made to the foregoing removal system 150. For example, the proximal controls 160 and 172 may be combined into a pistol grip or other configuration. Controller 172 or control 160 may additionally control deflection of the distal end 156 of the tubular body 152, or control rotation of the plane of the loop 166. In general, the removal system 150 preferably accomplishes the basic functions of enabling the location of the attenuation device 66, capturing the attenuation device, reducing the attenuation device in size and removing the attenuation device from the eye. The capturing step may be accomplished by visualizing the attenuation device through the opthalmoscope, or by “blind” techniques, such as, for example, light reflectance, impedance, suction, ultrasound, passive induced microchip, or the magnetic locator described in connection with FIGS. 34A, 34B, 35A, and 35B below.

FIGS. 34A, 34B, 35A, and 35B illustrate a magnetic locating system for enabling “blind” retrieval without the use of an opthalmoscope. To remove the attenuation device from the eye, the removal system is inserted into the eye for intraocular capture, deflation, and extraction of the attenuation device. The removal system utilizes a magnet whose polarity and flux path is oriented in a manner to ensure predictable attraction and coupling of a magnet-containing attenuation device to the removal system. The removal system is coupled back to the attenuation device, and the attenuation device may be punctured and deflated using the jaws of biopsy-like forceps (or other solution suitable for deconstructing the device) located at the distal end of the removal system. In one embodiment, residual gas may be passively vented into the eye or through the retriever body. Once deflated the attenuation device may be withdrawn from the eye attached to the removal system.

Thus, referring to FIG. 34A, there is illustrated an attenuation device 230 such as an inflatable balloon 229 as has been described previously herein. The attenuation device 230 is provided with a valve 232 and a locating element 234. Locating element 234 may be any of the variety of structures which enable location of the attenuation device 230, preferably without the need for direct visualization.

In the illustrated embodiment, the locating element 234 is one or more magnets 236. In the embodiment illustrated in FIG. 34B, the magnet 236 comprises an annular ring, for surrounding the flow path. A corresponding magnet 238 having reversed polarities from the polarity of the magnet 236 is provided on the distal end of a catheter 240. The attractive forces of the opposing polarity magnets 236 and 238 will cause the catheter 240 to couple on to the attenuation device 230, as illustrated in FIG. 34A, when the catheter 240 is positioned in the vicinity of the attenuation device 230.

Referring to FIG. 35A, at least one lumen 242 places the attenuation device 230 in fluid communication with the catheter 240 when the locating element 234 is coupled to the catheter 240. This lumen 242 may be utilized to either introduce inflation media or remove inflation media from the attenuation device 230. In FIG. 35A, the valve 232 is a ball valve, which is biased in the closed orientation. However, the mechanism and structures disclosed herein may be used on any of the other valves disclosed elsewhere herein. In one embodiment, illustrated in FIG. 35A, a valve actuator 233 may be advanced distally through the lumen 242 to displace the valve 232 and enable infusion or removal of inflation media. Following the desired volume of infusion or removal of inflation media, the valve actuator 233 may be proximally retracted, to enable the valve to close under its own bias. See FIG. 35B.

The opposing magnets 236 and 238 may be utilized solely as a locating structure, such that an additional locking element (not illustrated) may be utilized to lock the catheter 240 on to the attenuation device 230. This may be desirable if the strength of the bond formed between the two magnets is insufficient to keep the attenuation device 230 coupled to the catheter 240 during the filling or removal steps. In addition, following deflation of the attenuation device 230, the catheter 240 will generally require a relatively strong coupling to the attenuation device 230 to retrieve the attenuation device 230, as will be apparent to those of skill in the art in view of the disclosure herein.

In another embodiment, the removal system is provided with one or more ultrasound transducers near a distal end thereof. An air filled attenuation device should strongly reflect an ultrasound signal, in a manner similar to the reflection achieved at an air-water interface. A removal system provided with a deflectable distal tip and ultrasonic capabilities should be able to navigate through the eye to locate an attenuation device without the need for visualization. The removal system may additionally be provided with a grasping element, such as two or more opposing mechanical graspers, and/or a vacuum lumen, for attaching to the surface of the attenuation device using suction. Once attached, the attenuation device can be pierced and trans-sclerally withdrawn.

In another embodiment, the delivery system and the removal system of the attenuation device or accumulator are two separate instruments. In another embodiment, the delivery system and the removal system are implemented using a single instrument. In yet another embodiment, there is provided one instrument having different distal ends for the delivery system and the removal system.

In another embodiment, there is provided an implantable self-inflating pressure attenuation device that can inflate from a first, deflated configuration to a second, at least partially inflated configuration. Various transformable mediums can be used to inflate the housing of the attenuation device from a deflated configuration to at least a partially inflated configuration.

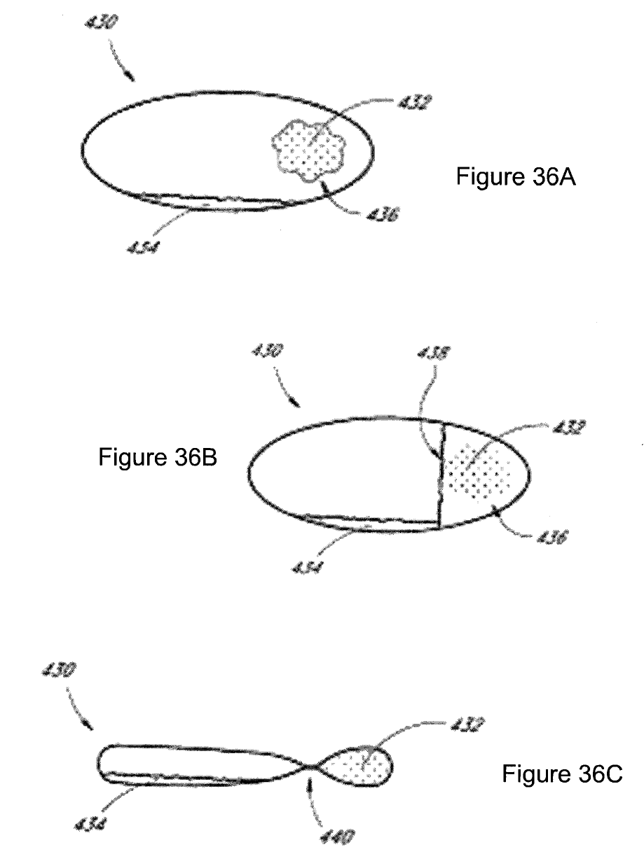

With reference to FIGS. 36A-36C, in one embodiment, the transformable medium comprises a first reactant 432 and a second reactant 434. Here, the implantable self-inflating pressure attenuation device 430 (shown in its first, deflated configuration) generally comprises a first reactant 432 and a second reactant 434, which are physically separated from each other. When the first reactant 432 comes into contact the second reactant 434, a chemical reaction occurs within the attenuation device 430, thereby causing the device attenuation 430 to transform into at least a partially inflated configuration (not illustrated).

With reference to FIG. 36A, in one embodiment, the first reactant 432 is contained within a balloon or container 436 that is entirely contained within and free to move within the attenuation device 430. The container 436 is generally impermeable to reactants 432, 434, and can comprise any suitable material known to those skilled in the art. The suitability of a material for the container 436 will depend on the chemical characteristics of the reactants 432, 434. In another embodiment, illustrated in FIG. 36B, the reactants 432, 434 are compartmentalized and separated within the attenuation device 430 by a wall 438. The wall 438 is generally impermeable to reactants 432, 434, and can comprise any suitable material known to those skilled in the art. The suitability of a material for the wall 438 will depend on the chemical characteristics of the reactants 432, 434. In yet another embodiment, shown in FIG. 36C, the attenuation device 430 has a crease 440. The crease 440 separates the reactants 432, 434, and thereby prevents the inflation/expansion reaction from occurring until such inflation/expansion is desired and triggered by the user. In still another embodiment (not illustrated), the reactants 432, 434 are separated within the attenuation device 430 by a peelable bond, fold, and/or the like, known to those skilled in the art.

In one embodiment, the medium capable of transformation comprises gas generating compositions. Various compositions can be used to generate gas. One class of compositions is the combination of a base and an acid to produce carbon dioxide. The acid and base are combined in dry form and rendered reactive only when co-dissolved in water. Examples of suitable bases are water-soluble carbonate and bicarbonate salts, non-limiting examples of which are sodium bicarbonate, heat treated sodium bicarbonate, sodium carbonate, magnesium carbonate, potassium carbonate, and ammonium carbonate. Non-limiting examples of suitable acids are citric acid, tartaric acid, acetic acid, and fumaric acid. One presently preferred composition is a dry mixture of sodium bicarbonate and citric acid. Compositions containing more than one acid component or base component can also be used.

Gas generation can be initiated various ways, such as, for example, contact with a fluid, temperature change, ignition, pH change, etc. In one embodiment, the amount of gas generated is equal to the amount of volume dissipated through the air cell, thereby allowing for constant volume device until the gas generating materials are exhausted.

The amount and rate of gas production can be controlled by certain factors, such as, for example, the amount of reactive materials or reactants, the amount of gas entrapped in the structure, or the solubility of one or both of the chemicals in water, etc. In one embodiment comprising a wick and tablet systems, the available water as delivered by the wick to the tablet dissolves only a limited amount of the reactants and resulting reaction product(s). The reaction is thus limited by the solubility of the chemicals in the limited amount of available water. The rate of water delivery thereby controls the reaction rate. Some examples of the solubility of suitable reaction chemicals per 100 grams of water are as follows: sodium bicarbonate, about 10 g; citric acid, about 200 g; tartaric acid, about 20 g; and fumaric acid, about 0.7 g. The limited solubility and limited water delivery rate through the wick make it unnecessary to keep the acid and base separated either before or during use of the infusion device.

It is further understood that a catalyst, another chemical species or one of the byproducts of the reaction can propagate the reaction and increase its speed. In the case of sodium bicarbonate and citric acid, the byproducts are carbon dioxide, sodium citrate, and water. A very small amount of water, such as, for example, 0.01 to 0.5 ml, can be used to start the reaction by dissolving the sodium carbonate and citric acid. Since water is produced in the reaction, the reaction speed increases until all of the reactants are exhausted.

As a manufacturing aid, it may be desirable to add inert agent(s) to the reactant composition to aid in the tableting process and to keep the tablet intact during and after use. Examples of suitable tableting aids include but are not limited to polyvinyl pyrrolidone and anhydrous dibasic calcium phosphate, sold by Edward Medell Co. (Patterson, N.J., USA) as EMCOMPRESS.RTM. Tableting aids can be eliminated for certain compositions with no loss of performance. One such composition is the mixture of sodium bicarbonate and citric acid.

Chemical compositions that produce oxygen or other gases can also be used. A composition to generate oxygen in the presence of water is disclosed in U.S. Pat. No. 4,405,486, titled Method for Preparing granulated perborate salts containing a polymeric fluorocarbon, issued Sep. 20, 1983, the disclosure of which is incorporated in its entirety herein by reference. The controlled rate of wicking water into such a tablet, and the limited solubility of the constituents can control the rate of oxygen release in a manner similar to that of carbon dioxide in the systems described above.

In another embodiment, the medium capable of transformation comprises peroxide and/or superoxide chemical systems. In certain embodiments, gas is generated by drawing an aqueous solution of a peroxide or superoxide into an absorbent tablet that contains an enzyme or catalyst which promotes the decomposition of the peroxide or superoxide to decomposition products including oxygen gas. In another embodiment, a solid peroxide or superoxide can be incorporated into the tablet, with oxygen generation being initiated by contact of the peroxide or superoxide with water. Hydrogen peroxide, for example, decomposes into water and oxygen, providing no hazardous reaction products after infusion of the liquid has been completed. Metal peroxides, such as, for example, lithium peroxide, sodium peroxide, magnesium peroxide, calcium peroxide, and zinc peroxide, etc., react with water to produce the metal hydroxide and hydrogen peroxide, which then decomposes into water and oxygen. Superoxides, such as, for example, sodium superoxide, potassium superoxide, rubidium superoxide, cesium superoxide, calcium superoxide, tetramethylammonium superoxide, etc., react with water to produce the metal hydroxide and oxygen gas directly. It will be noted that the production of hydrogen peroxide itself is particularly preferred.

In one embodiment, a suitable tablet contains a water absorbent material to facilitate the wicking action, and the enzyme or catalyst in systems where enzymes or catalysts are used. Examples of water absorbents useful for this purpose include superabsorbent polymers, reconstituted cellulosic materials, compressed zeolite powder (Types 13X and 4A, both unactivated), etc.

One example of a suitable enzyme is catalase. Lyophilized catalases are generally preferred. Catalysts effective for the decomposition include metals deposited on high surface area substrates, such as, for example, alumina, activated carbon, etc. Examples of suitable catalysts include platinum, palladium, silver, etc.

Chemical reactants can also be used rather than enzymes or catalysts to decompose hydrogen peroxide. Examples of such reactants include but are not limited to potassium permanganate, sodium hydroxide, etc. It should be noted, however, that there are safety concerns associated with potassium permanganate and sodium hydroxide.

As between enzymes and catalysts, enzymes provide a cost benefit for single-use systems. For reusable systems, however, catalysts are generally preferred. One significant advantage to the use of a hydrogen peroxide system with a catalyst is the ability to regenerate the system by drying out the tablet and adding more hydrogen peroxide solution to the water reservoir. Regeneration in this type of system is thus easier than regeneration of an absorbent tablet for a system that requires adsorbed gas.

In another embodiment, the medium capable of transformation comprises chemical reactants that are used effectively to generate a gas to push a fluid from an infusion pump. In order to generate carbon dioxide, two or more reactive chemicals are mixed that, upon reaction, generate a gas. Preferably, one of the reactants is provided in liquid form, i.e., a liquid chemical, a solution, or the like, and another one of the reactants is provided as a solid. Either the liquid or the solid may comprise more than one reactive chemical. However, in one preferred embodiment, each of the liquid and the solid contain only one reactive species.

Carbon dioxide is generally quite inert and safe at low concentrations. However, other gases could also be used, provided they are relatively inert and safe. For the purposes of the following discussion, it will be assumed that carbon dioxide is to be generated. As mentioned above, to generate the gas, at least two reactants are caused to come into contact. For ease of reference, the reactants will be referred to herein as a first reactant and a second reactant or a solid reactant and a liquid reactant, and particular sets of reactants will be referred to as reactant sets.

First Reactant: Preferably, the first reactant is selected from a group consisting of carbonates and bicarbonates, particularly, Group I and II metal carbonates and bicarbonates (the “carbonate”). For example, in one embodiment, preferred carbonates include sodium bicarbonate, sodium carbonate, magnesium carbonate, and calcium carbonate. However, sodium bicarbonate, sodium carbonate and calcium carbonate are highly preferred, with sodium carbonate (or soda ash) being the most highly preferred. One desirable feature of sodium carbonate is that it is easily sterilizable. For example, sodium carbonate can be sterilized with heat, such as through autoclaving. This is preferable, since the infusion devices are designed for human use and it is safer to ensure that all of the components are sterile whether it is expected that they will come into contact with the patient or not. Other reactants that are sterilizable with heat, ethylene exposure, or exposure to ionizing radiation are equally useful.

The carbonate can be either used as a solid reactant or can be dissolved in a solution to form a liquid reactant. In one preferred embodiment, the carbonate is used as a solid. The reason for this choice is that the carbonates are all solids and some are only sparingly soluble in water.

Second Reactant: The second reactant is preferably an acid. Preferably, the acid is selected from the group consisting of acids, acid anhydrides, and acid salts. Preferably, the second reactive chemical is citric acid, acetic acid, acetic anhydride, or sodium bisulfate. Usually the second reactant is used as the liquid reactant. However, in the case of citric acid and sodium bisulfate, for example, the second reactant can also be the solid reactant. Nevertheless, the second reactant is generally more soluble in water than the first reactant and is, therefore, used to form the liquid reactant.

Reactant Sets: A reactant set is based upon a variety of considerations. For example, the solubilities of the first and second reactants are considered to determine which reactant should be used as the solid or liquid reactant. Also considered is the product of the reaction and its solubility. It is preferred that the products be CO₂ gas and a soluble inert compound. Once these factors are considered, appropriate reactant sets can be constructed. For instance, in one embodiment, reaction sets such as those shown in Table I are preferred.

TABLE I

| Solid Reactant | Liquid Reactant |
|---|---|
| Sodium Carbonate | Citric Acid |
| Calcium Carbonate | Acetic Acid |
| Magnesium Carbonate | Citric Acid |

Additional details may be found in U.S. Pat. No. 5,992,700, titled controlled gas generation for gas-driven infusion devices, issued Nov. 30, 1999, and U.S. Pat. No. 5,588,556, titled method for generating gas to deliver liquid from a container, issued Dec. 31, 1996. Both of these patents are hereby incorporated by reference herein and made a part of this specification.

In another embodiment, the method of producing gas is entrapped pressurized gas in a sugar or a porous molecular sieve. Generally, gas is liberated when the structure comes in contact with a fluid.

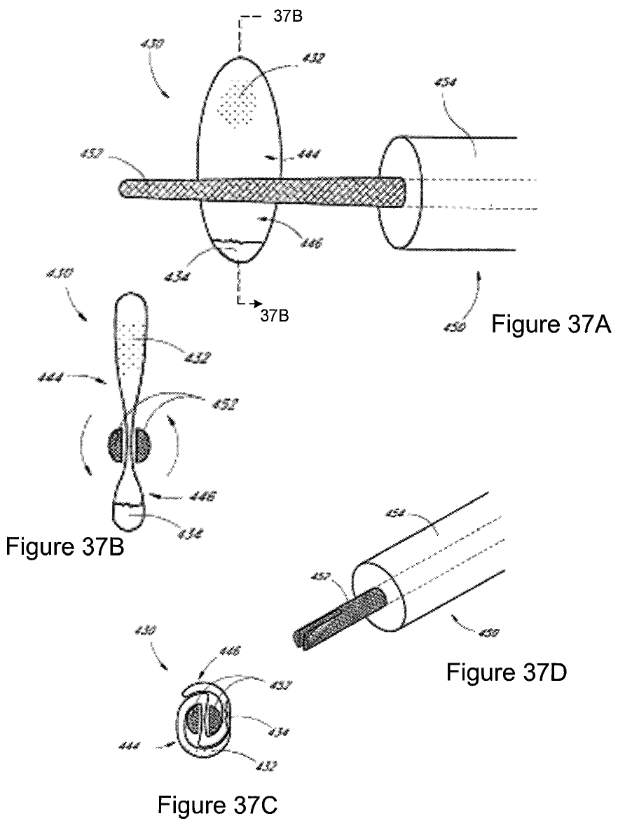

In another embodiment, there is provided a method of delivering the implantable self-inflating pressure attenuation device 430 into the treatment site, such as, for example, the eye. With reference to FIGS. 37A-37D, in one embodiment, the delivery system 450 includes a bifurcated delivery tool 452 and a delivery cannula 454. The tool 452 has a fork-like shape and can be extended out and retracted into the cannula 454. As illustrated, the bifurcations of the tool 452 are spaced so as to squeeze or pinch the device 430, thereby separating a first portion 444 of the attenuation device 430 from a second portion 446, and thereby separating a first reactant 432 from a second reactant 434. Because the reactants 432, 434 do not come into contact with each other, the device remains in its deflated state, thereby facilitating the procedure of delivering the attenuation device 430 to the treatment site, such as, for example, the eye. In one embodiment, shown in FIGS. 37B and 37C, first and second portions 444, 446 of the deflated attenuation device are wound about itself along the axis of the tool 452, thereby minimizing the volume of the attenuation device 430, and thereby facilitating the delivery of the attenuation device 430 into the treatment site.

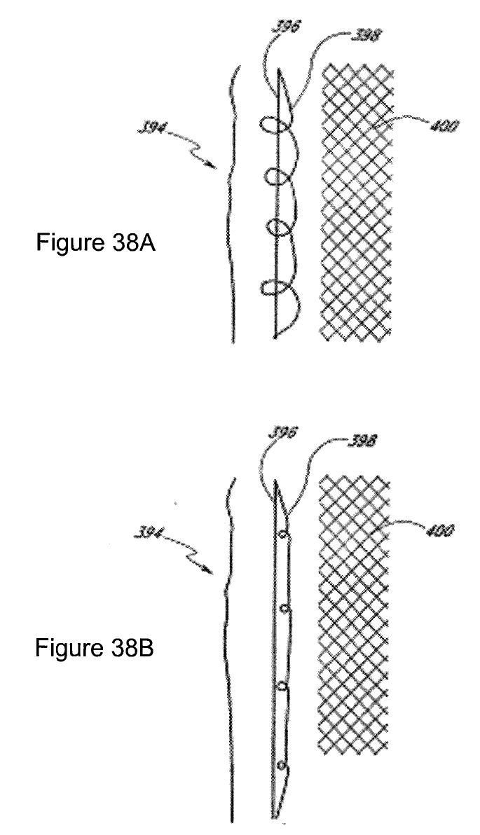

The tissue of the eye is composed of transitional epithelium. Beneath it is a well-developed layer formed largely of connective and elastic tissues. With reference to FIGS. 38A and 38B, the connective and elastic tissues of the eye wall generally comprise-elastin 396, collagen 398, and extracellular matrix 400.

With reference to FIG. 38A, as in most tissues, collagen 398 is arranged as a coiled or complex helical material within the eye wall. While collagen 398 itself is not very elastic (distensible), the coiled configuration allows expansion of the collagen bundle. When the bundle is extended (see FIG. 38B), the uncoiled collagen length becomes the limiting size. It is at this point that tension rises rapidly, analogous to the twisting of several strands of rope. When twisted, the combined strands shorten. The combined strands can be lengthened by untwisting without stretching any individual strand. As in other tissues, as the patient ages, the extensibility of the elastin 396 and the collagen 398 reduces due to age-induced crosslinking, reducing the compliance of the eye 63.

In the cardiovascular, pulmonary, and urology fields, it has been found that the removal of excessive mechanical stress and strain on various tissues for a period of time allows the cells and the tissue to recover and improve function. In the eye, reducing the stress induced by high frequency, repetitive pressure spikes increases the dynamic compliance of the eye and reduces symptoms of high intraocular pressure by: allowing the “stretched” muscles of the eye wall to shorten, thereby improving compliance and eye wall contractility. By removing pressures exerted on the connective tissues, allowing retraining and healing; placing the attenuation device in the eye provides passive resistance to the wall, allowing the muscles to strengthen. These and other therapeutic benefits could last up to about 30 days to about one year. One additional benefit of attenuation and/or improving eye compliance includes improved flow of aqueous humor (i.e. method of improving flow by “smoothing” the pressure within the eye).

In another embodiment of the device, the attenuator is used to treat progressive myopia. Progressive myopia may result from an inherited biomechanical weakness of the sclera that allows it to stretch in response to stress. Increased intraocular pressure could be the mediator of stress produced by the inclined head position and the accommodation/convergence aspects of near work. A pressure attenuator like any of those described herein could be used to attenuate the pressure waves that lead to stretching of the sclera and other vision-producing structure. In so doing, the attenuator device would treat ocular disorders related to refractive error.

In another embodiment described herein, the attenuation device may also be used to treat presbyopia, by unloading the mechanical stress on the ciliary muscles which control accommodation of the crystalline lens. By reducing/removing the stress on the ciliary muscles, the tissue can regain more of its original compliance, restoring accommodative amplitude to the eye.

In another embodiment described herein, the attenuation device can provide temporary protection to the optic nerve and other ocular tissues during traumatic ocular procedures that would result in temporary or transient pressure spikes to the eye, such as muscular-orbital procedures, scleral surgeries, or procedures involving applanation, such as during LASIK. In such instances, the attenuation device may remain in the eye during the procedure itself, during the postoperative period, or for some period of time thereafter, as long as is required to provide protection to the eye.

In another embodiment described herein, the attenuation device may also deliver drug or drug combinations locally. It may be coated with various drugs and/or polymer/drug combinations which have a therapeutic effect, or aid in the tolerability or performance of the device. Various biocompatible, non-biodegradable polymeric compositions may be employed in the implants. The non-biodegradable polymeric composition employed must allow for release of the drug by, for example, solution/diffusion or leaching mechanisms. The non-biodegradable polymeric compositions employed may be varied according to the compatibility of the polymer with the drug or other active agent to be employed, ease of manufacture, the desired rate of release of the drug, desired density or porosity, and the like. Various non-biodegradable polymers which may be employed are described in U.S. Pat. Nos. 4,303,637; 4,304,765; 4,190,642; 4,186,184; 4,057,619; 4,052,505; 4,281,654; 4,959,217; 4,014,335; 4,668,506; 4,144,317. The non-biodegradable polymers may be homopolymers, copolymers, straight, branched-chain, or cross-linked derivatives.

Exemplary biocompatible, non-biodegradable polymers of particular interest include polycarbonates or polyureas, particularly polyurethanes, polymers which may be crosslinked to produce non-biodegradable polymers such as cross-linked poly(vinyl acetate) and the like. Also of particular interest are ethylene-vinyl ester copolymers having an ester content of 4 to 80% such as ethylene-vinyl acetate (EVA) copolymer, ethylene-vinyl hexanoate copolymer, ethylene-vinyl propionate copolymer, ethylene-vinyl butyrate copolymer, ethylene-vinyl pentantoate copolymer, ethylene-vinyl trimethyl acetate copolymer, ethylene-vinyl diethyl acetate copolymer, ethylene-vinyl 3-methyl butanoate copolymer, ethylene-vinyl 3-3-dimethyl butanoate copolymer, and ethylene-vinyl benzoate copolymer. Ethylene-vinyl ester copolymers including ethylene-vinyl acetate copolymers for the manufacture of diffusional ocular drug delivery devices where the drug dissolves in and passes through the polymer by diffusion are described in U.S. Pat. Nos. 4,052,505 and 4,144,317.

Additional exemplary naturally occurring or synthetic non-biodegradable polymeric materials include poly(methylmethacrylate), poly(butylmethacrylate), plasticized poly(vinylchloride), plasticized poly(amides), plasticized nylon, plasticized soft nylon, plasticized poly(ethylene terephthalate), natural rubber, silicone, poly(isoprene), poly(isobutylene), poly(butadiene), poly(ethylene), poly(tetrafluoroethylene), poly(vinylidene chloride), poly(acrylonitrile, cross-linked poly(vinylpyrrolidone), poly(trifluorochloroethylene), chlorinated poly(ethylene), poly(4,4′-isopropylidene diphenylene carbonate), vinylidene chloride-acrylonitrile copolymer, vinyl chloridediethyl fumarate copolymer, silicone, silicone rubbers (especially the medical grade), poly(dimethylsiloxanes), ethylene-propylene rubber, silicone-carbonate copolymers, vinylidene chloride-vinyl chloride copolymer, vinyl chloride-acrylonitrile copolymer, vinylidene chloride-acrylonitrile copolymer, poly(olefins), poly(viny-olefins), poly(styrene), poly(halo-olefins), poly(vinyls), poly(acrylate), poly(methacrylate), poly(oxides), poly(esters), poly(amides), and poly(carbonates).

Biodegradable or non-biodegradable hydrogels may also be employed in the implants disclosed herein. Hydrogels are typically a copolymer material, characterized by the ability to imbibe a liquid. Exemplary non-biodegradable hydrogels which may be employed and methods of making these hydrogels are described in U.S. Pat. Nos. 4,959,217 and 4,668,506, herein incorporated by reference. Exemplary biodegradable hydrogels which may be employed are described in U.S. Pat. No. 4,957,998.

Where a non-biodegradable polymer is employed, the rate of release of the drug will be primarily solution/diffusion controlled. The rate of diffusion of drug through the non-biodegradable polymer may be affected by drug solubility, polymer hydrophilicity, extent of polymer cross-linking, expansion of the polymer upon water absorption so as to make the polymer more permeable to the drug, and the like. Diffusion of the drug from the implant may also be controlled by the structure of the implant. For example, diffusion of the drug from the implant may be controlled by means of a membrane affixed to the polymer layer comprising the drug. The membrane layer will be positioned intermediate to the polymer layer comprising the drug and the desired site of therapy. The membrane may be composed of any of the biocompatible materials indicated above and may vary with the drug employed, the presence of agents in addition to the drug present in the polymer, the composition of the polymer comprising the drug, the desired rate of diffusion and the like. For example, the polymer layer will usually comprise a very large amount of drug and will typically be saturated. Such drug-saturated polymers may generally release the drug at a very high rate. In this situation, the release of the drug may be slowed by selecting a membrane which is of a lower drug permeability than the polymer. Due to the lower drug permeability of the membrane, the drug will remain concentrated in the polymer and the overall rate of diffusion will be determined by the drug permeability of the membrane. Therefore, the rate of release of the drug from the implant is reduced, providing for a more controlled and extended delivery of the drug to the site of therapy.

Where the implant comprises a polymer layer comprising the drug and/or a membrane layer, it may be desirable for the implant to further comprise a backing layer. The backing layer will be in contact with the surfaces of the implant which are not in contact with or adjacent the desired site of therapy. For example, where the implant is a sheet, the backing layer may be present on the side of the sheet which is to be most distant from the desired site of therapy. In this instance, the backing layer may not be necessary on the edges of the sheet as the surface area of this portion of the implant is fairly insignificant and one would therefore expect loss of the drug from the polymer at this site to be minimal. The composition of the backing may vary with the drug employed in the implant, the site of implantation, compatibility with agents in addition to the drug which may be employed in the implant and the like. Of particular importance is that the backing be composed of a biocompatible, preferably non-biodegradable, material which is impermeable to the drug contained within the polymer layer. Thus diffusion of the drug from the polymer layer will only be allowed by passage through the polymer and/or membrane layer and any intervening ocular membranes to the desired site of treatment. Exemplary compositions for the backing include polyesters (e.g., mylar), polyethylene, polypropylene, teflon, aclar and other film material which are well known and/or commercially available.

The implant may further comprise an adhesive layer for securing the implant at the desired insertion site, particularly where the implant is to be placed substantially on the outer surface of the eye over an avascular region. Preferably, the adhesive layer will be on the portion of the implant in direct contact with the ocular membrane and over the desired site of treatment. Where desired, the polymer layer may be affixed to a release liner or peel strip. The release liner, which may be of any suitable material which is impermeable to the drug, will serve to prevent diffusion of the drug out of the polymer during storage. Where the implant comprises an adhesive coating, the release liner will prevent the adhesive layer from adhering to packing material, other implants, and the like. Typically the release liner will be a polyester layer coated with a release agent such as a silicone or fluorocarbon agent to facilitate removal of the release liner from the polymer prior to insertion of the implant into the eye.

For the most part, the non-biodegradable implants will have indefinite lifetimes within the eye and may be removed when either release of the drug from the polymer is complete or when therapy is no longer needed or efficacious. The period of drug administration may be varied by the amount of drug contained within the polymeric implant, the size or shape of the implant, and the like. Implant comprising non-biodegradable polymers will usually provide for diffusion of the drug for at least 2 weeks more usually at least 4 weeks, generally at least about 12 weeks and may be 24 weeks or more. The implants may be removed when therapy is completed or no longer efficacious.

Where, for example, the molecular weight of the drug, the desired dosage, the period of administration (as in chronic therapy) and the like are such that the size of the implants required to contain the desired amount of drug or drug solution is incompatible with the size of the insertion site or would compromise the patient's vision, employment of a non-biodegradable implant comprising a refillable reservoir may be desired. Non-biodegradable, refillable reservoirs may comprise a non-biodegradable outer surface and a hollow or substantially hollow center which acts as the depot, or reservoir, for the active agent. The active agent may be present in a variety of forms including initially dry; in a suspension comprising a physiological buffer such as saline, a permeability enhancing agent such as ethanol, or a preservative such as EDTA; in a suspension comprising a biodegradable polymeric composition; in a suspension comprising a biodegradable gel, or the like. The implant may be refilled with any one or all of the components present in the original active agent suspension contained within the implant. The implant may be placed into the desired site of insertion, so that it will not substantially migrate from the site of insertion.

The implant may be refilled by, for example, injection of the active agent directly into the reservoir of the implant. It is of particular importance to the operability of implants comprising refillable reservoirs that refilling of the implant does not compromise the ability of the implant to release the active agent at the desired rate. Therefore, it is preferable that the outer surface of the implant will comprise a self-sealing layer. The self-sealing layer may be comprised of a non-biodegradable material and may be a rubber-like material or other material which is capable of resealing. Injection of the active agent or active agent suspension through the self-sealing layer will not result in the production of a hole at the site of injection. Alternatively, the refillable implant may comprise an inlet. The inlet may comprise a hollow fiber which may be positioned so as to communicate with the outer surface of the implant and with the reservoir within the body of the implant. The portion of the inlet which communicates with the outer surface of the implant will be of a self-sealing composition or will be capable of being resealed or otherwise treated so as to prevent loss of the drug from the reservoir through the inlet. Implants with such inlets may be refilled by injection of the active agent through the hollow fiber. In addition, where the implant is placed within the tissue layers of the eye (e.g., between the scleral layers), the inlet of the implant may be positioned so as to be accessible from the outer surface of the eye for refilling of the implant reservoir.

Following insertion, the refillable, non-biodegradable implant will provide for diffusion of the drug contained therein for at least 2 weeks, more usually at least 4 weeks, generally at least about 8 weeks and may be 6 months or more. After diffusion of the drug is complete, the reservoir may be refilled by means of injection of the drug or drug suspension into the implant. Alternatively, the implant may comprise an inlet which communicates with the outer surface of the implant and with the internal reservoir. The drug or drug suspension may then be injected through the inlet to refill the implant. An example of an implant comprising a refillable reservoir is described in U.S. Pat. No. 4,300,557. The refillable implants may be employed in the eye of the patient for the entire course of therapy and may be employed for at least 2 weeks, more usually at least 4 weeks, generally at least about 8 weeks and may be 6 months or more. The implants may be removed when therapy is completed or no longer efficacious.

Biodegradable polymeric compositions which may be employed may be organic esters or ethers, which when degraded result in physiologically acceptable degradation products, including the monomers. Anhydrides, amides, orthoesters or the like, by themselves or in combination with other monomers, may find use. The polymers may be addition or condensation polymers, particularly condensation polymers. The polymers may be cross linked or non-cross-linked, usually not more than lightly cross-linked, generally less than 5%, usually less than 1%. For the most part, besides carbon and hydrogen, the polymers will include oxygen and nitrogen, particularly oxygen. The oxygen may be present as oxy, e.g., hydroxy or ether, carbonyl, e.g., non-oxo-carbonyl, such as carboxylic acid ester, and the like. The nitrogen may be present as amide, cyano and amino. The polymers set forth in U.S. Pat. No. 5,013,821 may find use, and that disclosure is specifically incorporated herein by reference.

Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate, a slowly eroding polymer is achieved, while erosion is substantially enhanced with the lactate racemate.

Among the polysaccharides will be calcium alginate, and functionalized celluloses, particularly carboxymethylcellulose esters characterized by being water insoluble, a molecular weight of about 5 kD to 500 kD, etc. Other polymers of interest include polyvinyl alcohol, esters and ethers, which are biocompatible and may be biodegradable or soluble. For the most part, characteristics of the polymers will include biocompatibility, compatibility with the agent of interest, ease of encapsulation, a half-life in the physiological environment of at least 6 hrs; preferably greater than one day, no significant enhancement of the viscosity of the vitreous, water insoluble, and the like.

The biodegradable polymers which form the implants will desirably be subject to enzymatic or hydrolytic instability. Water soluble polymers may be crosslinked with hydrolytic or biodegradable unstable cross-links to provide useful water insoluble polymers. The degree of stability can be varied widely, depending upon the choice of monomer, whether a homopolymer or copolymer or mixtures of polymers are employed, where the polymers may be employed as varying layers or mixed.

By employing a biodegradable polymer, particularly one where the biodegradation is relatively slow, the rate of release of the drug will be primarily diffusion controlled, depending upon the nature of the surrounding membrane or monolithic polymer structure, rather than polymer degradation leading to disintegration of the implant. For the most part, the selected particles will have lifetimes at least equal to the desired period of administration, preferably at least twice the desired period of administration, and may have lifetimes of 5 to 10 times the desired period of administration. The period of administration will usually be at least 3 days, more usually at least 7 days, generally at least about 15 days and may be 20 days or more.

The particles used to form the devices for implantation may be substantially homogeneous as to composition and physical characteristics or heterogeneous. Thus, particles can be prepared where the center may be of one material and the surface may have one or more layers of the same or different composition, where the layers may be cross-linked, of different molecular weight, different density or porosity, or the like. For example, the center could be a polylactate coated with a polylactate-polyglycolate copolymer, so as to enhance the rate of initial degradation. Most ratios of lactate to glycolate employed will be in the range of about 1:0.1. Alternatively, the center could be polyvinyl alcohol coated with polylactate, so that on degradation of the polylactate the center would dissolve and be rapidly washed out of the eye. Implants may also be composed of biodegradable and non-biodegradable polymers. For example, the implant may comprise an outer surface made of a non-biodegradable polymeric material surrounding an inner core of biodegradable material. The rate of release of the active agent would then be influenced by both the release of the agent from the biodegradable center and subsequent diffusion of the drug through the outer non-biodegradable layer.

Any pharmacologically active agent for which sustained release is desirable may be employed including drugs, pharmaceutical agents, bacterial agents, etc. The agents will be capable of diffusion into the vitreous to be present at an effective dose. In this manner, drugs or pharmaceutical agents will be sufficiently soluble to be presented at pharmacologically effective doses. Pharmacologic agents which may find use may be found in U.S. Pat. Nos. 4,474,451, columns 4-6, and 4,327,725, columns 7-8, which disclosures are incorporated herein by reference.

Bacterial agents include acid fast bacilli, (BCG), Corynebacterium parvum, LPS, endotoxin etc. These agents induce an immune response enhancing immune attack of tumor cells. These agents are frequently used as immune adjuvants to enhance an immune response to an administered antigen. See Morton et al., Surgery (1970) 68:158-164; Nathanson, L., Cancer Chemother. Rep. (1973) 56:659-666; Pinsky et. al., Proc. AACR (1972) 13:21; and, Zhar et. al., J. Nat'l Cancer Inst. (1971) 46:831-839.

Drugs of particular interest include hydrocortisone, gentamicin, 5-fluorouracil, sorbinil, IL-2, TNF, Phakan-a (a component of glutathione), thiolathiopronin, Bendazac, acetylsalicylic acid, trifluorothymidine, interferon (α, β and γ), immune modulators, e.g., lymphokines, monokines, and growth factors, cytokines, anti-(growth factors), etc.

Other drugs of interest include drugs for treatment of macular degeneration, such as interferon, particularly α-interferon; transforming growth factor (TGF), particularly TGF-β; insluin-like growth factos; anti-glaucoma drugs, such as the beta-blockers: timolol maleate, betaxolol and metipranolol; mitotics: pilocarpine, acetylcholine chloride, isofluorophate, demecarium bromide, echothiophate iodide, phospholine iodide, carbachol, and physostigmine; epinephrine and salts, such as dipivefrin hydrochloride; and dichlorphenamide, acetazolamide and methazolamide; anti-cataract and -diabetic retinopathy drugs, such as aldose reductase inhibitors: tolrestat, lisinopril, enalapril, and statil; thiol cross-linking drugs other than those considered previously; anti-cancer drugs, such as retinoic acid, methotrexate, adriamycin, bleomycin, triamcinolone, mitomycin, cis-platinum, vincristine, vinblastine, actinomycin-D, ara-c, bisantrene, CCNU, activated cytoxan, DTIC, HMM, melphalan, mithramycin, procarbazine, VM26, VP16, and tamoxifen; immune modulators, other than those indicated previously; anti-clotting agents, such as tissue plasminogen activator, urokinase, and streptokinase; anti-tissue damage agents, such as superoxide dismutase; proteins and nucleic acids, such as mono and polyclonal antibodies, enzymes, protein hormones and genes, gene fragments and plasmids; steroids, particularly anti-inflammatory or anti-fibrous drugs, such as cortisone, hydrocortisone, prednisolone, prednisone, dexamethasone, progesterone-like compounds, medrysone (HMS) and fluorometholone; non-steroidal anti-inflammatory drugs, such as ketrolac tromethamine, diclofenac sodium and suprofen; antibiotics, such as loridine (cephaloridine), chloramphenicol, clindamycin, amikacin, tobramycin, methicillin, lincomycin, oxycillin, penicillin, amphotericin B, polymyxin B, cephalosporin family, ampicillin, bacitracin, carbenicillin, cephalothin, colistin, erythromycin, streptomycin, neomycin, sulfacetamide, vancomycin, silver nitrate, sulfisoxazole diolamine, quinolones, and tetracycline; other anti-pathogens, including anti-fungal or anti-viral agents, such as idoxuridine, trifluorouridine, vidarabine (adenine arabinoside), acyclovir (acycloguanosine), gancyclovir, pyrimethamine, trisulfapyrimidine-2, clindamycin, nystatin, flucytosine, natamycin, miconazole, ketoconazole, aromatic diamidines (e.g., dihydroxystilbamidine) and piperazine derivatives, e.g. diethylcarbamazine; cycloplegic and mydriatic agents, such as atropine, cyclogel, scopolamine, homatropine and mydriacyl.

Other agents include anticholinergics, anticoagulants, antifibrinolytic agents, antihistamines, antimalarials, antitoxins, chelating agents, hormones, immunosuppressives, thrombolytic agents, vitamins, salts, desensitizing agents, prostaglandins, amino acids, metabolites and antiallergenics.

The amount of agent employed in the implant will vary widely depending on the effective dosage required and rate of release. Usually the agent will be from about 1 to 80, more usually 20 to 40 weight percent of the implant.

Other agents may be employed in the formulation for a variety of purposes. For example, agents which increase drug solubility, buffering agents and preservatives may be employed. Where the implant is positioned such that no portion of the implant is in direct contact with the vitreous, diffusion of the drug into the eye (for example across the conjunctiva, sclera and choroid to reach the vitreous) may be facilitated by enhancers (i.e. DMSO, detergents, ethanol, isopropyl myristate (IPM), oleic acid, azome and the like). Enhancers may act either to increase the permeability of ocular membranes through which the active agent must diffuse in order to reach the desired site within the eye or may serve to increase drug solubility within the vitreous. The enhancer employed will vary with the drug, as well as the polymer, employed in the implant. Water soluble preservatives which may be employed include sodium bisulfite, sodium thiosulfate, ascorbate, benzalkonium chloride, chlorobutanol, thimerosal, phenylmercuric borate, parabens, benzyl alcohol and phenylethanol. These agents may be present in individual amounts of from about 0.001 to about 5% by weight and preferably about 0.01 to about 2%. Suitable water soluble buffering agents which may be employed are alkali or alkaline earth carbonates, phosphates, bicarbonates, citrates, borates, acetates, succinates and the like, such as sodium phosphate, citrate, borate, acetate, bicarbonate and carbonate. These agents may be present in amounts sufficient to maintain a pH of the system of between 2 to 9 and preferably 4 to 8. As such the buffering agent may be as much as 5% on a weight to weight basis of the total composition.

The implants may also be of any geometry desired to incorporate the drug-containing component, including fibers, sheets, films, microspheres, circular discs, plaques and the like. The upper limit for the implant size will be determined by factors such as eye toleration for the implant, size limitations on insertion into the avascular region, ease of handling, etc. Where sheets or films are employed, the sheets or films will be in the range of at least about 0.5 mm×0.5 mm, usually about 3-10 mm×5-10 mm with a thickness of about 0.25-1.0 mm for ease of handling. Where fibers are employed, the diameter of the fiber will generally be in the range of 0.1 to 1 mm. The length of the fiber will generally be in the range of 0.5-5 mm. The size and form of the implant can be used to control the rate of released period of treatment, and drug concentration in the eye. In some situations mixtures of drug-containing components may be utilized employing the same or different pharmacological agents into one attenuation device. In this way, in a single administration a course of drug treatment may be achieved, where the pattern of release may be greatly varied.

Various techniques may be employed to produce the implants. Useful techniques include solvent evaporation methods, phase separation methods, interfacial methods, extrusion methods, molding methods, injection molding methods, heat press methods and the like.

In preparing the polymeric, drug-comprising implants, for the most part solvent-evaporation methods will be employed. Where the implants are to be in the form of microcapsules or microparticles, the preformed rate controlling polymer is dissolved in a volatile substantially water-immiscible solvent, such as chloroform, methylene chloride, or benzene. Sometimes, the water immiscible solvent will be modified with a small amount of a water-miscible organic cosolvent, particularly an oxygenated solvent, such as acetone, methanol, ethanol, etc. Usually, the water-miscible organic cosolvent will be less than about 40 vol % usually less than about 25 vol %. The agent may then be added to the polymer-solvent solution. Depending upon the nature of the agent, one may have the agent dispersed in the viscous polymer-solvent mixture or a solid dispersion of drug particles, where the drug will have been pulverized to obtain a fine powder, usually a microfine powder particularly of a size of less than about 1 mM, usually less than about 0.5 mM, and may be about 0.5 μM or smaller. Where polymeric hydrogels are employed, particularly non-biodegradable polymeric hydrogels, it may be desirable to add a catalyst to achieve polymerization of the drug-solvent solution. Methods for the production of non-biodegradable hydrogels are well-known in the art and are described in U.S. Pat. Nos. 4,668,506 and 4,959,217.

The amount of polymer employed in the medium will vary with the size of the implant desired, whether additional coatings will be added, the viscosity of the solution, the solubility of the polymer and the like. Usually, the concentration of polymer will be in the range of 10 to 80 weight percent. The ratio of agent to polymer will vary with the desired rate of release, the amount of agent generally varying in the range of 1 to 80 weight percent of the polymer in addition to other agents present.

The ratio of drug to polymer may be adjusted to produce optimized compositions, since the final product will normally result in the initial ratio. By manipulating the initial bulk viscosity of the drug-polymer-solvent mixture and of the aqueous dispersing medium, the dissolved polymer agent/mixture may also be added to a rapidly stirred aqueous solution. In this instance the polymer mixture will coalesce in the absence of a dispersing agent, resulting in a large sheet or mass of encapsulation or macroencapsulation. Macroencapsulation can also be achieved when stirring of the aqueous solution during coacervation is slowed or stopped. Macrocapsules are then shaped into plaques for insertion into an eye.

In an alternative method of making the implants, a membrane coating may be formed around the layered solution to provide an encapsulated implant for controlled, prolonged release of the active agent. To form the coating, an appropriate aqueous solution, generally water, is slowly poured over the surface. In this manner, polymerization results in a membrane surrounding the drug or agent. The resulting membrane bound plaques can be cut to any size or geometry for incorporation into an attenuation device. To produce sheets of a particular dimension, the solution can be layered into preformed molds and the surface polymerized. Alternatively, the drug and polymer mixture may be extruded to provide, for example, a long rod or fiber. The fiber may then be cut to pieces of desired length for incorporation into the final device.

The dispersion or solution can alternatively be added to a rapidly stirred aqueous solution comprising water and a dispersion agent, which may be a protective colloid. To form macromolecules, dispersing agents such as poly(vinyl alcohol) (1 to 5%) or non-ionic detergents, such as Span detergents are employed.

Implants may also be formed by mixing the agent with molten polymer at the appropriate temperature, for example for molten polylactic polymer, between 60° to 90° C. The resulting mixture can be cut, molded, injection molded or extruded into any shape or size for incorporation into an attenuation device.

The implants may also be formed by pouring or layering the active agent dispersion or solution onto a surface such as a petri plate. By variation of surface area in relationship to the volume of polymer solution, the layer can be made to conform to any desired dimensions including surface area and width. For ease in handling of the implant, the polymer solution may be directly layered onto a release liner. Where desired, the release liner may comprise an adhesive layer on the side of the liner in contact with the polymer solution. After evaporation of the solvent, a second release liner may be employed to protect the exposed portion of the implant. Where a backing layer is to be employed, the polymer solution may be layered directly onto the backing layer material and the solvent evaporated or a release liner attached to the underlying structure. Where a membrane layer is desired, a solution of the membrane polymer may be layered over the polymer layer. Where desired, a release liner may then be placed on top of the polymer layer and/or the membrane layer. Where the implant is to comprise an adhesive layer, the adhesive layer may be applied to the release liner prior to placing the release liner on the polymer layer and/or membrane layer. When the release liner is later removed prior to insertion of the implant, the adhesive layer will substantially remain on the polymer layer and/or membrane layer.

Where desired, the implant may be formed by one of the methods described above, but in the absence of the active agent. The drug-free implant may then be loaded with drug by, for example, immersing the implant in a solution comprising the active agent for a time sufficient for absorption of the drug. Alternatively, where the implant incorporates a hollow fiber, for example, the active agent may be directly loaded into the fiber and the implant subsequently sealed. Where the activity of the drug will not be compromised, the drug-filled implant may then be dried or partially dried for storage until use. This method may find particular application where the activity of the drug of choice is sensitive to exposure to solvents, heat or other aspects of the conventional solvent-evaporation, molding, extrusion or other methods described above.

Where an implant comprising a refillable reservoir is desired, implant may be molded in two separate portions. At least one of these separate portions may be substantially concave. The two portions, which comprise the body of the implant, may then be sealed together with a biocompatible adhesive, such as a silicone adhesive, to form an implant having a substantially hollow center which may serve as a reservoir or depot for the active agent or drug. Alternatively, implants comprising a reservoir may be produced by conventional form-fill-seal techniques. Where an inlet is desired, the inlet may be positioned in the implant prior to sealing. The refillable implant may also be manufactured employing injection molding techniques. By employing injection molding, the shape and size of the implant, the desired volume of active agent to be held within the reservoir, the presence of an inlet for refilling the implant and the like may be varied by varying the mold which receives the polymer mixture. The refillable implant may be filled with the active agent or active agent suspension after the non-biodegradable outer layer is formed. Alternatively, the implants may be co-molded so that the outer non-biodegradable surface and the biodegradable-active agent center are formed substantially simultaneously by, for example, co-injection into a mold during injection molding.

In order to define the potential drug-release behavior of the implants in vivo, a weighed sample of the implants may be added to a measured volume of a solution containing four parts by weight of ethanol and six parts by weight of deionized water. The mixture is maintained at 37° C. and stirred slowly to maintain the implants in suspension. The appearance of the dissolved drug as a function of time may be followed spectrophotometrically until the absorbance becomes constant or until greater than 90% of the drug has been released. The drug concentration after 1 h in the medium is indicative of the amount of free unencapsulated drug in the dose, while the time required for 90% drug to be released is related to the expected duration of action of the dose in vivo. As a general rule, one day of drug release is approximately equal to 35 days of release in vivo. While release may not be uniform, normally the release will be free of larger fluctuations from some average value which allows for a relatively uniform release, usually following a brief initial phase of rapid release of the drug.

The implants may be administered into the eye in a variety of ways, including surgical means, injection, trocar, etc.

The implants may be anchored in the conjunctiva or sclera; episclerally or intrasclerally over an avascular region; substantially within the suprachoroidal space over an avascular region such as the pars plana or a surgically-induced avascular region; or in direct communication with the vitreal chamber or vitreous so as to avoid diffusion of the drug into the bloodstream. In this way, the device actively responds to high frequency pressure bursts in the eye using the attenuator portion of the device while also delivering active therapeutic agents to further mitigate underlying disease.

To eliminate pain and irritation of the eye, the shape of the attenuation device can change to conform to the eye wall in order to maximize the surface area of the attenuation device in contact with the eye wall so as to dissipate the pressure over as large a surface area of the eye wall as possible, and thereby prevent the focal points that cause trauma, pain, or irritation to the eye. In one embodiment, the attenuation device has a compressible wall on one side with a more rigid or form-shaping wall on the other side. The compressible wall will still enable the device to attenuate pressure fluctuations within the eye but the non-compressible wall will ensure good device placement and retention.

The embodiments have been described for use in the human anatomy. As understood by those skilled in the art, the present invention is not limited to human use; rather appropriately scaled versions of the inventions disclosed herein can be used to provide clinical benefits to other animals, including but not limited to mammalian household pets.

Certain embodiments provide significant advantages over prior art devices and methods. These advantages include but are not limited to: significant reductions in eye dysfunction related events; the ability to retrain a eye with other than normal compliance; no patient interaction required to operate or maintain the attenuation device; no infection conduit; minimal sensation generated by the attenuation device; low cost to manufacture; cost effective solution for patient when compared to existing treatments; and ease of installation and removal for clinician.

In one embodiment, there is provided a method of treating a patient with glaucoma, comprising the step of attenuating an increase in pressure within the eye by reversibly reducing the volume of the attenuator in response to the pressure.

In another embodiment, there is provided a method of improving the symptoms of glaucoma, comprising advancing a compressible device into the eye.

In another embodiment, there is provided a method of improving the symptoms of glaucoma, comprising positioning a device within the eye.

In another embodiment, there is provided a device for treating symptoms of glaucoma, comprising a compressible attenuator having an expanded volume within the range of from about 0.01 cc to about 0.5 cc if placed in the anterior chamber, and a valve for permitting filling of the attenuator through a filling device. Alternatively, if placed in the posterior chamber, the attenuator could have a volume ranging from 0.01 cc to 0.6 cc. Alternatively, if placed in the vitreous humor, the attenuator could have a volume ranging from 0.01 cc to 5 cc.

In another embodiment, there is provided a method of treating a patient, comprising the step of providing a compressible attenuator which is moveable from a first, introduction configuration to a second, implanted configuration.

In another embodiment, there is provided a device for treating symptoms of glaucoma, comprising a compressible attenuator having an expanded volume within the range of from about 0.01 cc to about 7 cc, and a valve having a first membrane and a second membrane with a flow passage there between for filling the attenuator.

In another embodiment, there is provided a device for reducing peak pressures in the eye.

In another embodiment, there is provided a device for increasing the compliance of the eye.

In another embodiment, there is provided a device for reducing local wall stresses.

In another embodiment, there is provided a device for reducing wall movement and stretching.

In another embodiment, there is provided a device for reducing pressure exerted on the optic nerve.

In another embodiment, there is provided a device for reducing pressure exerted on the retina.

In another embodiment, there is provided a device for reducing pressure exerted on the blood vessels within the eye.

In another embodiment, there is provided a device for reducing anterior chamber pressure changes.

In another embodiment, there is provided a device for reducing posterior chamber pressure changes,

In another embodiment, there is provided a device for reducing vitreal chamber pressure changes.

In another embodiment, there is provided a device for reducing posterior/anterior pressure mismatches.

In another embodiment, there is provided a method of placing the pressure attenuator within the eye.

In another embodiment, there is provided a method for placing the device within the anterior chamber comprising the steps of forming a sub 3 mm incision in the cornea and placing the device into the anterior chamber using a cannula, a syringe or a catheter. The device may be placed in a folded state and then unfolded in the anterior chamber, inserted in an uninflated state and then inflated in the anterior chamber, placed in unfolded or in an inflated state in the anterior chamber.

In another embodiment, an attenuator device may be placed within the anterior chamber and may be anchored or un-anchored within the anterior chamber. In addition, the attenuator might have haptics that anchor into the iris as described in Willis 7,008,449, Cumming 6,051,024, and Worst 5,192,319 by penetrating the iris or by means of a rivet, staple, clasp or other means. In another embodiment, the attenuator is placed in the anterior chamber and anchored into the trabecular meshwork by means of penetrating the meshwork with an anchor or by means of a rivet, staple, clasp, suture or other means. In another embodiment, the attenuator floats freely in the anterior chamber and is optically transparent.

In another embodiment, the attenuator may be attached/integral to any existing anterior chamber devices, such as a phakic intraocular lens or a shunt. In another embodiment, the attenuator is part of a drainage device which is implanted through an incision in the sclera, with the drainage tube sitting in the anterior chamber of the eye. The attenuator could be part of any portion of the device, but in a preferred embodiment is part of the portion anchored in the sclera. In another embodiment, the attenuator is part of the drainage tube that sits in the anterior chamber.

In another embodiment, a sub 3 mm incision is made in the cornea, the pupil is dilated and the attenuator is placed in the posterior chamber but anterior to the capsular bag (posterior to the iris).

In another embodiment, a sub 3 mm incision is made in the cornea, standard phacoemulsification of the native lens is performed and the attenuator is placed into the capsular bag in the posterior chamber.

In another embodiment, there is provided that the device may be anchored or un-anchored within the posterior chamber. In another embodiment, the device is placed in the posterior chamber, anterior to the capsular bag and posterior to the iris, and floats freely with no anchors. In another embodiment, the device is placed in the posterior chamber, and anchors to the posterior side of the iris by means of hooks, anchors, rivets, clasps, sutures, staples or other means.

In another embodiment, the attenuator is placed into the capsular bag in the posterior chamber and contains an intraocular lens

In another embodiment, the device is placed into the capsular bag in the posterior chamber and is part of the haptics of the intraocular lens

In another embodiment, the pupil is dilated and the device is inserted through a less than 3 mm incision in the cornea. The device unfolds and is placed posterior to the iris and anterior to the capsular bag which holds the crystalline lens. The attenuator is an Air/gas or mechanical device. The attenuator is anchored or un-anchored: the device sits posterior to the iris but anterior to the capsular bag. In another embodiment, the device floats freely. In another embodiment, the device is anchored to the posterior portion of the iris, by means of hooks that penetrate the iris, rivets, staples or clasps. In another embodiment, the device may contain optics that are designed to enhance visual acuity or that offer no visual effect. In another embodiment, the natural crystalline lens is removed from the capsular bag via phacoemulsification or other means, and the attenuator is implanted into the capsular bag in the posterior chamber. In this embodiment, the attenuator may be optically transparent, unable to travel into the optical path, or designed to enhance visual acuity. The attenuator may be attached/integral to any existing posterior chamber devices, such as a posterior chamber phakic intraocular lens or a non-phakic intraocular lens. In the case of a posterior chamber phakic IOL, a preferred embodiment has the attenuator portion of the device surrounding the optics. In another embodiment, the attenuator portion is integrated into the optics.

In another embodiment, the device is inserted into the vitreous by means of a trans pars plana approach between the iris and the retina.

In another embodiment, there is provided that the attenuator may be anchored or unanchored within the vitreous humor. The attenuator may anchor to the wall of the vitreous chamber by means of a rivet, staple, clasp, suture, or other means. The attenuator may float freely in the vitreous chamber. In another embodiment, the attenuator may be optically transparent. In another embodiment, the attenuator may not travel through the optical path. The attenuator may be inserted with a syringe, cannula, catheter or an introducer or inserter-type device, similar to that used for intraocular lenses.

The attenuator may be external and in communication with anterior chamber

The attenuator may by external and in communication with posterior chamber

The attenuator may be in direct communication with both chambers

The attenuator may be external and in communication with the vitreous chamber.

The attenuator may contain a pressure transducer to continuously record intraocular pressure.

The attenuator may be linked or communicate with a pressure transducer that continuously records intraocular pressure.

The attenuator may be coated to prevent inflammation, encapsulation, or the growth of biofilm.

The attenuator may be coated with a drug to prevent inflammation, encapsulation or the growth of a biofilm.

In another embodiment, there are provided methods and devices for the restoration of dynamic compliance of the eye by retraining the eye tissue by introducing pressure waves at a prescribed place and with prescribed characteristics.

In another embodiment, there are provided methods and devices for the programmatic delivery of clinical therapeutics in association with defined pressure events. Such devices could be added to other intraocular devices, such as shunts, intraocular lens, or phakic intraocular lens.

In another embodiment, there is provided an atraumatic method of measuring intraocular pressure without the need for any external connection by placing a pressure transducer and telemetry device within the attenuation device. This secures the transducer within the eye and prevents the need to attach the transducer to the eye wall.

In another embodiment, the attenuation device is used in the field of opthalmology to support cranio-facial tissue during healing after a traumatic event or intraoptically as therapy for acute angle closure glaucoma.

In another embodiment, there are provided air cell-like attenuation devices that are placed in the eye and/or other organs of the body and filled with or comprise one or more compressible substances to provide pressure compensation. Additionally, active, programmable pressure compensators or generators are envisioned to monitor pressure events, respond in a predetermined fashion, and record or transmit that information outside the body. Additionally, a reliable, maintenance-free therapeutic delivery system is described to programmatically release or distribute an agent into an organ of the body using an erodable or deformable support matrix or material of construction, and/or a programmable or responsive valving system.

In another embodiment, there is provided an attenuation device that may assume multiple shapes during the course of its use. For example, the attenuation device may be completely deflated for introduction and inflated to varying degrees after introduction. The attenuation device may be adjusted through the inflation/deflation of secondary or multiple containment cells for such purposes as ballasting or the addition of a diagnostic, therapeutic or signaling substance. This may occur through multiple uses of a single, or single uses of a multi lumen, multi ported structure or combinations thereof.

In another embodiment, an opthalmoscope may be used to launch and retrieve the device (i.e. attenuation device, accumulator, etc.).

In another embodiment, the distal tip of the delivery system may be straight, pro curved, malleable, or steerable (e.g., by pull wires) in order to facilitate delivery and/or release of the device.

In another embodiment, the separation of the attenuation device from the fill tube may be accomplished using the wall of the eye-as a mechanically resistant body.

In another embodiment, the delivery system may consist of a single tubular element, a series of concentric tubular elements, a series of non-concentric tubular elements, an extruded element, a spirally wound guidewire element, or any combination of the aforementioned elements arranged in a manner to provide the desired functions.

In another embodiment, irritation concerns are addressed through the use of coatings or fillers to physically or chemically modify the attenuation device in whole or part in order to modulate characteristics such as lubricity and the ability to inhibit the deposition of materials present in the eye. For example, substances such as sulfated polysaccharides may be used before, during, or after introduction to the patient. In addition, the use of a plurality of construction materials with unique surface properties may also be used for this purpose.

In another embodiment, the attenuation device includes a portal that spans the distance from the internal aspect to the external aspect that allows for the location of an erodable substance that would allow for the deflation or deconstruction of the attenuation device after exposure to intraocular conditions for a prescribed period of time. This approach may also be used for the programmed bolus release of single or multiple therapeutic, diagnostic or signaling substances from single or multiple chambers within the attenuation device.

In another embodiment, the attenuation device is equipped with a valve/port that is programmable, self-regulating or responsive to stimuli, which may or may not be physiological. Telemetry, physical connection or remote signaling may be used to elicit a desired response.

In another embodiment, the attenuation device accepts, captures, and/or translates physical forces within the eye to energize a site within the attenuation device for the positive displacement of substances outside the boundary of the attenuation device in either continuous or bolus presentation.

The embodiments described herein are not limited to intraocular devices, but also include devices and methods for controlling pressure transients in other organs of the body.

Having thus described certain embodiments of the present invention, various alterations, modifications and improvements will be apparent to those of ordinary skill in the art. Such alterations, variations and improvements are intended to be within the spirit and scope of the present invention. Accordingly, the foregoing description is by way of example and is not intended to be limiting. In addition, any dimensions that appear in the foregoing description and/or the figures are intended to be exemplary and should not be construed to be limiting on the scope of the present invention described herein. It should further be understood, however, that the invention is not to be limited to the particular forms or methods disclosed, but to the contrary, the invention is to cover all modifications, equivalents and alternatives falling within the spirit and scope of the appended claims.

1. An attenuation device, comprising:
a flexible housing comprising an outer wall and defining a chamber therein, wherein the chamber having an expanded volume within a range from about 0.01 cc to about 7 cc and being positionable within a patient's eye; and
at least one high vapor pressure media having a vapor pressure approximately equal to an intraocular pressure of the patient's eye and a permeability of less than 1 ml/day at body temperature through the outer wall.

2. The attenuation device of claim 1, wherein the high vapor pressure media comprises heptafluoropropane.

3. The attenuation device of claim 1, wherein the high vapor pressure media comprises perfluorooctylbromide.

4. The attenuation device of claim 1, wherein the high vapor pressure media comprises perfluorohexane.

5. The attenuation device of claim 1, wherein the high vapor pressure media comprises perfluorodecalin.

6. The attenuation device of claim 1, wherein the high vapor pressure media comprises tetrafluoroethane.

7. The attenuation device of claim 1, wherein the high vapor pressure media comprises sulfur hexafluoride.

8. The attenuation device of claim 1, wherein the high vapor pressure media comprises hexafluoroethane.

9. The attenuation device of claim 1, wherein the high vapor pressure media comprises perfluoropropane.

10. The attenuation device of claim 1, wherein the high vapor pressure media comprises perfluorobutane.

11. The attenuation device of claim 1, wherein the high vapor pressure media comprises perfluoropentane.

12. The attenuation device of claim 1, wherein the high vapor pressure media comprises perfluoroheptane.

13. The attenuation device of claim 1, wherein the high vapor pressure media comprises perfluorooctane.

14. The attenuation device of claim 1, wherein the high vapor pressure media comprises octafluoropropane.

15. The attenuation device of claim 1, wherein the high vapor pressure media comprises decafluoro-n-butane.

16. The attenuation device of claim 1, wherein the high vapor pressure media comprises perfluoroperhydrophenanthrene.

17. The attenuation device of claim 1, wherein the high vapor pressure media is a liquid at body temperature.

18. The attenuation device of claim 17, wherein the density of the high vapor pressure media is greater than that of the ocular fluid wherein the device is placed.

19. The attenuation device of claim 18, wherein the degree to which the high vapor pressure media counteracts the buoyancy of any gas in the attenuation device is determined by the amount of the high vapor pressure media in the attenuation device.

20. The attenuation device of claim 19, wherein the reduced buoyancy of the attenuation device results in reduced pressure on the structures of the eye.

21. The attenuation device of claim 1, wherein the high vapor pressure media has a solubility of less than about 0.1 ml per ml of ocular fluid at body temperature and pressure.

22. A method of treating a patient, comprising the steps of:
introducing a compressible attenuation device which is moveable from a first, introduction configuration to a second, implanted configuration, into the eye while in the first configuration;
transforming the attenuation device within the eye to the second configuration, wherein the second configuration having a volume within a range from about 0.01 cc to about 7 cc; and
attenuating a pressure change within the eye by reversibly changing the volume of the attenuation device in response to the pressure change.

23. The method of claim 22, wherein the step of transforming the attenuation device to the second configuration comprises introducing within the attenuation device at least one high vapor pressure media.

24. The method of claim 22, wherein the step of introducing the attenuation device step comprises transclerally or transcorneally introducing the attenuation device into the eye.

25. The method of claim 24, wherein the attenuation device is attached to a structure in the eye.

26. The method of claim 24, wherein the attenuation device is free floating within the eye.

27. The method of claim 22, wherein the step of introducing the attenuation device step comprises transclerally introducing the attenuation device into the vitreous chamber of the eye.

28. The method of claim 22, wherein the step of introducing the attenuation device step comprises transcorneally introducing the attenuation device into the anterior or posterior chambers of the eye.

29. The method of claim 23, wherein the high vapor pressure media has a vapor pressure approximately equal to the intraocular pressure of the eye and a permeability of less than 1 ml/day at body temperature through the outer wall of the attenuation device.

30. The method of claim 22, wherein the high vapor pressure media comprises heptafluoropropane.

31. The method of claim 22, wherein the high vapor pressure media comprises perfluorooctylbromide.

32. The method of claim 22, wherein the high vapor pressure media comprises perfluorohexane.

33. The method of claim 22, wherein the high vapor pressure media comprises perfluorodecalin.

34. The method of claim 22, wherein the high vapor pressure media comprises tetrafluoroethane.

35. The method of claim 22, wherein the high vapor pressure media comprises sulfur hexafluoride.

36. The method of claim 22, wherein the high vapor pressure media comprises hexafluoroethane.

37. The method of claim 22, wherein the high vapor pressure media comprises perfluoropropane.

38. The method of claim 22, wherein the high vapor pressure media comprises perfluorobutane.

39. The method of claim 22, wherein the high vapor pressure media comprises perfluoropentane.

40. The method of claim 22, wherein the high vapor pressure media comprises perfluoroheptane.

41. The method of claim 22, wherein the high vapor pressure media comprises perfluorooctane.

42. The method of claim 22, wherein the high vapor pressure media comprises octafluoropropane.

43. The method of claim 22, wherein the high vapor pressure media comprises decafluoro-n-butane.

44. The method of claim 22, wherein the high vapor pressure media comprises perfluoroperhydrophenanthrene.

45. The method of claim 22, wherein the high vapor pressure media is a liquid at body temperature.

46. The method of claim 45, wherein the density of the high vapor pressure media is greater than that of the ocular fluid.

47. The method of claim 46, wherein the degree to which the high vapor pressure media counteracts the buoyancy of any gas in the attenuation device is determined by the amount of the high vapor pressure media in the attenuation device.

48. The method of claim 47, wherein the reduced buoyancy of the attenuation device results in reduced pressure on the structures of the eye.

49. The method of claim 23, wherein the high vapor pressure media has a solubility of less than about 0.1 ml per ml of ocular fluid at body temperature and pressure.

50. The method of claim 22, wherein the transforming step comprises at least partially inflating the attenuation device.

51. The method of claim 22, wherein the transforming step comprises permitting the attenuation device to transform under its own bias.

52. The method of claim 22, wherein the attenuating step comprises reducing the volume of the attenuation device by at least about 5%.

53. The method of claim 22, wherein the attenuating step comprises reducing the volume of the attenuation device by at least about 10%.

54. The method of claim 22, wherein the attenuating step comprises reducing the volume of the attenuation device by at least about 25%.

55. The method of claim 22, wherein the attenuation is accomplished by a reduction in volume of the attenuation device.

56. The method of claim 53, wherein the reduction in volume is responsive to the increase in pressure.

57. The method of claim 22, wherein the attenuation device comprises a compressible wall.

58. The method of claim 22, wherein the step of attenuating a pressure change includes attenuating an intraocular pressure spike that would have been at least about 21 mm Hg without the presence of the attenuation device to a pressure of no more than about 20 mm Hg.

59. The method of claim 22, wherein the step of attenuating a pressure change includes attenuating an intraocular pressure spike that would have been at least about 30 mm Hg without the presence of the attenuation device to a pressure of no more than about 25 mm Hg.

60. The method of claim 22, wherein the step of attenuating a pressure change includes attenuating an intraocular pressure spike that would have been at least about 40 mm Hg without the presence of the attenuation device to a pressure of no more than about 30 mm Hg.

61. A method of treating a patient with retinal detachment, comprising the steps of advancing a compressible device into the patient's eye and attenuating a pressure change within the eye by reversibly changing the volume of the compressible device in response to the pressure change.

62. A method of protecting a patient's ocular tissues from temporary or transient pressure spikes during ocular procedures, comprising the steps of
introducing a compressible device into a eye prior to conducting an ocular procedure,
performing the ocular procedure, and
attenuating a pressure change within the eye during the ocular procedure by reversibly changing the volume of the compressible device in response to the pressure change.

63. A method of improving the symptoms of glaucoma in a patient, comprising the steps of
positioning a compressible device within a chamber of the patient's eye, and
inhibiting a decrease in compliance of the eye.

64. The method of claim 63, wherein the positioning step comprises trans-corneally or trans-sclerally introducing the compressible device into the eye.

65. A compressible attenuator device for treating symptoms of glaucoma, comprising a compressible chamber having an expanded volume within the range of from about 0.01 cc to about 7 cc, and a valve for permitting filling of the chamber through a filling device; wherein the valve has a first pair of complementary surfaces for resisting deflation of the chamber, and a second pair of complementary surfaces for resisting additional filling of the chamber when the chamber is exposed to an external pressure which is greater than an internal pressure within the attenuator.

66. The device of claim 65, wherein the chamber is compressible to no more than about 80% of its expanded volume under a pressure of about 50 mm Hg.

67. The device of claim 65, wherein the first pair of complementary surfaces are opposing surfaces on a flapper valve.

68. The device of claim 67, wherein the flapper valve is oriented such that an increase in the internal pressure increases the closing pressure on the flapper valve.

69. The device of claim 68, wherein the flapper valve extends into the chamber.

70. The device of claim 65, wherein closing pressure on the second pair of complementary surfaces is increased in response to an increase in pressure in the external environment surrounding the attenuator device.

71. A device for attenuating pressure increases in a patient's eye comprising a body having a compressible region extending from an optically clear region, wherein the body is positionable within a chamber of the eye with the optically clear region crossing the optical axis of the eye.

72. The device of claim 71 wherein the compressible region has an expandable volume in a range from about 0.01 cc to about 7.0 cc.

73. The device of claim 71 wherein the optically clear region comprises a baffle coupled to the compressible region.

74. The device of claim 73 wherein the baffle comprises a sheet of material with a plurality of holes formed therein.

75. The device of claim 71 wherein the optically clear region comprises a lens.

76. The device of claim 75 wherein the compressible region forms haptics coupled to the lens.

77. The device of claim 75 wherein the lens has the same index of refraction as the ocular fluid.

78. The device of claim 75 wherein the lens has an index of refraction greater than the ocular fluid.

79. The device of claim 75 wherein the lens has an index of refraction less than the ocular fluid.

80. The device of claim 75 wherein the lens has a single focal length

81. The device of claim 80 wherein an optic the lens moves with muscular movement for pseudo-accommodation.

82. The device of claim 75 wherein the lens has more than one focal length within the optic

83. The device of claim 82 wherein an optic of the lens moves with muscular movement for pseudo-accommodation.

84. The device of claim 76 wherein the lens has a single focal length

85. The device of claim 84 wherein an optic of the lens moves with muscular movement for pseudo-accommodation.

86. The device of claim 76 wherein the lens has more than one focal length within the optic.

87. The device of claim 86 wherein an optic of the lens moves with muscular movement for pseudo-accommodation.

88. The device of claim 71 further comprises an anchor extending from the compressible region and coupling the compressible region to a structure of the eye.

89. The device of claim 88 wherein the anchor comprises a valve to fill and deflate the compressible region.

90. The device of claim 89 wherein the valve comprises a cap.

91. The device of claim 71 further comprising a component biodegradable in the presence of ocular fluid.

92. A method of improving the compliance of the structures of a patient's eye, comprising the steps of
inserting a pressure attenuation device within a chamber of the patient's eye, and
unloading the mechanical stress and strain in the structures of the eye.

93. The method of claim 92, wherein the step of unloading includes reducing wall stress in the walls of the eye.

94. The method of claim 92, wherein the step of unloading includes increasing outflow of ocular fluid.

95. The method of claim 92, wherein the step of unloading includes at least partially restoring accomodation.

96. The method of claim 92, wherein the step of unloading includes moderating or reducing ocular hypertension.

97. The method of claim 92, wherein the step of unloading includes attenuating pressures that cause retinal tears.

98. The method of claim 92, wherein the step of unloading includes moderating or reversing retinal ischemia.

99. The method of claim 92, wherein the step of unloading includes moderating or reversing retinal vein or artery occlusion.

100. The method of claim 92, wherein the step of unloading includes moderating or reversing macular edema.

101. The method of claim 92, wherein the step of unloading includes moderating or reversing diabetic retinopathy.

102. The method of claim 92, wherein the step of unloading includes moderating or reversing neovascularization.

103. The method of claim 92, wherein the step of unloading includes moderating or reversing macular degeneration.

104. The method of claim 92, wherein the step of unloading includes moderating or reversing myopia.

105. The method of claim 92, wherein the step of unloading includes moderating or reversing hyperopia.

106. The method of claim 92, wherein the step of unloading includes moderating or reversing presbyopia.

107. The method of claim 92, wherein the inserting step comprises trans-corneally or trans-sclerally introducing the attenuation device into the eye.

108. The method of claim 92 wherein the inserting step includes inserting a drainage device in addition to the attenuation device.
